(19) United States
(12) Patent Application Publication
(10) Pub. No.: US 2004/0234450 A1
(43) Pub. Date: Nov. 25, 2004

(54) Compositions, methods, apparatuses, and systems for singlet oxygen delivery

(76) Inventor: Randolph M. Howes, Kentwood, LA (US)

Correspondence Address: CALFEE HALTER & GRISWOLD, LLP, 800 SUPERIOR AVENUE, SUITE 1400, CLEVELAND, OH, 44114, US

(21) Appl. No.: 10/874,438

(22) Filed: Jun. 23, 2004

Related U.S. Application Data
(62) Division of application No. 10/331,773, filed on Dec. 31, 2002.
Application No. 10/331,773 is a continuation-in-part of application No. 10/050,121, filed on Jan. 18, 2002.
Application No. 10/050,121 is a continuation-in-part of application No. 10/023,754, filed on Dec. 21, 2001.
(60) Provisional application No. 60/262,635, filed on Jan. 22, 2001.

(51) Int. Cl. A61K 51/00; A61M 36/14; A61K 33/40

(52) U.S. Cl. 424/1.11; 424/616

(57) ABSTRACT

Methods of treating tumors, lesions, and cancers comprising delivering to the affected site a combination of peroxide and hypochlorite anion. Hydrogen peroxide and sodium hypochlorite are possible sources of peroxide and hypochlorite anion, respectively. The reactants may be injected simultaneously or sequentially, and combine at the site to produce singlet oxygen. Singlet oxygen may be delivered to the treatment site or generated at the treatment site. Isotopes are also synergistically used in conjunction with singlet oxygen. The isotopes may be radioactive isotopes, non-radioactive isotopes, or both.

### RELATED APPLICATIONS

This application is a continuation-in-part of a U.S. patent application Ser. No. 10/050,121, filed Jan. 18, 2002, which is a continuation-in-part of a U.S. patent application Ser. No. 10/023,754, filed Dec. 21, 2001, and further claims priority under 35 U.S.C. § 119(e) to U.S. provisional application No. 60/262,635, filed Jan. 22, 2001, the entire disclosure of each of which is incorporated herein by reference.

### FIELD OF THE INVENTION

The present invention relates to methods, apparatuses, and systems for singlet oxygen delivery. In particular, the present invention relates to methods of providing singlet oxygen delivery comprising administering a source of peroxide and a source of hypochlorite, as well as systems and apparatuses for use in the method. The source of peroxide may be hydrogen peroxide, and the source of hypochlorite may be sodium hypochlorite. In addition, the present invention relates to singlet oxygen producing systems, which may include or be used with isotopes. The isotopes may be radioactive isotopes, non-radioactive isotopes, or both.

### BACKGROUND

Control and destruction of unwanted living organisms is a critical part of healthcare throughout the world. Pathogens, such as bacteria, viruses, fungi, unicellular organisms, and multicellular organisms, which normally live outside a person, can become destructive or life-threatening if allowed to take hold and reproduce in or on a person. Enormous resources, in the United States and abroad, are allocated to the control and destruction of such pathogens.

Sterilants and disinfectants may be considered a first line of defense, killing pathogens in an environment outside a living body. These products are intended to kill pathogens before they ever have an opportunity to contact a person and generate an infection. Household and industrial cleaning products are well known examples that frequently include an antimicrobial agent to reduce the population of pathogens. Traditionally, such products have been important for use in areas of food preparation or consumption, such as kitchens or restaurants, and in areas where pathogens are more likely to be found, such as bathrooms or locker rooms. Sterilants and disinfectants have been especially important in areas in which control of pathogens is critical, such as in medical treatment facilities, including veterinary and human facilities, hospitals, and in particular, operating rooms in such facilities. More recently, and in particular following the recent events in the United States, sterilants and disinfectants have been used to decontaminate areas that have been exposed to biological weapons such as anthrax.

Generally, sterilants and disinfectants are toxic or corrosive and thus can only be applied to inert surfaces, not directly to people or animals. That is, their toxicity generally precludes their application directly onto people or animals, where the toxicity would be too great. However, other compositions that may be applied directly to people and animals do exist and are commonly used. These compositions are often referred to as antiseptics.

Antiseptic agents are generally used in controlling or reducing the population of pathogens that have already contacted a living being, or in areas where prophylaxis is important. For example, topical antiseptics are applied to skin abrasions and wounds to prevent infection. Antiseptics are also formulated in washes, such as in shampoos, soaps, or detergents, which may be used to topically reduce and control pathogen population. Antiseptic formulations, however, are generally too toxic to be taken internally by humans or animals.

Antibiotic compositions may be administered to humans and animals. Such compositions generally exhibit a high degree of pathogen toxicity, yet are formulated to minimize human and animal toxicity. These compositions can be used when pathogens breach a body's protective defenses. Diseases produced by pathogens are well known, as are the antibiotics often used in their treatment. Antibiotics, such as dactinomycin, daunorubicin, doxorubicin, and the bleomycins, have also been used in treating diseases such as cancer by targeting abnormally proliferating cells.

Cancer remains one of the leading causes of death in the United States and the world. Treatment of cancer focuses on killing cancerous cells, yet avoiding the significant side effect of death to surrounding healthy cells. While improvements have been made in the area of cancer treatment, surgery, radiotherapy, and chemotherapy, each is still associated with significant side effects and limitations. And the side effects, such as toxicity and immunosuppression, often further contribute to patient illness and hamper the patient's ability to recover. Thus, efforts at developing new treatments aim to maximize effectiveness while minimizing side effects and reduce the overall worldwide cancer death rate.

A newer method that has had some success in maximizing effectiveness and minimizing side effects is photodynamic therapy (See Dougherty, T. J., Photodynamic Therapy—New Approaches, Semin. Surg. Oncol. 5(1): 6-16 (1989); Liberman J. Light, Medicine of the Future, Santa Fe: Bear & Co. (1991); Hopper, C., Photodynamic Therapy: A Clinical Reality in the Treatment of Cancer, Lancet Oncol. 1: 212-219 (2000)). This treatment generally involves infusing a photoactive compound into a patient and allowing the compound to collect in a tumor that is to be targeted. The photoactive compound in the tumor is irradiated with light energy (photons), thereby generating the killing compound, which is a short-lived oxygen specie called electronically excited singlet oxygen. The singlet oxygen is believed to produce toxic effects on the cells of the tumor through oxidation and/or free radical reactions. Photodynamic therapy has been effective in treating multiple types of cancer, including cancers of different tissues and organs, including benign and malignant tumors (See generally Oseroff, Photodynamic Therapy, Clinical Photomedicine, 387-402 (Marcel Dekker, Inc.) (1993)).

A similar technique has been used in the treatment of atherosclerosis, which is a type of arteriosclerosis (Grant, W. E. et al., The Effect of Photodynamic Therapy on the Mechanical Integrity of Normal Rabbit Carotid Arteries, Laryngoscope 105: 867-871 (1995)). The word “atherosclerosis” comes from the Greek words athero, meaning gruel or paste, and sclerosis, meaning hardness. The disease results from deposits of fatty substances, including cholesterol and cholesterol esters, as well as cellular waste products, calcium, and other substances on the inner lining of an artery. This build-up is called a plaque, and such plaques may grow large enough to significantly reduce blood flow through an artery and produce major ischemic problems, including stroke and/or death. The plaques can also become fragile and weaken vascular walls or produce microemboli.

Past attempts to prevent or treat damage caused by atherosclerosis included, for example, coronary artery bypass surgery, mechanical or laser plaque removal, balloon angioplasty, and placement of scaffolding stents. More recently, photodynamic therapy has been suggested as an alternative therapy. (See, for example, the news release dated Sep. 4, 2001, by Pharmacyclics, Inc. reported to the 23^{rd} Congress of the European Society of Cardiology, noting that Phase I clinical trials of photoangioplasty with Antrin (motexafin lutetium) was feasible and well tolerated.)

Photoactive compounds are useful because of their ability to produce singlet oxygen by absorbing light energy and becoming unstable. In their unstable form, photoactive compounds interact with oxygen to excite it from its stable triplet electron state to its excited singlet state, i.e., to singlet oxygen (¹O₂*). The singlet oxygen then produces the desired effect on the target area, be it cancer cells, atherosclerotic plaque tissue, and/or inflammation.

The efficient production of singlet oxygen using photodynamic therapy, thus, requires the presence of molecular oxygen at the target site. While this is not problematic at the beginning of the photodynamic reaction, it becomes problematic as the reaction progresses and oxygen is consumed and blood vessels to the area thrombose. As the reaction depletes oxygen in the target area, the reaction rate is reduced. And as the reaction entirely depletes oxygen from the target tissue, the reaction entirely ceases to produce the desired end product, singlet oxygen. Once in this anoxic state, the tissue is not further affected by the photodynamic therapy, other than by the undesirable side effects of residual photosensitizer compounds.

Attempts to overcome this limitation have included cycling the irradiation with light, i.e., periods of light exposure followed by periods of dark, thereby allowing ground state molecular oxygen to diffuse into the target tissue following a reaction period and allowing the reaction to reoccur. Oxygen loading, another technique, attempts to increase oxygen concentration in the patient's blood through use of hyperbaric conditions. Thus, oxygen is enriched at the tumor site, and the photodynamic effect is initially enhanced. However, as both of these methods merely provide temporary solutions, neither truly solves the drawbacks of photodynamic therapy.

Another difficulty in photodynamic therapy arises from the fact that a photoactive agent is injected into the body and then left to circulate. While it is desirable that the compound collect in the tumor tissue, this effect varies between individual patients. It is thus difficult to determine the appropriate light energy to be applied when the amount of photoactive agent varies between patients, tissues, and/or cell types. Also, because the agent is left to circulate in the body, significant photosensitivity occurs. Thus, untargeted portions of the body are unintentionally treated upon exposure to sunlight.

Photodynamic therapy is also limited by the need for a highly focused, light-generating system, which is usually provided by a laser. Laser penetration of tissue is limited to approximately 3 centimeters, making large or deep tissue tumors more difficult to treat with photodynamic therapy. Also, although laser use has become very common in medical applications, some technical expertise in laser operation is still necessary. Moreover, medical-quality lasers, even small ones, can be expensive. There is, therefore, a need in the art for a method of delivering singlet oxygen to a tumor target without the drawbacks associated with photodynamic therapy.

As a molecular specie, the existence of singlet oxygen has been recognized for years. In 1939, Kautsky (Trans. Faraday Soc. 35:216) proposed that an excited form of oxygen might be responsible for photooxidation reactions. Chemical studies that supported Kautsky's hypothesis were performed in the 1960s. Using a peroxide-hypochlorite anion system, Foote and Wexler (J. Amer. Chem. Soc. 86:3879 (1964)) demonstrated that products generated, including singlet oxygen, were identical to those obtained through dye-sensitized photooxidation. This reaction of hydrogen peroxide and hypochlorite to produce singlet oxygen was important in many of the early studies of singlet oxygen.

The reaction causes the decomposition of one molecule of hydrogen peroxide into one molecule of singlet oxygen and water. The reaction is shown below:

H₂O₂+ClO⁻—¹O₂*+Cl⁻+H₂O

Singlet oxygen is not foreign to the human body. Early work in the field by Howes and Steele (Res. Commun. Chem. Pathol. Pharmacol. 2:619-626 (1971); Res. Commun. Chem. Pathol. Pharmacol. 3:349-357 (1972)) suggested a possible involvement of singlet oxygen in liver microsomal hydroxylation reactions. Today, singlet oxygen is recognized as the principal bacterial oxidizing agent employed by the human neutrophil (macrophage) and monocyte phagosome. Although not entirely understood, it is believed that myeloperoxidase, hydrogen peroxide, and chloride combine to produce powerful oxidizing compounds, including singlet oxygen, in the phagosome. It has been proposed that the myeloperoxidase reacts with the hydrogen peroxide to form the singlet oxygen and hypochlorous acid.

The present invention takes advantage of the reaction between peroxide and hypochlorite to produce singlet oxygen. The present invention solves the aforementioned problems in photodynamic therapy, and also finds use in treating, for example, tumors, atherosclerotic plaques, inflammation sites, or mutated cells. And because its components are naturally occurring and safe, yet capable of a potent oxidizing potential, the present invention also finds use as a sterilant, disinfectant, antiseptic, and antibiotic.

### SUMMARY OF THE INVENTION

Features and Advantages of the Invention

This invention is advantageous in providing compositions, methods, apparatuses, and systems, for producing singlet oxygen.

It is advantageous that the singlet oxygen may be produced using chemical entities that are physiologically produced and physiologically present, without the need to resort to complex synthetic compounds, many of which have toxic or harmful side effects. The invention is advantageous in that the compositions and products of the singlet oxygen producing reaction are easily metabolized by body's natural metabolic mechanisms.

This invention is also advantageous in providing systems and methods for producing singlet oxygen including or in conjunction with isotopes, such as radioactive isotopes, non-radioactive isotopes, or both.

It is also advantageous that isotopes included in or in conjunction with singlet oxygen generating systems may be used for medical diagnosis, metabolic research, disease treatment, sterilization of food and medical products, tissue grafts, nutrition research, and industrial processes.

Moreover, singlet oxygen, including or in conjunction with, isotopes, may show synergistic effects in treating diseases, such as cancer, tumor, arteriosclerosis, or inflammation.

Furthermore, isotopes included in, or in conjunction with, singlet oxygen generating systems, may be used as tracers in chemical, biochemical, biological, industrial, and medical research.

It is also advantageous that isotopes used in accordance with the present invention may be linked to materials that can attach themselves to various types of target cells, such as cancer cells, thereby allowing the isotopes to be delivered directly to the locations of the target cells.

It is also advantageous that isotopes, in conjunction with singlet oxygen generating systems, may prolong the lifetime of singlet oxygen, thereby increasing its desired chemical reactivity and enhancing the effect of singlet oxygen generating systems.

The isotopes used in accordance with the present invention may show low toxicity to living organisms.

This invention may also be used as a system to dissolve abnormal growths and deposits, such as atherosclerotic or arteriosclerotic plaques, and may also have an additional desirable feature of treating associated hypertensive states and inflammatory sites.

This invention may be used as a disinfectant, decontaminating agent, containment agent, sterilant, antiseptic, antibiotic, and anti-inflammatory agent, and may be used on inert surfaces, as well as topically or internally for living organisms, including humans.

The invention may be used in decontaminating areas exposed to chemical or biological agents.

When used inside a living organism, this invention may be used to target the therapy at the desired site, without exposing the entire patient or the surrounding tissue to collateral damage, and the reactants and products decompose into well-tolerated physiological compounds.

This invention is also advantageous in providing singlet oxygen therapy to a site in need of therapy, using only simple surgical techniques, and without the need for expensive electronic equipment.

Additionally, the chemical constituents can be accurately regulated by concentration, rate of infusion, or infiltration and by precise depth of penetration.

It is also advantageous in that it does not have a limited depth of penetration and can be accurately administered at any desirable depth.

This invention is also advantageous in that it is not limited to the restrictions of penetration for photodynamic therapy.

In addition, the chemical yield of singlet oxygen may be accurately calculated.

It is also advantageous that this invention may be repeatedly administered without undue effects and in conjunction with standard cancer treatments, such as surgery, irradiation and/or chemotherapy.

### SUMMARY OF THE INVENTION

The present invention is directed to methods of treating a target site in or on a mammal, comprising administering a source of singlet oxygen, which may comprise administering at least one source of peroxide and at least one source of hypochlorite anion to the target site to be treated and allowing the peroxide and hypochlorite to react to produce singlet oxygen. In some embodiments, the source of peroxide comprises at least one of hydrogen peroxide, alkyl hydroperoxides, or metal peroxides.

In this invention, the source of hypochlorite anion may comprise at least one of metal hypochlorites or hypochlorous acid. Metal hypochlorites may be chosen from calcium hypochlorite, sodium hypochlorite, lithium hypochlorite, and potassium hypochlorite. The hypochlorite anion source may comprise chlorine dioxide.

In the present methods, the source of peroxide and source of hypochlorite anion may be administered sequentially. The source of peroxide and source of hypochlorite anion may be administered through at least one conventional syringe and needle. In the present invention, the source of peroxide and source of hypochlorite anion may also be administered simultaneously. In some embodiments, the source of peroxide and source of hypochlorite may be delivered through at least one dual lumen catheter.

The methods of the invention may be used where the target site is a tumor or an atherosclerotic plaque. The administration may be performed such that the source of peroxide and/or the source of hypochlorite anion is delivered upstream of blood flow to the target site and the blood flow carries at least one of the source of peroxide and the source of hypochlorite anion to the target site.

The invention is also directed to singlet oxygen produced by processes comprising a) introducing into a mammal at least one composition comprising at least one source of peroxide; and b) introducing into a mammal at least one composition comprising at least one source of hypochlorite anion.

This invention is also directed to systems for treating a target site in a mammal, comprising a) at least one source of peroxide; b) at least one source of hypochlorite anion; and c) at least one catheter having at least one lumen. The system may further comprise at least one syringe and at least one conduit. This system may be used, for example, where the target site is a tumor, an atherosclerotic plaque, a site of pathogenic infestation, amyloid deposits, or inflammation sites.

This invention is also directed to apparatuses for singlet oxygen delivery comprising a) a first reservoir for containing at least one peroxide source; b) a second reservoir for containing at least one hypochlorite anion source; c) a first conduit connecting the first reservoir to a delivery port; and d) a second conduit connecting the second reservoir to the delivery port. The apparatus may further comprise a mechanism to simultaneously deliver the peroxide source and the hypochlorite anion source, and/or a mechanism to control the flow of the peroxide source and the hypochlorite anion source from the first and second reservoirs through the first and second conduits to the delivery point. In apparatuses of this invention, the delivery port may be a catheter, or may be a spray nozzle, or may be any other delivery system.

The invention is further directed to apparatuses for singlet oxygen delivery comprising a) a first reservoir for containing a composition comprising at least one peroxide source; b) a second reservoir for containing a composition comprising at least one hypochlorite anion source; c) a first conduit connecting the first reservoir to a first delivery port; and d) a second conduit connecting the second reservoir to a second delivery port; wherein the first and second delivery ports are oriented to direct output to a target point. In some embodiments, the at least one peroxide source and the at least one hypochlorite anion source are solutions. As nonlimiting examples, the output may be a stream, or may be a mist. In some embodiments, the at least one of the compositions comprising at least one peroxide source or at least one hypochlorite anion source further comprises at least one surfactant.

This invention is also directed to methods for treating tumor cells or cancer cells as a result of seeding an operative site comprising administering as an irrigation or irrigating solution at least one source of peroxide and at least one source of hypochlorite anion. And the present invention is also directed to methods for killing pathogens in or on a mammal comprising administering an aqueous solution comprising at least one source of peroxide and an aqueous solution comprising at least one source of hypochlorite anion. In some methods of this invention, at least one of the aqueous solutions comprising at least one peroxide source and at least one source of hypochlorite anion further comprises at least one pharmaceutically acceptable excipient.

The invention is also directed to a singlet oxygen producing composition comprising a) at least one source of peroxide; b) at least one source of hypochlorite anion; and c) at least one of a surfactant, detergent, scent, colorant, viscosity-modifying agent, solvent, chelator, and pH-modifying agent. Methods of the invention also include disinfecting or decontaminating an inert area, comprising a) delivering at least one source of peroxide; b) delivering at least one source of hypochlorite anion; and c) delivering at least one of a surfactant, detergent, scent, colorant, viscosity-modifying agent, solvent, chelator, and pH-modifying agent. In methods of this invention, any of a), b), or c) may be performed separately, or simultaneously.

The invention is also directed to devices for combining at least two fluid reactants, comprising at least a first and a second conduit for delivering separate fluid reactants; a reaction chamber in fluid communication with said first and second conduits, wherein the reaction chamber allows for the mixing of the at least two fluid reactants; and a reaction chamber port allowing for the passage of the mixed at least two fluid reactants to the exterior of the device. Such devices include, but are not limited to, catheters, hypodermic needles, injecting-type or infiltrating catheters, spray bottles and canisters, and irrigation bottles and bags. Such devices may be gravity-driven, pressurized, or mechanically driven.

Additional embodiments of the invention are directed to singlet oxygen generating systems comprising a) at least one peroxide source and b) at least one hypochlorite anion source, wherein the singlet oxygen generating systems comprise at least one isotope source. In some embodiments, the isotope sources are chosen from at least one of the peroxide sources and the hypochlorite anion sources. In other embodiments, the isotope sources are chosen from at least one of the peroxide sources, the hypochlorite anion sources, and nonperoxide, nonhypochlorite anion sources. In further embodiments, the isotope sources are nonperoxide, nonhypochlorite anion sources, such as the solvents of the peroxide sources and/or the hypochlorite anion sources. The nonperoxide, nonhypochlorite anion sources may be any sources other than the peroxide sources and the hypochlorite anion sources.

The invention is also directed to methods of treating a target site, comprising administering a) at least one peroxide source, b) at least one hypochlorite anion source, and c) at least one isotope source, wherein the isotope sources are chosen from at least one of the peroxide sources, the hypochlorite anion sources, and nonperoxide, nonhypochlorite anion sources. In some embodiments, the isotope sources are chosen from at least one of the peroxide sources and the hypochlorite anion sources. In other embodiments, the isotope sources are nonperoxide, nonhypochlorite anion sources. The nonperoxide, nonhypochlorite anion sources may include the solvents of the peroxide sources, the hypochlorite anion sources, or both.

In the systems and methods of the invention, the isotope sources may include radioactive isotopes, non-radioactive isotopes, or both. Isotopes may be radioactive isotopes, such as isotopes of hydrogen, isotopes of carbon, isotopes of nitrogen, isotopes of sodium, isotopes of magnesium, isotopes of phosphorus, isotopes of potassium, isotopes of calcium, isotopes of chromium, isotopes of iron, isotopes of cobalt, isotopes of nickel, isotopes of copper, isotopes of gallium, isotopes of germanium, isotopes of krypton, isotopes of rubidium, isotopes of strontium, isotopes of yttrium, isotopes of technetium, isotopes of palladium, isotopes of indium, isotopes of tin, isotopes of iodine, isotopes of xenon, isotopes of samarium, isotopes of iridium, isotopes of thallium, isotopes of bismuth, isotopes of astatine, isotopes of radium, isotopes of actinium, isotopes of americium, and isotopes of californium. Alternatively, the isotopes may be non-radioactive isotopes, such as isotopes of hydrogen, isotopes of carbon, isotopes of nitrogen, isotopes of oxygen, isotopes of magnesium, isotopes of sulfur, isotopes of chlorine, isotopes of calcium, isotopes of iron, isotopes of copper, isotopes of zinc, and isotopes of xenon. Non-radioactive isotopes of hydrogen include ²H, and non-radioactive isotopes of oxygen include ¹⁸O.

The systems and methods of the invention may be applied to target sites which are located in or on living organisms. Target sites may include warts, keratoses, papillomas, lesions, macular degenerations, dental caries, psoriases, viremias, bacteremias, fungal infections, tumors, and cancer. Target sites may also be inert areas. The invention may also be used where target sites comprise pathogens. The pathogens may be chosen from, but not limited to, bacteria, viruses, fungi, unicellular organisms, and multicellular organisms. The invention may also be used where target sites comprise abnormal growths and/or deposits, which may be chosen from, but not limited to, metastases, arteriosclerotic plaques, atherosclerotic plaques, atheromas, arterio-venous malformations, amyloid deposits, dental plaques, and inflammation sites or mutated cells.

In some embodiments, the peroxide sources, the hypochlorite anion sources, and the nonperoxide, nonhypochlorite anion sources may be administered simultaneously where the isotope sources are chosen from at least one of the peroxide sources, the hypochlorite anion sources, and the nonperoxide, nonhypochlorite anion sources. Or, at least one or all of them may be administered nonsimultaneously. In other embodiments, where the isotope sources are chosen from at least one of the peroxide sources and the hypochlorite anion sources, the peroxide sources and the hypochlorite anion sources may be administered simultaneously or nonsimultaneously.

The invention is also directed to singlet oxygen generating systems comprising at least one superoxide source as a source of singlet oxygen, wherein the systems comprise at least one isotope source. The invention is further directed to methods of treating a target site, comprising administering a) at least one superoxide source as a source of singlet oxygen and b) at least one isotope source, wherein the isotope sources are chosen from at least one of the superoxide sources and nonsuperoxide sources. The superoxide systems and methods of the invention may be applied to a target site in the same way as other embodiments of the invention.

The accompanying drawings, which are incorporated in and constitute a part of this specification, illustrate several embodiments of the invention and together with the description, serve to explain the principles of the invention.

### BRIEF DESCRIPTION OF THE DRAWINGS

It is to be understood that both the foregoing general description and the following detailed description are exemplary and explanatory only and are not restrictive of the invention, as claimed.

FIG. 1A diagrammatically illustrates a backpack unit in accordance with the present invention.

FIG. 1B diagrammatically illustrates how the device of FIG. 1A can be used to deliver streams of reactants to a target site

FIG. 1C diagrammatically illustrates an embodiment in which the distal ends of delivery conduits are held in place by a yoke mechanism.

FIG. 1D diagrammatically illustrates how spray nozzles produce a mist output that mixes at a target site.

FIG. 2A diagrammatically illustrates a spray bottle of the present invention.

FIG. 2B diagrammatically illustrates a spray bottle of the present invention, which includes a double trigger mechanism.

FIG. 3 diagrammatically illustrates a bottle with two chambers according to the present invention.

FIG. 4A diagrammatically illustrates a beveled-tip needle that may be used in the present invention.

FIG. 4B diagrammatically illustrates a closed-tip needle that may be used in the present invention.

FIG. 5 diagrammatically illustrates a cross-sectional view of a simple dual lumen catheter that may be used in the present invention.

FIG. 6 diagrammatically illustrates a cross-sectional view of a more complex catheter that may be used in the present invention.

FIG. 7 diagrammatically illustrates an apparatus that may be used for practicing the present invention.

FIG. 8 diagrammatically illustrates a dual lumen catheter with proximal and distal ports utilized in accordance with the present invention.

FIG. 9 diagrammatically illustrates a dual lumen catheter having a reaction chamber in accordance with the present invention.

FIG. 10 diagrammatically illustrates a fluid flow device of the present invention.

FIG. 11 diagrammatically illustrates a fluid flow device of the present invention.

FIG. 12 diagrammatically illustrates a fluid flow device of the present invention.

FIGS. 13A, 13B, 13C, 13D, 13E, and 13F are cross-sectional views of various fluid flow devices according to the present invention.

FIG. 14 diagrammatically illustrates a fluid flow device of the present invention.

FIGS. 15A and 15B diagrammatically illustrates different fluid flow devices of the present invention.

FIG. 16 diagrammatically illustrates a fluid flow device of the present invention.

FIG. 17A diagrammatically illustrates a hypodermic needle having a reaction chamber in accordance with the present invention.

FIG. 17B is a close-up view of the reaction chamber needle shown in FIG. 17A.

FIG. 17C diagrammatically illustrates a different embodiment of a reaction chamber needle.

FIG. 18 diagrammatically illustrates a container for delivering irrigant solutions.

FIG. 19 is a photograph of a human skin keratosis lesion approximately 1.25 cm across, prior to treatment according to this invention.

FIG. 20 is a photograph of the human skin keratosis lesion of FIG. 19 immediately after injection with 0.4 ml of 6% sodium hypochlorite.

FIG. 21 is a photograph of the human skin keratosis lesion of FIG. 19 immediately after injection with 0.4 ml of 6% sodium hypochlorite and 0.4 ml of 3% hydrogen peroxide.

FIG. 22 is a photograph of the human skin keratosis lesion of FIG. 19 three minutes after injection with 0.4 ml of 6% sodium hypochlorite and 0.4 ml of 3% hydrogen peroxide.

FIG. 23 is a photograph of the human skin keratosis lesion of FIG. 19 four hours after injection with 0.4 ml of 6% sodium hypochlorite and 0.4 ml of 3% hydrogen peroxide.

FIG. 24 is a photograph of the human skin keratosis lesion of FIG. 19 twenty-four hours after injection with 0.4 ml of 6% sodium hypochlorite and 0.4 ml of 3% hydrogen peroxide.

FIG. 25 is a photograph of the human skin keratosis lesion of FIG. 19 forty-eight hours after injection with 0.4 ml of 6% sodium hypochlorite and 0.4 ml of 3% hydrogen peroxide.

FIG. 26 is a photograph of a human skin keratosis lesion approximately 0.7 cm in diameter (lesion A), prior to treatment according to this invention.

FIG. 27 is a photograph of the human keratosis lesion A of FIG. 26 immediately after injection with 0.22 ml of 6% sodium hypochlorite and 0.44 ml of 3% hydrogen peroxide.

FIG. 28 is a photograph of the human keratosis lesion A of FIG. 26 three minutes after injection with 0.22 ml of 6% sodium hypochlorite and 0.44 ml of 3% hydrogen peroxide.

FIG. 29 is a photograph of the human keratosis lesion A of FIG. 26 twenty-four hours after injection with 0.22 ml of 6% sodium hypochlorite and 0.44 ml of 3% hydrogen peroxide.

FIG. 30 is a photograph of the human keratosis lesion A of FIG. 26 eight days after injection with 0.22 ml of 6% sodium hypochlorite and 0.44 ml of 3% hydrogen peroxide.

FIG. 31 is a photograph of the human keratosis lesion A of FIG. 26 thirteen days after injection with 0.22 ml of 6% sodium hypochlorite and 0.44 ml of 3% hydrogen peroxide.

FIG. 32 is a photograph of the human keratosis lesion A of FIG. 26 twenty-six days after injection with 0.22 ml of 6% sodium hypochlorite and 0.44 ml of 3% hydrogen peroxide.

FIG. 33 is a photograph of the human keratosis lesion A of FIG. 26 thirty-three days after injection with 0.22 ml of 6% sodium hypochlorite and 0.44 ml of 3% hydrogen peroxide.

FIG. 34 is a photograph of a human skin keratosis lesion approximately 0.4 cm in diameter (lesion B), prior to treatment according to this invention.

FIG. 35 is a photograph of the human keratosis lesion B of FIG. 34 immediately after injection with 0.2 ml of 6% sodium hypochlorite and 0.2 ml of 3% hydrogen peroxide.

FIG. 36 is a photograph of the human keratosis lesion B of FIG. 34 three minutes after injection with 0.2 ml of 6% sodium hypochlorite and 0.2 ml of 3% hydrogen peroxide.

FIG. 37 is a photograph of the human keratosis lesion B of FIG. 34 twenty-four hours after injection with 0.2 ml of 6% sodium hypochlorite and 0.2 ml of 3% hydrogen peroxide.

FIG. 38 is a photograph of the human keratosis lesion B of FIG. 34 eight days after injection with 0.2 ml of 6% sodium hypochlorite and 0.2 ml of 3% hydrogen peroxide.

FIG. 39 is a photograph of the human keratosis lesion B of FIG. 34 thirteen days after injection with 0.2 ml of 6% sodium hypochlorite and 0.2 ml of 3% hydrogen peroxide.

FIG. 40 is a photograph of the human keratosis lesion B of FIG. 34 twenty-six days after injection with 0.2 ml of 6% sodium hypochlorite and 0.2 ml of 3% hydrogen peroxide.

FIG. 41 is a photograph of the human keratosis lesion B of FIG. 34 thirty-three days after injection with 0.2 ml of 6% sodium hypochlorite and 0.2 ml of 3% hydrogen peroxide.

FIG. 42 is a photograph of a human skin keratosis lesion approximately 0.7 cm in diameter (lesion C), prior to treatment according to this invention.

FIG. 43 is a photograph of the human keratosis lesion C of FIG. 42 immediately after injection with 0.1 ml of 6% sodium hypochlorite and 0.2 ml of 3% hydrogen peroxide.

FIG. 44 is a photograph of the human keratosis lesion C of FIG. 42 three minutes after injection with 0.1 ml of 6% sodium hypochlorite and 0.2 ml of 3% hydrogen peroxide.

FIG. 45 is a photograph of the human keratosis lesion C of FIG. 42 twenty-four hours after injection with 0.1 ml of 6% sodium hypochlorite and 0.2 ml of 3% hydrogen peroxide.

FIG. 46 is a photograph of the human keratosis lesion C of FIG. 42 eight days after injection with 0.1 ml of 6% sodium hypochlorite and 0.2 ml of 3% hydrogen peroxide.

FIG. 47 is a photograph of the human keratosis lesion C of FIG. 42 thirteen days after injection with 0.1 ml of 6% sodium hypochlorite and 0.2 ml of 3% hydrogen peroxide.

FIG. 48 is a photograph of the human keratosis lesion C of FIG. 42 twenty-six days after injection with 0.1 ml of 6% sodium hypochlorite and 0.2 ml of 3% hydrogen peroxide.

FIG. 49 is a photograph of the human keratosis lesion C of FIG. 42 thirty-three days after injection with 0.1 ml of 6% sodium hypochlorite and 0.2 ml of 3% hydrogen peroxide.

FIG. 50 is a photograph of a coronary artery blocked with sclerotic plaque, taken from a human cadaver, prior to treatment according to this invention.

FIG. 51 is a photograph of the human coronary artery of FIG. 50 after treatment with 2 ml of 6% sodium hypochlorite and 4 ml of 3% hydrogen peroxide.

FIG. 52 is a photograph of a coronary artery blocked with sclerotic plaque, taken from a human cadaver, prior to treatment according to this invention.

FIG. 53 is a photograph of the human coronary artery of FIG. 52 immediately after treatment with 2 ml of 6% sodium hypochlorite alone.

FIG. 54 is a photograph of the human coronary artery of FIG. 53 immediately after 2 ml of 3% hydrogen peroxide is added.

FIGS. 55A, 55B, and 55C are photographs, taken from different directions, of a horse having an equine squamous cell carcinoma, prior to treatment according to this invention.

FIGS. 56A, 56B, 56C and 56D are photographs, taken from different directions, of the horse of FIGS. 55A, 55B, and 55C approximately 1 month after the injections of Example 5 and prior to the injections of Example 6.

FIG. 57 diagrammatically illustrates four sets of injections for sodium hypochlorite solution and hydrogen peroxide solution against an equine squamous cell carcinoma.

### DETAILED DESCRIPTION OF THE INVENTION

This invention relates to compositions, methods, apparatuses, and systems for singlet oxygen delivery. In particular, the present invention relates to the delivery of reactants that combine to produce singlet oxygen. These reactants include a source of peroxide and a source of hypochlorite anion. The reactants are delivered in amounts designed for the production of singlet oxygen at the site of delivery. Singlet oxygen has been well studied, and numerous reviews of its chemistry and properties are available. One example is the review of singlet oxygen by Leonard I. Grossweiner, published at www.bio-laser.org.

This invention also relates to singlet oxygen generating systems including isotopes. The isotopes may be radioactive, non-radioactive, or both. The isotopes may be incorporated into the singlet oxygen producing reactants, or may be the suspending media or solution for the reactants. In the present invention, the systems can be any types of system that can produce singlet oxygen. For example, the singlet oxygen generating system can be simply three containers, such as spray bottles, that contain hydrogen peroxide, sodium hypochlorite, and isotopes, respectively. Or, the system can be a more sophisticated one that includes catheters for injecting the reactants and isotopes into a target site located in a living organism. Accordingly, any specific types of system, either simple or more sophisticated, can be chosen to accommodate the desired result.

Compositions

The basic reaction between peroxide and hypochlorite is exemplified in the reaction (I) below, in which one molecule of hydrogen peroxide is decomposed into one molecule of singlet oxygen and water.

H₂O₂+ClO⁻—¹O₂*+Cl⁻+H₂O (I)

With the readily available reactants hydrogen peroxide and sodium hypochlorite, the degradation products of the reaction are salt and water, which are nontoxic and common elements in the body, as exemplified in the reaction (Ia) below:

H₂O₂+NaOCl→¹O₂*+NaCl+H₂O (Ia)

The present invention is not limited to hydrogen peroxide, however, and the source of the hypochlorite is also not limited.

The source of peroxide may be any source of peroxide, limited only by whether the compound is acceptable for the application. For example, some peroxide sources may be more or less desirable depending on whether the singlet oxygen is to be produced within or outside a living being. When used as an injected cancer treatment, e.g., intralesionally or intravenously, toxicity of reactants would preferably be low, whereas a higher degree of toxicity might be tolerable when the singlet oxygen is used as a decontaminating agent in cleaning up a biological or chemical exposure.

Of course, it should be noted that some compounds that are toxic in high concentrations may be pharmaceutically acceptable in lower concentrations. High concentrations of reactants may be utilized to therapeutically thrombose small diameter vessels, whereas similar concentrations, when used in large diameter vessels, may not produce thromboses and may be used to dissolve arteriosclerotic plaque or treat sites of inflammation. As a basic rule, toxicity should be balanced against the potential benefit. Again, as an example, it would be undesirable if a cancer treatment were more dangerous than the cancer itself, whereas even a low level of toxicity might be welcome in exchange for the decontamination of a deadly biological or chemical agent.

For example, as noted below, metal peroxides are useful in accordance with the present invention. However, the metal counterions for the peroxides may exhibit undesirable pharmacological effects. Thus, for animal and human use, metal peroxides may be less desirable than hydrogen peroxide. Yet when the application is on an inert surface, a metal peroxide such as calcium peroxide may be advantageous.

Thus, when viewed in the context of its desired application, the source of peroxide is essentially unlimited. Specific examples include hydrogen peroxide, urea peroxide, alkyl hydroperoxides, and metal peroxides. Examples of metal peroxides include alkali metal peroxides, such as calcium peroxide. Gel forms such as carbamide peroxide may also be used. The present invention is advantageous in that readily available, and non-enzymatic, sources of peroxide may be used.

The particular source of peroxide may depend on the physical form in which it is to be delivered. For example, the peroxide may take the form of an aqueous solution if it will be delivered in liquid or mist form, or may take the form of a powder or crystal if it will be wetted before reacting. The possibilities are not limited and are determined only by the desired end use.

Also, the peroxide source may be a compound that itself forms peroxide. For example, superoxide, O₂⁻, is acted on by superoxide dismutase to produce peroxide. The superoxide itself may be a source for singlet oxygen, through its reaction with a hydroxyl radical, OH⁻. Superoxide may be used as its gas phase, which may be generated by microwave radiation of oxygen at 2450 Hz. This embodiment would be useful where intrapulmonary lesions or pathogens are treated by inhalation of a superoxide gas.

The source of hypochlorite anion is also limited only by what the particular end use dictates, weighing the disadvantages against the advantages. Thus, the source for hypochlorite anion is essentially unlimited as well. Hypochlorite may be provided by metal hypochlorites and/or hypochlorous acid. Metal hypochlorites include, but are not limited to, calcium hypochlorite, sodium hypochlorite, lithium hypochlorite, and potassium hypochlorite. Other sources of hypochlorite include those that may form or decompose to hypochlorite, such as, for example, chlorine dioxide.

Again, common sense dictates what compounds will be appropriate for particular applications. For example, in animal and human applications, lithium hypochlorite may exhibit unwanted pharmacologic effects, and sodium hypochlorite may be more appropriate. For inert surfaces, however, lithium hypochlorite may be the more desirable compound. The present invention is advantageous in that readily available, and non-enzymatic, sources of hypochlorite anion may be used.

Also, the reactants should be chosen to achieve the desired end result, and some reactants may not be appropriate. For example, Noguchi et al. recently showed that hypochlorite and hydrogen peroxide react to make singlet oxygen, whereas tert-butyl hydroperoxide and methyl linoleate hydroperoxide react with hypochlorite to give peroxyl and/or alkoxyl radicals with little formation of singlet oxygen (Noguchi, N. et al., Formation of active oxygen species and lipid peroxidation induced by hypochlorite, Arch. Biochem. Biophys. 397(2):440-47 (2002)).

The systems of the present invention may also include one or more isotopes. Briefly, isotopes are forms of the same element whose nuclei contain the same number of protons and therefore the same atomic number, but have different numbers of neutrons and accordingly different mass numbers. Isotopes of an element have nearly identical chemical properties but differ in their nuclear properties. For example, some isotopes of an element, but not others, may be radioactive. An example is hydrogen, which has three isotopes with relative masses of 1, 2, and 3. The two lighter isotopes, hydrogen (relative mass of 1) and deuterium (relative mass of 2) are stable but the third, tritium (relative mass of 3) is radioactive. The isotopes of the invention do not encompass the most abundant isotopes found in nature, which are listed on the Periodic Table of the Elements, such as hydrogen with relative mass of 1. Also, in embodiments of the invention containing isotopes, the isotopes are provided at a concentration higher than normally occur in nature. That is, naturally existing mixtures of isotopes are not intended to be the subject of the present invention, such as a small amount of deuterium present in a sample of water. The isotopes used in accordance with the present invention may include radioactive isotopes, non-radioactive isotopes, or both.

Radioactive isotopes used in conjunction with singlet oxygen generating systems include, but are not limited to, isotopes of hydrogen, isotopes of carbon, isotopes of nitrogen, isotopes of sodium, isotopes of magnesium, isotopes of phosphorus, isotopes of potassium, isotopes of calcium, isotopes of chromium, isotopes of iron, isotopes of cobalt, isotopes of nickel, isotopes of copper, isotopes of gallium, isotopes of germanium, isotopes of krypton, isotopes of rubidium, isotopes of strontium, isotopes of yttrium, isotopes of technetium, isotopes of palladium, isotopes of indium, isotopes of tin, isotopes of iodine, isotopes of xenon, isotopes of samarium, isotopes of iridium, isotopes of thallium, isotopes of bismuth, isotopes of astatine, isotopes of radium, isotopes of actinium, isotopes of americium, and isotopes of californium.

The singlet oxygen generating systems in accordance with the invention may include radioactive isotopes which can be used for medical diagnosis and metabolic research. These radioactive isotopes, when administered into a body, emit radiation such as electrons, photons, positrons, etc., which can be detected and analyzed from outside the body through the conventional technique, such as autoradiography, immunoassay, transmission tomography (TCT), and emission computed tomography (ECT) including single photon emission computed tomography (SPECT) and positron emission tomography (PET). In particular, the following isotopes may be included in the singlet oxygen producing systems of the invention with exemplary medical applications in parentheses: ¹³N (protein metabolism studies), ⁴⁴Ca (bone growth studies), ⁴²K, ⁵¹Cr, and ⁵⁹Fe (blood studies), ^{99m}Tc (nuclear medicine imaging), ⁸²Rb and ²⁰Tl (cardiac imaging), ¹¹¹In, ¹²³I, ⁶⁷Cu, ⁶⁷Ga, ⁶⁸Ge, ⁷⁸Kr, ⁸⁶Kr, ⁸²Sr, ⁸⁵Sr, ⁸⁹Sr, ¹²⁷Xe, and ¹³³Xe (diagnosis of various diseases), and ³H, ²⁸Mg, ¹⁴C, ⁶³Ni, and ¹³¹I (biological and medical tracers).

As an illustrative example, tritium peroxide (T₂O₂) may be used as a source of peroxide, and delivered to various target sites along with the source of hypochlorite anion, such as sodium hypochlorite, to produce singlet oxygen. Since tritium isotope (a half-life: 12.3 years) is widely used as a biomedical tracer, the target sites treated with singlet oxygen may be monitored with the conventional technique. Specifically, the progress of the treatment can be examined easily due to the tritium remaining around the target sites. On the other hand, the hydrogen peroxide solution and/or the sodium hypochlorite solution may be tritiated: tritium isotope molecules may be simply added to the solutions. Again, the target sites treated with tritium-containing solutions can be detected by the conventional technique during and after the singlet oxygen treatment.

As a further example, ²⁴NaOCl may be used as a source of hypochlorite anion, and delivered to different target sites along with hydrogen peroxide. In addition to singlet oxygen treatment, the target sites treated with ²⁴Na (a half-life: 14.96 hours) can be checked even when the reactants are administered in a small amount.

The singlet oxygen systems of the invention may include radioactive isotopes that naturally accumulate in specific organs or tissues of the body, such as iodine (the thyroid) and ^{99m}Tc (bones, heart, and other organs). When these body parts are treated with the singlet oxygen systems, their post-treatment progress can be monitored with the conventional technique. For example, if a target site is a liver or spleen, ^{99m}Tc-labeled sulfur colloid or ^{99m}Tc-labeled albumin colloid can be used along with the singlet oxygen reactant solutions to monitor the post-treatment progress of the liver or spleen.

The singlet oxygen producing systems in accordance with the invention may also include therapeutic radioactive isotopes which can be used for disease treatment, such as cancer therapy. For example, ¹²⁵I isotope molecules are included in the hydrogen peroxide solution and/or the sodium hypochlorite solution, and these ¹²⁵I-labeled solutions are delivered to target sites such as a prostate gland. Since ¹²⁵I is widely used in the treatment of prostate cancer, ¹²⁵I-containing singlet oxygen-forming systems may show synergistic effects in the cancer treatment through the activities from both the singlet oxygen and the isotope. Especially, these therapeutic radioactive isotopes may be implanted to the target sites in an encapsulated form to reduce the spreading of the isotopes into adjoining tissues. For instance, each of the hydrogen peroxide, the sodium hypochlorite, and the therapeutic isotope can be contained in separate capsules and delivered near the target site simultaneously. Or, any one or all of them can be administered nonsimultaneously.

Other examples of therapeutic radioactive isotopes, which may be included in the singlet oxygen producing systems, are: ³²P, ⁸⁹Sr, ^{117m}Sn, and ¹⁵³Sm (bone pain therapy), ⁹⁰Y (a therapeutic agent for malignant neoplasm), and ⁶⁰Co, ¹⁰³Pd, ¹³¹I, ⁹²Ir, ²¹³Bi, ²¹¹At, ²²³Ra, ²²⁴Ra, ²²⁵Ac, and ²⁵²Cf (cancer therapy). A proper therapeutic isotope can be selected depending on target size, the radiosensitivity of the target, and the property of the isotope itself, such as a half-life and a radiation strength.

The singlet oxygen producing systems of the invention may also include monoclonal antibodies and/or tissue-specific peptides labeled with the therapeutic radioactive isotopes, such as ¹²⁵I, ¹³¹I, ⁹⁰Y, and ⁶⁴Cu. For example, ⁹⁰Y-attached monoclonal antibodies are linked to capsules containing the hydrogen peroxide and the sodium hypochlorite. Then the capsules can be delivered to various types of cancer sites through the antigen-antibody reaction. As a result, the target site can be treated with both the singlet oxygen and the therapeutic isotope.

This invention may also employ radioactive isotopes that can be used in the sterilization of inert surfaces, such as medical instruments. As an example, ⁶⁰Co (a half-life: 5.3 years), widely used for sterilization, can be applied to the inert sites along with the hydrogen peroxide solution and/or the sodium hypochlorite solution. In this aspect of the invention, singlet oxygen from the reactant solutions and gamma rays from ⁶⁰Co can decontaminate the inert sites in an additive or synergistic manner.

Radioactive isotopes used in industrial processes can also be included in the singlet oxygen systems. For example, ²⁴Na and ²⁷Mg, used to find location of leaks in pipes, may be added to the hydrogen peroxide solution and/or the sodium hypochlorite solution, and these reactant solutions can be delivered to target sites, such as contaminated pipes. As a result, sterilization of pipes and spotting of leaks can be accomplished at the same time. Instead, it is also possible to locate the leaks first by applying the isotope, followed by the treatment with the singlet oxygen. Other radioactive isotopes, which may be included in the singlet oxygen producing systems, are ¹⁹²Ir and ²⁴¹Am.

The singlet oxygen generating systems of the invention may also include non-radioactive isotopes, such as isotopes of hydrogen, isotopes of carbon, isotopes of nitrogen, isotopes of oxygen, isotopes of magnesium, isotopes of sulfur, isotopes of chlorine, isotopes of calcium, isotopes of iron, isotopes of copper, isotopes of zinc, and isotopes of xenon. They are stable isotopes that do not spontaneously decay or emit radiation. As a result, they are advantageous in having little or no physiological risks.

The systems in accordance with the invention may include non-radioactive isotopes which can be used for biomedical metabolic research. These non-radioactive isotopes are measured by mass spectrometric or magnetic resonance technique, such as magnetic resonance imaging (MRI). For example, compounds labeled with stable isotopes, such as 1-¹³C-labeled glucose, can be administered along with the hydrogen peroxide and the sodium hypochlorite to a target site having atherosclerotic plaques. The change of the plaques can be visibly examined by the MRI, and additional administration of the hydrogen peroxide and the sodium hypochlorite can be made depending on the MRI results. As another example, water labeled with both deuterium (²H) and oxygen-18 (¹⁸O) can be administered to a target site along with the hydrogen peroxide and the sodium hypochlorite, thereby allowing the post-treatment metabolism of the target site to be monitored by mass spectrometry. Other non-radioactive isotopes, which can be used in conjunction with the reactants of the invention, are: ¹⁵N, ²⁵Mg, ²⁶Mg, ³³S, ³⁴S, ³⁵Cl, ³⁷Cl, ⁴²Ca, ⁴⁴Ca, ⁵⁴Fe, ⁵⁷Fe, ⁵⁸Fe, ⁶³Cu, ⁶⁵Cu, ⁶⁴Zn, ⁶⁷Zn, ⁶⁸Zn, ⁷⁰Zn, and ¹²⁹Xe. These isotopes are listed only for the purpose of illustration without limiting the scope of the present invention as a result.

Among the non-radioactive isotopes of the present invention, deuterium is a stable, non-radioactive isotope of ordinary hydrogen and is also referred to as “heavy hydrogen.” Pure deuterium is a colorless, odorless, non-toxic, diatomic, flammable gas. Deuterium occurs naturally in all hydrogen compounds with an abundance of 0.015 percent. Approximately one of every six thousand drops of water is actually deuterium oxide and the overall deuterium to hydrogen ratio on earth is 1:6600. Its compounds are physically almost identical with the corresponding hydrogen compounds, but chemical reactions are usually slower and their spectra are different.

A water molecule in which the hydrogen has been replaced with deuterium is called deuterium oxide (D₂O) or “heavy water” and has been used in particle accelerators. Deuterium oxide is a clear liquid with a boiling point at 101.4° C., a melting point at 4° C. and a specific gravity of 1.107. Transport information indicates that it is non-toxic for air, sea and road freight. Deuterium oxide looks and tastes similar to water, but is more viscous than water and pure heavy water does not support animal life.

The low toxicity of D₂O in mammals has justified its wide usage in measuring water spaces in humans and other animals (Kushner, D. J., et al., Pharmacological uses and perspectives of heavy water and denatured compounds, Can. J. Physiol. Pharmacol. 77(2):79-88 (1999)). In fact, D₂O has proven to be safe even in studies on infants (Davies, P. S., Stable isotopes and bioelectrical impedance for measuring body composition in infants born small for gestational age, Horm. Res. 48 Supp.1:50-55 (1997)). The use of deuterium on humans is described in U.S. Pat. No. 5,223,269. Therefore, the low toxicity of D₂O to humans is well established. Also, according to the Airgas Material Safety Data Sheet, 3rd rev., Apr. 7, 1999, (document #001017) prepared by Airgas Inc., pure deuterium gas has no toxicity, carcinogenicity, mutagenicity, teratogenicity, embryotoxicity, or reproductive toxicity in humans. Similarly, other non-radioactive isotopes, such as ¹³C and ¹⁸O, show very low toxicity to living organisms, even when they are administered in a relatively large amount.

In some embodiments of the invention, the hydrogen molecule of the hydrogen peroxide in the basic reaction (I) may be replaced with isotope molecules. For example, if replaced with deuterium, the basic reaction is changed into the following reaction (II):

D₂O₂+ClO⁻−¹O₂*+Cl⁻+D₂O (II)

Other isotopes, such as ¹⁸O and ³H, may also be part of the reactants. As an example, sodium hypochlorite whose oxygen molecule is replaced with non-radioactive isotope ¹⁸O (Na¹⁸OCl) may be used as a source of hypochlorite anion. As another example, tritium peroxide (T₂O₂) may be used as a source of peroxide. As a further example, sodium hypochlorite whose sodium molecule is replaced with radioactive isotope ²²Na or ²⁴Na (²²NaOCl or ²⁴NaOCl) may be used as a source of hypochlorite anion. As a further example, sodium hypochlorite having a non-radioactive chlorine isotope ³⁷Cl (NaO³⁷Cl) may also be used as a source of hypochlorite anion. Further, the source of peroxide and the hypochlorite anion may include more than one isotope, such as D₂¹⁸O₂ or ²²Na¹⁸OCl.

In another aspect of the invention, the isotopes are not part of either the hydrogen peroxide or the sodium hypochlorite. In these embodiments, the isotopes are included in the solvent of the reactants. As an example, deuterium oxide (D₂O) may be used as the solvent of the hydrogen peroxide, sodium hypochlorite, or both. Other non-radioactive isotopes may also be contained in the solution of the hydrogen peroxide and the sodium hypochlorite. For example, ¹⁸O-labeled water (H₂¹⁸O) may also be used as the solvent of the hydrogen peroxide, sodium hypochlorite, or both. Radioactive isotopes may also be included in the solvent of the reactants. For instance, tritium oxide (T₂O) may be used as the solvent of the hydrogen peroxide, sodium hypochlorite, or both. Both radioactive and non-radioactive isotopes may be included in the solvent of the reactants. For example, tritium oxide (T₂O) and deuterium oxide (D₂O) can be mixed and used as the solvent of the hydrogen peroxide and sodium hypochlorite. These isotopes may also be contained in the solvent of the reactants as a chemically or biologically modified form, such as radioactively labeled monoclonal antibodies or peptides. For example, ^{99m}Tc isotope can be added to the solution of the reactants in different chemical forms such as Na^{99m}TcO₄ and ^{99m}Tc-HMPAO (hexa methyl propylene amine oxide). As another example, non-radioactive carbon-13 isotope can be added in a compound form such as 1-¹³C-glucose.

In some embodiments, the lifetime or effect of the singlet oxygen produced by the systems of the present invention may be increased by replacing the hydrogen molecule of the hydrogen peroxide in the basic reaction (I) with isotope molecules. For example, the lifetime or effect of the singlet oxygen in the reaction (II) may be prolonged by the presence of deuterium isotopes.

In other embodiments, the lifetime or effect of the singlet oxygen may also be increased by including deuterium oxide in the solvent of the hydrogen peroxide and the sodium hypochlorite. For example, the lifetime or effect of the singlet oxygen may also be extended by using deuterium oxide (D₂O) as the solvent of the hydrogen peroxide and the hypochlorite anion of the basic reaction (I).

Generally, deuterium oxide solution (heavy water) can increase the lifetime of singlet oxygen itself (see Ameta, S. C., et al., Singlet Molecular Oxygen, Asian Journal of Chemistry Reviews 1(2): 106-124 (1990); Parker, J. G. and Stanbro, W. D., Optical Determination of the Rates of Formation and Decay of O₂ in H₂O, D₂O and Other Solvents, J. Photochem. 25: 545-547 (1984)). Deuterium oxide solution also has an anti-mitotic effect optimized between anaphase and metaphase (Lamprecht, J. et al., Mitosis Arrested by Deuterium Oxide: Light Microscopic, Immunofluorescence and Ultrastructural Characterization, Eur. J. Cell. Biol. 51(2): 303-312 (1990)). Further, deuterium oxide solution can depress the rate of tumor growth in mice (Hans Altermatt et al., Heavy Water Enhances the Antineoplastic Effect of 5-Fluoro-Uracil and Bleomycin in Nude Mice Bearing Human Carcinoma, Int. J. Cancer 45(3): 475-480 (1990)). Moreover, oxidative damage to cancer cells by reactive oxygen species can be enhanced by deuteration. (Kamat, J. P. and Devasagayam, T. P., Oxidative damage to mitochondria in normal and cancer tissues, and its modulation, Toxicology 155(1-3):73-82 (2000)). Therefore, deuterium isotope can extend the lifetime or effect of the singlet oxygen produced by the reactants of the invention while administered as the solvent of the reactants (D₂O) or the source of the peroxide (D₂O₂).

Likewise, other isotopes can show synergistic effects when they are included as the solvent of the reactants or the element of the reactants. As an example, therapeutic isotopes used in cancer therapy can enhance anti-tumor or anti-cancer effect of the singlet oxygen while they are administered along with the reactants. That is, both singlet oxygen and therapeutic isotopes can treat the target site in an additive or synergistic manner. Particularly, ³²P-labeled diphosphonate compounds, which can concentrate in cancerous bone at relatively higher levels, can be applied to bone cancer site along with the singlet oxygen reactants in order to lower pain levels rapidly. As another example, isotopes used in biomedical diagnosis can give visible images of the target site during or after the singlet oxygen treatment if they are applied to the target site along with the reactants. In this manner, the progress of the singlet oxygen treatment can be visibly checked by a diagnostic detector, such as a gamma camera, and additional administration is performed depending on the results. Specifically, radioisotopes carried in the blood, such as ^{99m}Tc pertechnate, can allow physicians to detect clogged arteries, and therefore can be used to monitor atherosclerotic or arteriosclerotic plaques during or after the singlet oxygen treatment.

In alternative embodiments, instead of being produced from the peroxide and hypochlorite reaction, singlet oxygen may be produced from superoxide, and in particular, potassium superoxide. (See, Khan, Science 168:476477 (1970)). In still other embodiments, singlet oxygen may be produced using radiofrequency, as described by Corey & Taylor (J. Amer. Chem. Soc. 86: 3881 (1964)). Accordingly, the system of the present invention may include superoxide molecules as a source of singlet oxygen, and isotopes. For example, deuterium oxide (D₂O) solution containing potassium superoxide can be administered to a target site having tumor or cancer. As another example, ⁴²K superoxide (the half-life of ⁴²K: 12.36 hours) can be injected to localize and treat a target site having brain tumors. Other isotopes, radioactive or non-radioactive, can also be administered to target sites along with the superoxide.

The reactants, peroxide and hypochlorite anion, may be delivered in whatever physical form is desirable for the user. For example, nebulized, atomized, aerosolized, and in solutions, gels, solids, semi-solids, pastes, powders, mists, sprays, foams, suppositories, emulsions, lotions, douches, flushing solutions, sponges, troches, and other forms may be produced. The reactants may be delivered in sustained release form. For example, two separate solid or semi-solid implants, each with a different reactant, may be implanted in the locality of the tumor, to release their contents for a sustained reaction. As another example, a solid and liquid may be utilized; the solid form of one reactant held in place such that the liquid form of the other reactant flows over or comes in contact with the solid, thereby producing singlet oxygen. Routes of administration may be varied as well. For example, the compositions may be injected intravenously, intradermally, intraperitoneally, intrathecally, subcutaneously, and/or subdermally. The reactants may also be applied cutaneously using saturated cotton balls, swabs, pledgets, swatches, etc.

A single implant, divided into two solid reactant halves, may be administered. In another alternative, fluid reactants are injected, but designed to harden once in place, thereby releasing reactant over a period of time. In another alternative, separate granules of the reactants are interspersed in a single tablet or capsule, only to react upon dissolution.

The flexibility of form is also advantageous in applications outside of a living body. For example, a decontaminant foam may be prepared that includes a source of peroxide and hypochlorite in sustained release form, so that the reactants may be released over a period of time, increasing the effectiveness of the decontamination. Obviously, the choice is determined by the end use, and the disadvantages and advantages of the particular delivery route will be considered in making the choice. The use of isotopes, either as the primary element of reactants or as the solvent of reactants, would allow checking and monitoring areas that have been treated with the singlet oxygen generating system of the invention in cases of biological or chemical warfare. In addition, gaseous form of the reactants may be used in treating lung cancer or sites of biological and chemical warfare contamination.

Solutions have the advantage of rapid mixing, but may be more difficult to work with. Gels may not mix as quickly, but may be handled more easily. Solutions may have the disadvantage of more rapid dissipation into the surrounding area, as opposed to gels or pastes, which tend not to rapidly diffuse or dissipate. Depending on the desired result, it may be advantageous to deliver a gel with one reactant into the delivery site, followed by delivery of a liquid. The opposite may be desirable under other circumstances. Obviously, the particular combinations are left to the practitioner, and can be easily determined and then modified as necessary.

Isotopes used in conjunction with the reactants can also have flexible forms: solid, liquid or gaseous form. For example, deuterium oxide and ¹⁸O-labeled water are used in liquid form. On the other hand, 1-¹³C-labeled glucose or calcium-42 carbonate (⁴²CaCO₃) are used in powder form. Also, isotopes can be attached to other chemical or biological materials, such as monoclonal antibodies. Further, encapsulated isotopes can be implanted to various target sites to localize the therapeutic effect of isotopes to the target sites. Again, their forms can be selected to achieve the desired result.

Similarly, the order of delivery is left to the practitioner. The peroxide source may be delivered first, followed by the hypochlorite anion source, or vice versa. It should be noted that because living organisms often have mechanisms, e.g., catalase or peroxidase, for destroying peroxide, it may be desirable to deliver the hypochlorite first, to avoid unwanted destruction of the peroxide reactant prior to the reaction.

The reactants may be delivered from separate reservoirs, combining only at the target site. This embodiment may be advantageous if an immediate reaction is desirable, and in such case, a solution of each reactant could be used. As it is believed that the lifetime of singlet oxygen is only about 50 nanoseconds, it may be an advantage to keep reactants from reacting until in place at the target site.

Isotopes may be administered in any combinations of order with regard to the reactants. For example, deuterium peroxide (D₂O₂) and sodium hypochlorite (NaOCl) may be delivered at the same time. Instead, deuterium peroxide may be administered first, followed by sodium hypochlorite, or vice versa. As another example, a target site can be treated with deuterium oxide (heavy water) solution including hydrogen peroxide (H₂O₂) and sodium hypochlorite (NaOCl). Alternatively, a target site can be irrigated with heavy water, followed by treatment with hydrogen peroxide and sodium hypochlorite. In that case where the target site is treated with heavy water first, it is possible to administer the hydrogen peroxide next, followed by the sodium hypochlorite last, or vice versa, instead of administering the hydrogen peroxide and the sodium hypochlorite simultaneously. Therefore, isotopes, the peroxide, and the hypochlorite anion can be delivered simultaneously or nonsimultaneously to a target site in any order. In this context, nonsimultaneous administration means that at least one of the reactants is administered separately. In other words, any one or all of the reactants can be administered separately during the nonsimultaneous administration. Therefore, nonsimultaneous administration encompasses situations in which some reactants are delivered simultaneously, but at least one reactant is delivered separately.

In determining the appropriate dose, effectiveness is balanced against toxicity. Hydrogen peroxide has been administered to animals in the past, and published studies provide much in the way of guidance for avoiding toxic doses in internal administration. The following discussion is intended to enlighten that aspect of the invention.

One of the first reported cases of infusion of an intravenous hydrogen peroxide solution was by T. H. Oliver, in which he described a high rate of success in treating influenzal pneumonia. (Oliver, T. H., et al., Influenzal pneumonia: the intravenous injection of hydrogen peroxide, Lancet 1:432-433 (1920)). But it is the late Dr. C. H. Farr who should be credited with the more recent advancements in this area. (Farr, C. H., Rapid Recovery from Type A/Shanghai Influenza Treated with Intravenous Hydrogen Peroxide, OnLine J. of Alt. Med. Vol. 1, Bio-Oxidative Medicine Section (1993); Charles H. Farr, M.D., Ph.D., The Therapeutic Use of Intravenous Hydrogen Peroxide (Monograph), Genesis Medical Center, Oklahoma City, Okla. 73139 (Jan. 1987); Dormandy, T. L., In Praise of Peroxidation, Lancet II:1126 (1988)). His guidelines for preparation for intravenous peroxide solutions are as follows:

Dr. Farr begins with 30% hydrogen peroxide of USP food or cosmetic grade. Thirty percent hydrogen peroxide is a powerful oxidizer and should be handled with extreme caution.

The 30% solution is diluted with equal amounts of sterile distilled water to make a 15% stock solution. The stock solution is passed through a Millipore 0.22 μm medium flow filter for sterilization and removal of particulate matter. The stock solution is stored in 100 ml sterile containers and kept refrigerated for future use.

The infusion solutions are then prepared using sterile 5% dextrose in water. The addition of 0.25 ml of sterile 15% hydrogen peroxide stock solution to each 100 ml of carrier solution produces a 0.0375% concentration that is finally used for the intravenous infusions.

Also, it should be noted that the action of inspired oxygen with hemoglobin can produce superoxide, which when acted upon by the enzyme superoxide dismutase, yields peroxide. In this ongoing bodily process, this hydrogen peroxide is reduced by the enzyme catalase to oxygen and water. Thus, there exists a biofeedback system between catalase activity, inspired oxygen and hydrogen peroxide levels. It has been reported that the concentration of hydrogen peroxide in human blood plasma is up to about 35 μM (see generally Halliwell, B. et al., Hydrogen Peroxide in the Human Body, FEBS Letters 486: 10-13 (2000)). These peroxide concentrations are helpful in determining the lower limit of solution concentrations for intravenous singlet oxygen perfusion/infusion by the present method (For additional information, reference is made to Finney, J. W., et al., Removal of cholesterol and other lipids from experimental animals and human atheromatous arteries by dilute hydrogen peroxide, Angiology 17:223-228 (1966); Lebedev, L. V., et al., Regional oxygenation in the treatment of severe destructive forms of obliterating diseases of the extremity arteries, Vestn Khir Im II Grek 132(3):85-88 (1984)). Additional safety guidelines for hydrogen peroxide can be found on the Internet at http://www.ee.surrey.ac.uk/ssc/h202conf/dmattie.html.

Obviously, the concentrations of the reactants may be varied. The reactants generally are used in amounts sufficient to generate an effective amount of singlet oxygen at the target site. It has been suggested that 10¹⁰ molecules of singlet oxygen are necessary to kill a single cell. (Oseroff et al., Antibody-targeted photolysis: selective photodestruction of human T-cell leukemia cells using monoclonal antibody-chlorin e6 conjugates, PNAS U.S.A. Vol. 83(22): 8744-8748 (1986)). Clearly, the concentrations may be adjusted as needed, and one of skill in the art may determine which concentrations will be most effective for the particular application. As a nonlimiting example, the concentrations of the peroxide source may range from nanomolar to molar, e.g., from as low as 0.1 nanomolar to as high as 10 molar. Ten molar is a little higher than 30% hydrogen peroxide, and while concentrations higher than 10M may be used, they should be used with great care due to the strong reactivity of peroxide.

The peroxide and hypochlorite may be present in equimolar amounts, and an equimolar ratio is advantageous in allowing a complete reaction. Thus, the concentration of hypochlorite may range from as low as 0.1 nanomolar to as high as 10 molar. From a practical standpoint for many applications, however, the concentration of hypochlorite, and similarly, peroxide, will be less than one molar. Exceeding this concentration for either reactant may produce unwanted, or premature, oxidation from the individual reactants alone. Of course, higher concentrations may be used, but the oxidizing effect of both reactants becomes very strong, and may be limiting. The reactants may be delivered at concentrations of approximately 10 M or less, including concentrations of approximately 2 M, 1.8 M, 1.6 M, 1.4 M, 1.2 M, 1.0 M, 0.9 M, 0.8 M, 0.7 M, 0.6 M, 0.5 M, 0.4 M, 0.3 M, 0.2 M, 0.1 M, 90 mM, 80 mM, 70 mM, 60 mM, 50 mM, 40 mM, 30 mM, 20 mM, 10 mM, or less.

The concentrations of the reactants should be adjusted to achieve the desired result. In applications where toxicity may be an issue, such as in a topical antiseptic formulation, concentrations of reactants may be decreased. But where there is little danger of an adverse oxidation effect, such as in disinfecting or decontaminating an inert area, concentrations may be increased. Also, lower concentrations may be acceptable to reduce the population of a particularly susceptible pathogen, mutated, or abnormal cells, whereas higher concentrations may be needed to oxidize a chemical agent or spore form of a pathogen.

If a greater local concentration of singlet oxygen is desired, higher concentrations of reactants will be delivered, and vice versa for lower local concentrations. Similarly, if it is determined that higher concentrations are too toxic, concentrations of one or both reactants may be decreased. Within a living body, the sensitivity to the treatment may depend on the particular application, i.e. tumor, atherosclerotic plaque, or beta amyloid deposit, the size of the area being treated, the anatomical location, and on whether the singlet oxygen is used for its vasoconstrictive effect, as well as on the local blood supply.

The reactants of the present invention can be delivered into the center of the area to be treated. Or, the reactants can also be administered to the base of the lesion to be treated. Further, the reactants can be infiltrated into the area being treated, thereby saturating the lesion with the reactants.

The dose of isotopes is determined to accommodate the desired result. For example, non-radioactive isotopes can be administered in a relatively large amount since they show low toxicity to living organisms. On the other hand, dose of radioactive isotopes can be adjusted depending on the radiation strength, the half-life, the target size, the radiosensitivity of the target, and proximity of surrounding normal tissue.

Volume delivered will also vary, depending on the circumstances and preferences of the practitioner. For example, the volume desired for decontamination of a room or outdoor area will obviously be much greater. Porous materials generally require a greater volume to penetrate pores and crevices, whereas smooth materials can be treated with less. Also, the volume may be increased to treat an especially contaminated area, or may be decreased if simple cleaning is desired.

Within a living body, a small tumor mass may require only 0.5 ml of total reaction volume, whereas a large mass may need 5 ml. Volume injected may be varied as desired to optimize therapeutic effects in relation to side effects and/or end result. Other factors a medical practitioner might consider in determining dose include the aggressiveness of the tumor. For example, a benign tumor might be treated with a lower concentration, or with smaller volumes. However, a very aggressive tumor might be treated with higher concentrations, or volumes that completely invade the entire tumor tissue. These choices are left to the medical practitioner. Therapy regimes are also left to the medical practitioner. For example, a practitioner may decide to repeat administrations over a period of time ranging from hours to days to weeks or months. Alternatively, a single administration may be determined to be sufficient.

An injectable composition according to the present invention may be based on lactated Ringer's solution, dextrose in water (e.g. 5%), dextrose in normal saline, or ethanol, for example. Topical formulations may be based on a solvent, such as, for example, ethanol or dimethyl sulfoxide. Other topical formulations may be based on glycerin, aloe, lanolin, etc.

The formulations may contain other ingredients, depending on the desired use. For example, a decontaminating foam may include detergents or surfactants, or other agents that enhance the foaming effect. Other ingredients may be added for other purposes, and the composition may include surfactants, detergents, scents, colorants, viscosity-modifying agents, solvents, chelators, and pH-modifying agents. The choice of additional elements in the composition is the choice of the practitioner.

In some instances, a single administration of the composition of the invention may be sufficient to achieve a positive result. In other instances, repeated administration may be necessary. The frequency and concentration of administration will depend upon the results obtained, and may be modified as necessary. For example, where the application is a decontamination effort, samples or cultures should be taken from the contaminated area after treatments to ascertain the level of success.

Indeed, where administration is directed to inert, inorganic, or even organic surfaces, Gram staining, microscopic examination, biochemical and enzymatic tests, carbohydrate fermentation reactions, and/or gas chromatography of metabolic fermentation products, could be used to ascertain the effectiveness of treatment. These methods may be used, in particular, in testing with swabs or cultures from surfaces or aspirates, abscesses, etc.

Where the administration is to living tissues, the dosing will be determined by the response observed. For example, a single administration may be sufficient to obtain significant necrosis in the treated area. After approximately one week, the treated area should be observed to determine whether, and to what extent, treatment should be repeated. The treatment site may be “observed” by use of radiographic or endoscopic techniques, for example. A decision to treat again may be based on a reduction in the size of the treated lesion.

For external administration, observation is more easily accomplished. Necrosis should be visible in the treated area by three or four days after treatment, and a repeat administration may be useful at that time.

The singlet oxygen generating systems of the present invention is also beneficial because it is not limited by the restrictions of penetration in photodynamic therapy. Moreover, the chemical yield of singlet oxygen of the present invention can be more easily and accurately calculated.

Other details of applications and methods of delivery will be presented below in greater detail.

Applications

Because of its potent oxidizing potential, singlet oxygen is useful in a number of applications in accordance with the invention. For example, following the Sep. 11, 2001, terrorist attacks, there has been a global wakeup call to find ways to counter and/or control biological and chemical warfare agents. The present invention is ideally suited for that purpose.

Biological agents that are of great concern from a biological warfare standpoint are anthrax, brucellosis, botulism, cholera, plague, and small pox. Typical chemical agents include sarin, tabun, VX, soman, cyanide, and mustard/blistering agents. The task of securing an area of attack and of ascertaining the nature and severity of a toxic threat is usually given to the first responders, which consist of fire fighters, police officers, emergency medical personnel, and military personnel. Usually, a thorough search of the area must be a priority at the onset of an attack and in the case of a biological/chemical warfare incident, a large down-wind area must be secured and/or evacuated. And once an attack has been made, decontamination and containment becomes a problem.

Because the agents used in biological or chemical attacks are capable of being oxidized, the present invention is useful in their decontamination. In one embodiment, separate aqueous reservoirs of hydrogen peroxide and sodium hypochlorite are prepared, and the reactants are combined at the site of contamination. Pouring or spraying the separate reactants onto the affected area, either sequentially or simultaneously, is one manner in which the area may be treated. The reactants may be applied to large areas from the air with tanker planes, bucket-type helicopters, or crop dusters. A more local application may be obtained using fire trucks, street washing machines, or other similar devices filled with aqueous solutions of hydrogen peroxide and sodium hypochlorite to produce singlet oxygen which could be used to directly saturate the affected premises. The use of isotopes as the element or solvent of the reactants could be helpful in checking the area of biological or chemical warfare for effective coverage with the singlet oxygen generating system.

Even more targeted application to a contaminated area may be achieved through the use of individual backpack canisters to be worn by decontamination personnel. The backpack canisters contain separate reservoirs of peroxide and hypochlorite solutions, under pressure, which are delivered to the target area by the decontamination personnel. Canisters such as those mentioned here, and other delivery devices, are detailed below.

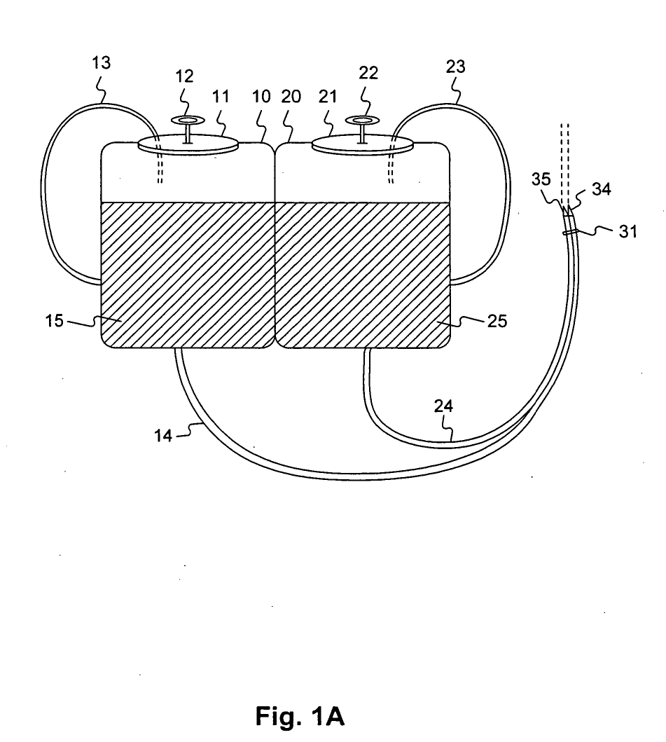

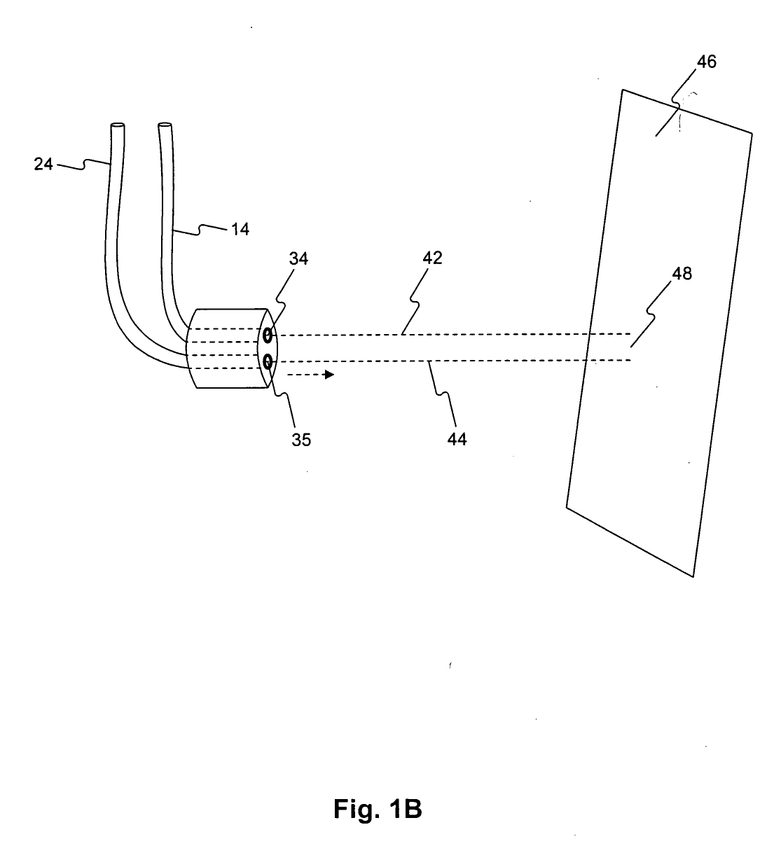

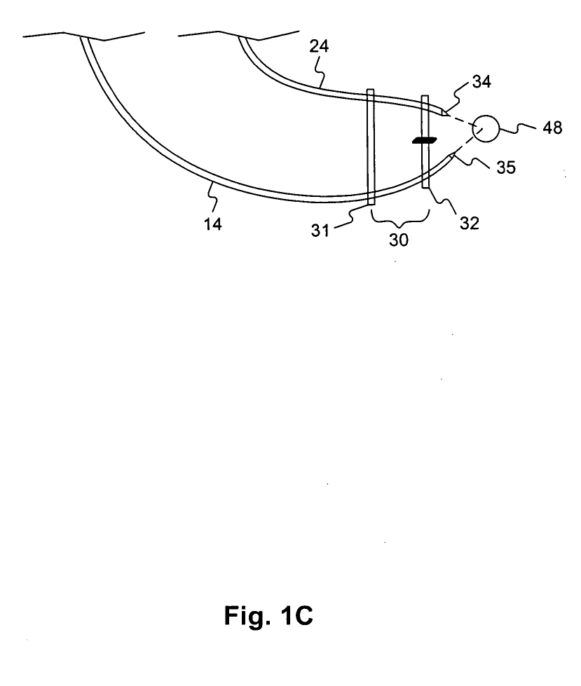

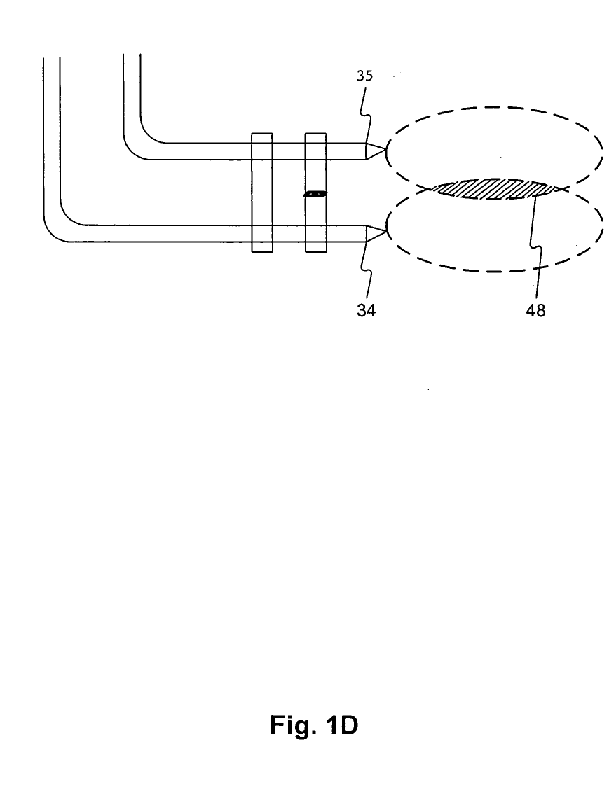

A pressurized backpack unit is shown generally in FIG. 1A. The backpack unit includes two canisters 10 and 20. In other embodiments a single divided canister serves the same purpose. Each canister includes a screw top, 11 and 21, for pouring the reactants, 15 and 25, into the respective canisters. A pump in each canister, 12 and 22, is used to introduce air into the canister to pressurize the contents. Shoulder straps, 13 and 23, secure the canister to the decontamination personnel. Separate delivery conduits, 14 and 24, deliver the pressurized contents to the target site through spray nozzles 34 and 35. In alternative embodiments, the canister is not pressurized and delivers its contents by the force of gravity. In other alternative embodiments, a reaction chamber combines the reactants prior to delivery.

In the embodiment shown in FIG. 1A the delivery conduits are joined together to deliver their respective contents in parallel to the target site, mixing on contact. A trigger mechanism 31 controls the output from the conduits. FIG. 1B shows a diagrammatic view of how the device of FIG. 1A delivers a parallel stream of reactants to a target site. Delivery conduits 14 and 24 deliver reactants through spray nozzles 34 and 35 to deliver streams of reactants 42 and 44 to a surface 46 where a reaction takes place at target site 48.

FIG. 1C shows an alternative embodiment in which the distal ends of the delivery conduits 14 and 24 are held in place by a yoke mechanism 30. The yoke includes a trigger 31 and an aiming harness 32, which is used to angle the output stream through spray nozzles 34 and 35 to a target site 48 for mixing. FIG. 1D shows a different embodiment in which the spray nozzles, 34 and 35, produce a mist output that mixes at target site 48.

In other embodiments, high pressure washing machines, generating pressures of up to 3500 psi, are used. Washer nozzles producing a fan-shaped spray, such as a 14-degree washer nozzle, may be used. Sprays or mists may be directed so as to converge at a target site some distance from the washer nozzle, such as from approximately 5 to 15 feet beyond the nozzle.

The present invention may be used where there is a need for a rapid response deployment unit located in a suspected target area. The active reagents of peroxide and sodium hypochlorite (in appropriate concentrations) can be readily and safely stored in, for example, military installations. This would give a wide distribution of these potentially life-saving reagents and since they are stable when properly stored, would make them readily available. Because these reagents are easily and economically produced, this method provides for comprehensive emergency protection from both biological and chemical warfare agents.

This application is especially desirable as compared to existing cleanup methods because: 1) the reactants are readily available; 2) the reactants are relatively inexpensive; 3) the reactants are stable in storage; 4) the reactants can be totally miscible with water, resulting in easy cleanup; 5) the reactants are quickly manufactured for additional supplies and/or in large quantities; 6) the reactants and products are primarily nontoxic in concentrations to be used; 7) excess or residual reactants are broken down by auto oxidation, ultraviolet light, and sunlight; 8) singlet oxygen is highly effective as a decontaminating agent; and 9) the reactants and product are non-mutagenic.

The present invention is also particularly applicable for use in public or industrial works. For example, where large volumes or liquids are stored, passed, or carried, growth of unwanted microorganisms, including Legionella, or even amoebic, algal, or protozoan growth, can be problematic. Specific examples include water in cooling towers, pipes, water supplies for municipalities, swimming pools, and other large stores of water, where microorganisms have a place to thrive. Other examples include main water supplies, vegetable wash water, meat process water, pasteurizers, water recycling, effluent treatment, spiral spin chillers, irrigation water, and hydroponic feed water. The potent oxidizing effect of singlet oxygen makes this invention especially useful in preventing and treating such microorganisms.

These same advantages make the present invention useful in more common applications, such as in the sterilization of hospital settings, especially operating rooms and surgical instruments, in the disinfection of bathroom floors, sinks, toilets, and tubs, and in the general cleaning of other areas in which a disinfectant or sterilant affect is desired. For example, a squirt bottle with a septum can be used to hold and simultaneously deliver aqueous solutions of peroxide and hypochlorite to a site at which singlet oxygen would be produced. A spray bottle or canister with two reservoirs could be used in this manner as well, for cleaning up restaurant or kitchen countertops and tables.

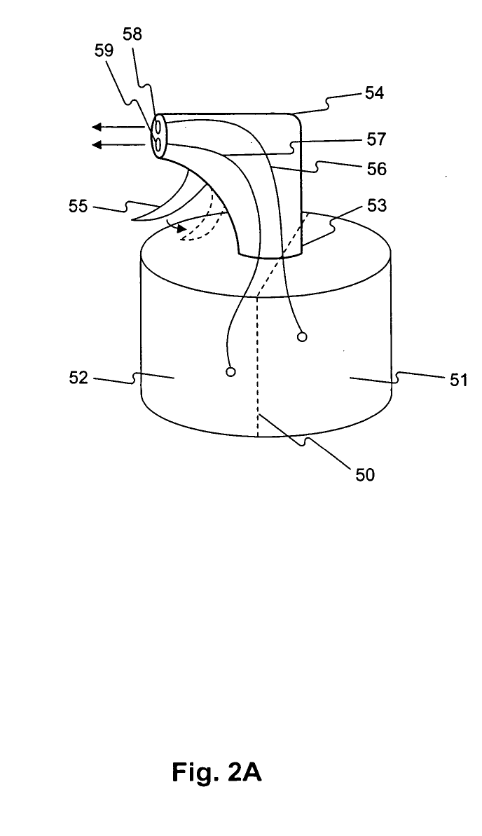

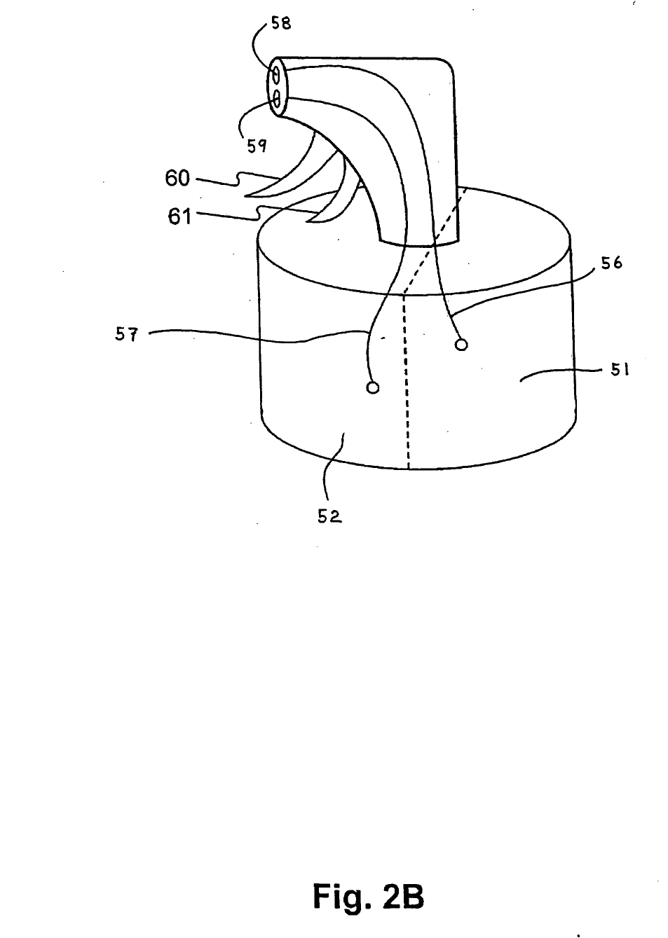

FIG. 2A diagrammatically illustrates a mechanically driven spray bottle of the present invention. In the embodiment shown, the separate peroxide and hypochlorite anion sources are kept in separate compartments, 51 and 52, of the spray bottle. A septum 50 divides one compartment from the other. A single screw top opening straddles the two compartments and a spray nozzle 54 is attached. The spray nozzle 54 includes a trigger 55. Actuating the trigger initially pulls and delivers a precise volume of a first reactant from compartment 51 through delivery conduit 56. Continued actuation of the trigger pulls an equal volume of the second reactant from compartment 52 through delivery conduit 57. In this manner, a single actuation of the trigger consecutively delivers a first and then second reactant through separate delivery ports, 58 and 59, of the nozzle. The reactants combine at the target site to produce singlet oxygen.

FIG. 2B shows an alternative trigger embodiment, which includes a double trigger mechanism. Trigger 60 pulls and delivers a precise volume of a first reactant from compartment 51 through delivery conduit 56. Actuation of trigger 61 pulls an equal volume of the second reactant from compartment 52 through delivery conduit 57. In this manner, actuating the double trigger mechanism sequentially delivers a first and then second reactant through separate delivery ports, 58 and 59, of the nozzle. The reactants combine at the target site to produce singlet oxygen.

Isotopes, either radioactive or non-radioactive, can be used in these decontamination, sterilization, and disinfection circumstances. For example, deuterium oxide solution containing the reactants can be applied to contaminated area or other inert area such as surgical instruments or kitchen countertops with enhanced singlet oxygen activity. Other isotopes can be used as described above.

Because of the nontoxic nature of the reactants and products of this invention, at an appropriate concentration, compositions of this invention may be applied directly to the skin for an antiseptic effect. For example, aqueous solutions of hydrogen peroxide and sodium hypochlorite are delivered as a mist from a spray bottle onto an area of the skin being prepared for surgery. In this embodiment, a single trigger mechanism would simultaneously deliver a mist of both reactants at the target site. In this manner, the fine mist contacts and saturates the surface area of the skin, greatly reducing the population of pathogens by the oxidizing effect of singlet oxygen.

In another embodiment, the separate reactants are applied separately. For example, separate spray or squirt bottles of peroxide and hypochlorite are made available for cleansing an area of skin to be treated. Alternatively, sponges may be used to apply the reactants, or the reactants may be supplied in pre-packaged individual “prep pads,” which are saturated in either peroxide or hypochlorite. The desired effect in these embodiments is to rid the skin of unwanted pathogens.

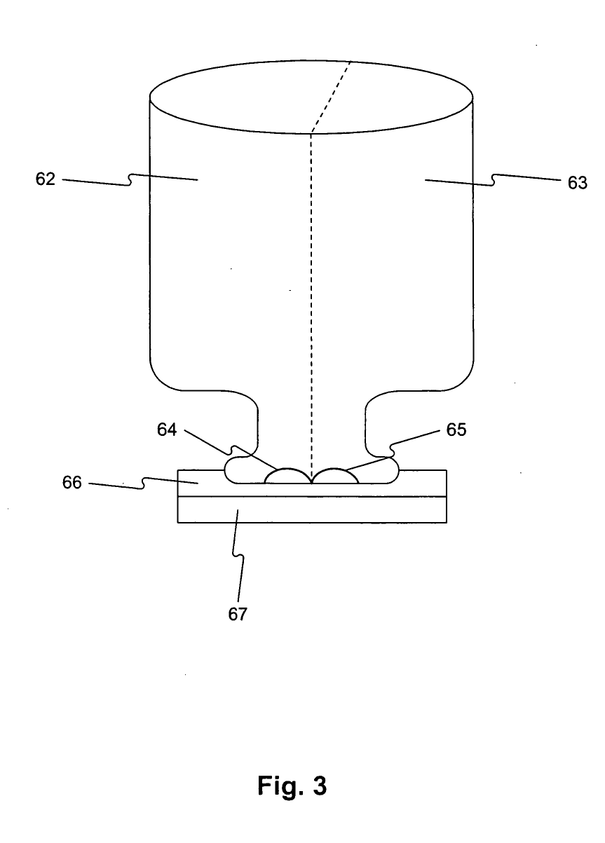

In other embodiments, the invention also finds use in topical applications as an effective exfoliant for the skin. As an exfoliant, the invention may be used to treat precancerous and cancerous skin lesions. The reactants may be supplied in two separate topical application bottles, to be applied sequentially, or in a single bottle with two chambers so the reactants are mixed during application to the skin. An example of such a bottle is illustrated in FIG. 3.

The bottle of FIG. 3 includes two chambers, 62 and 63, to contain the separate reactants. The bottle is designed to deliver by gravity or pressure the contents of the two chambers through delivery ports 64 and 65, respectively. The bottle is designed to deliver the reactants in equivalent volumes. An absorbent pad 67 is held against the delivery ports by a track 66. When the bottle is inverted or squeezed, reactants from the separate chambers are delivered simultaneously into the absorbent pad, which may then be applied topically to an area to be treated. In the embodiment shown, the used pad 67 may be removed after use, and replaced with a new pad for a new use. In alternative embodiments, the bottle is designed for a single use and the pad is made integral to the bottle.

Isotopes can also be used with the reactants for the antiseptic or exfoliant effect. As an example, deuterium peroxide (D₂O₂) and sodium hypochlorite in deuterium oxide solution (D₂O) can be administered to a skin surface or skin lesion with improved singlet oxygen production. Other isotopes can be used as described above.

The nontoxic nature of the reactants and products makes the present invention applicable in numerous applications. This nontoxic quality is especially important in applications in which the reactants are introduced into a living body to produce a reaction within it. As nonlimiting examples, cancer, atherosclerotic plaques, inflammation, or even dental plaques, may be treated in accordance with this invention. In the case of the tumor, the oxidizing effect of singlet oxygen is used to destroy cancer cells, and in atherosclerosis, the singlet oxygen oxidizes components of the plaque. It has also been indicated that vascular inflammation, which can be measured by determining blood levels of C-reactive protein (CRP), is a major causative and/or predictive factor for heart attacks, stroke or even cancer (See, e.g., Zebrack, J. S. and Anderson, J. L., Role of inflammation in Cardiovascular Disease: How to Use C-Reactive Protein in Clinical Practice, Prog. Cardiovasc. Nurs. 17(4): 174-185 (2002)). Singlet oxygen can destroy sources of inflammation, such as bacteria, fungi, viruses and cancer, and also dissolve arteriosclerotic plaques. Periodic I.V. singlet oxygen administration would act in a therapeutic and preventive manner for these conditions by dissolving plaques, destroying sources of inflammation, and killing pre-cancerous or cancerous cells.

Because the reactants are consumed in the reaction, a highly localized effect is produced. Thus, the invention is useful in local killing of cells, where more widespread destruction is undesirable, and targets for the singlet oxygen therapy include, for example, lesions, tumors, and cancer. Target sites range from the benign wart, keratoses, papillomas, to benign tumors, and even to malignant cancer.

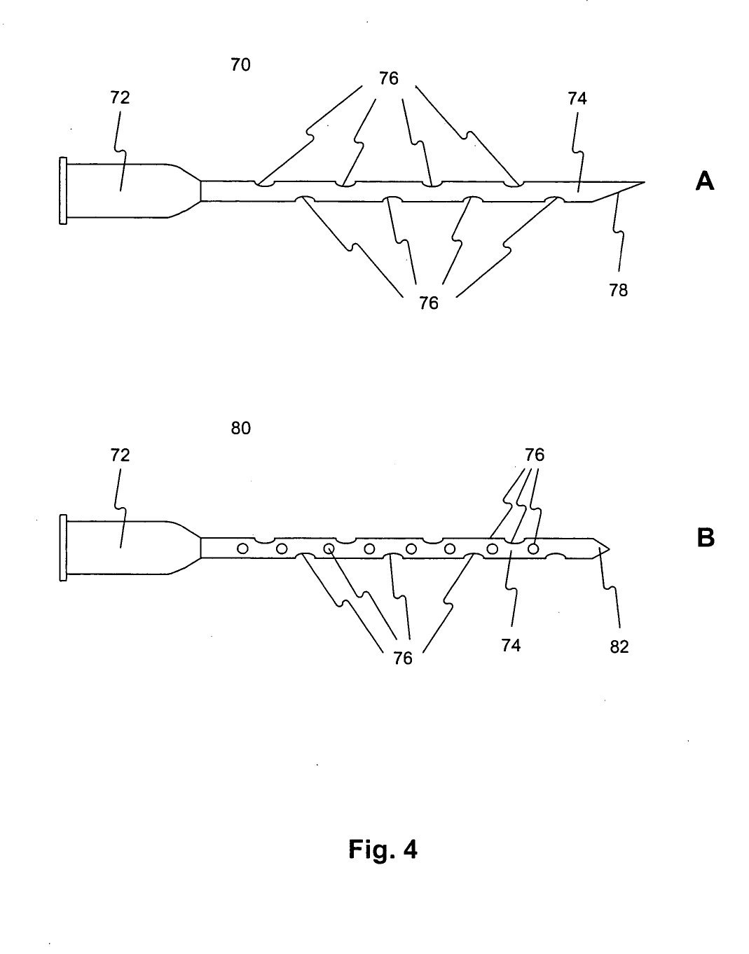

The means for delivery of the reactants to the target site may be designed to deliver the reactants sequentially or simultaneously. For sequential delivery, two syringes with needles to penetrate to the depth of the target are all that is needed. An anesthetic may be used to desensitize the area prior to treatment. The needle for delivering the reactants to the target site may be a conventional hypodermic needle, or may be a perforated hypodermic needle, as shown in FIG. 4.

Examples of perforated hypodermic needles that may be used in accordance with the present invention include the needles of FIGS. 4A and 4B, generally shown as 70 and 80. These needles include a plastic Luer-locking base 72 for attachment to a syringe. A stainless steel shaft 74 includes perforations 76 for allowing injected materials to be ejected radially from the needle. Embodiment 4A includes a beveled tip 78, whereas embodiment 4B includes a closed tip 82.

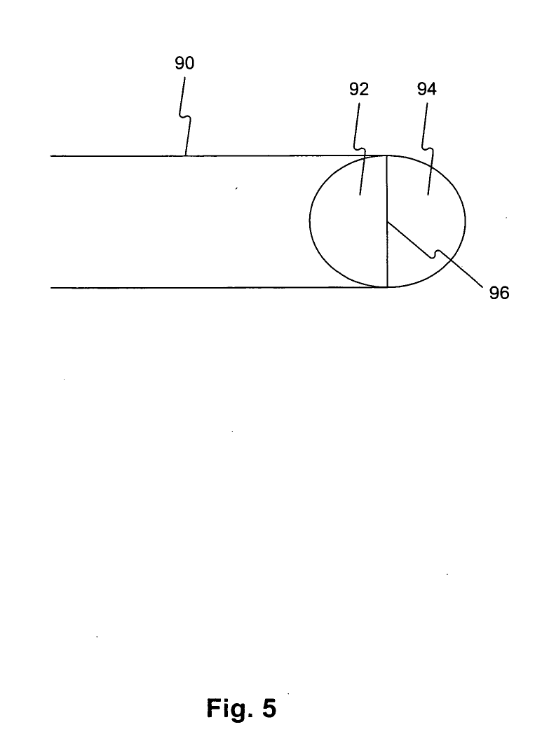

For simultaneous delivery, a dual lumen catheter may be used. FIG. 5 diagrammatically illustrates a cross-sectional view of a very simple dual lumen catheter 90 that may be used in the present invention. Catheter 90 includes a first lumen 92 for delivery of the first reactant and a second lumen 94 for delivery of the second reactant. The lumens are separated by a septum 96. Other dual-lumen catheters, such as those formed with concentric lumens may also be used.

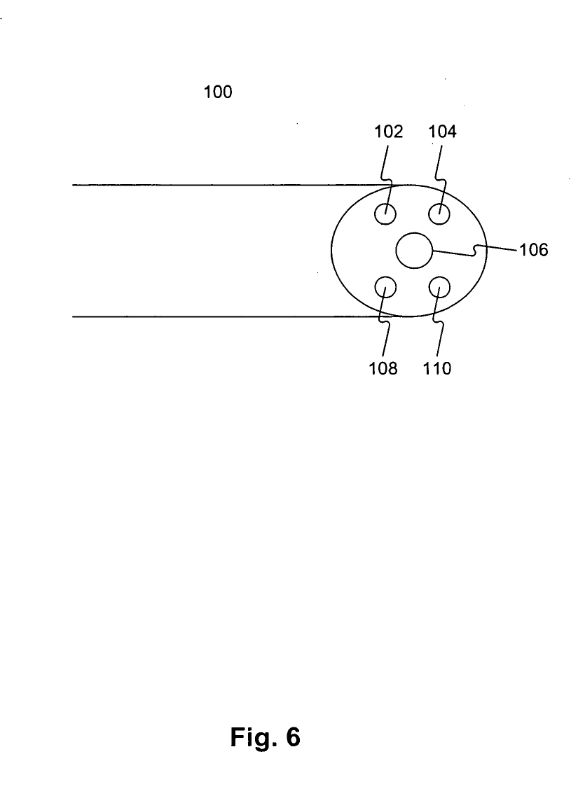

Still more sophisticated catheters may be designed or used, for example, where there is a need for an endoscope for optical guidance to the treatment site. Thus, the catheter capable of delivering two reactants may be endoscopically guided to the tumor site, where the reactants are simultaneously (or sequentially) injected. An example of such a catheter is shown in FIG. 6. A catheter might also be guided to a target site using standard radioscopic or endoscopic techniques. For example, radio opaque catheters may be placed using a guide wire and monitored using x-ray technique. This would be advantageous for lesions in the peritoneum, gut, stomach, bronchus, thoracic cavity, etc.

The catheter of FIG. 6, generally 100, includes a first lumen 102 for delivery of a first reactant and optionally the sequential delivery of a second reactant, and an optional second lumen 104 for delivery of a second reactant. A lumen 106 for an endoscope may be placed generally in the center of the catheter. Lumens for electro-cautery 108 and suction or vacuum 110, both optional, are also shown in the Figure. In other embodiments, different combinations of lumens are provided for different applications. For example, a lumen may be used for an endoscopic camera. Other applications are within the scope of the invention.

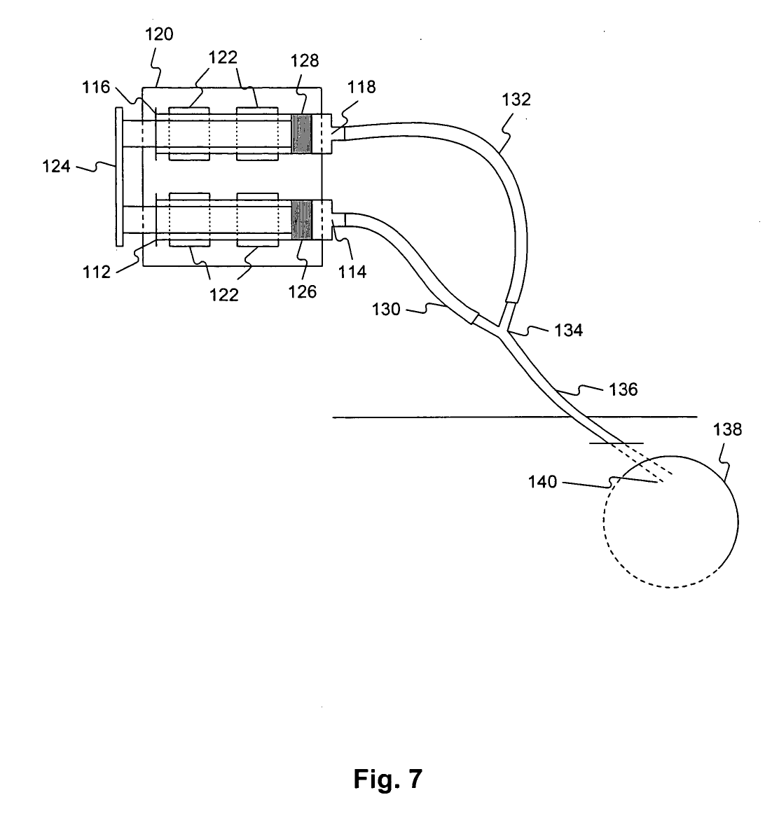

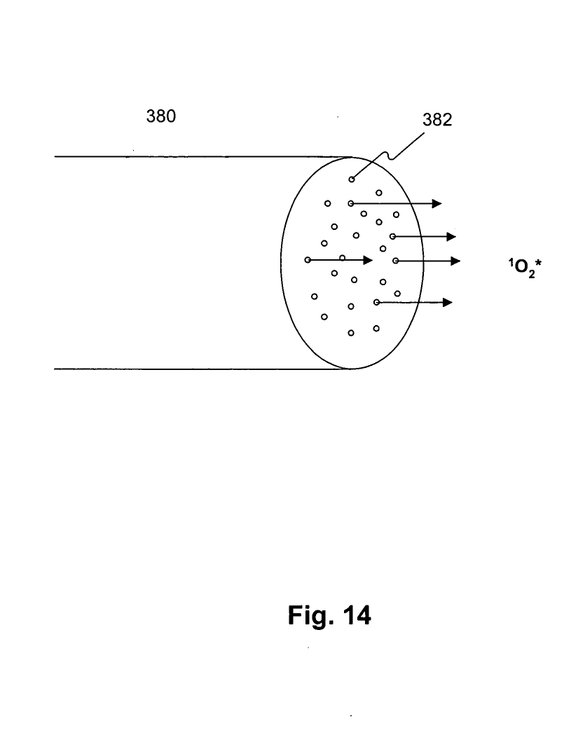

FIG. 7 diagrammatically illustrates one embodiment of the present invention in use. The system shown in FIG. 7 FIG. 14 includes a first syringe 112 for delivering a first reactant 114 and a second syringe 116 for delivering a second reactant 118. The syringes are mounted to a support plate 120 by brackets 122. An optional yoke 124 actuates first syringe plunger 126 and second syringe plunger 128 simultaneously. Upon actuation, first reactant 114 is forced into conduit 130, and second reactant 118 is forced into conduit 132. A Y-joint 134 of dual lumen catheter 136 brings together first reactant 114 and second reactant 118, without mixing. Catheter 136 is targeted into tumor 138. The reactants 114 and 118 only mix upon exit from the catheter at mixing point 140.

Although this particular embodiment has been described generally with reference to tumor treatment, the targeted delivery of the present invention provides for treatment of a wide array of conditions, including bacterial, fungicidal, viral and protozoan infections, infestations and other abnormal growths and deposits (including, for example, metastases, arterio- and atherosclerotic plaques, atheroma, arterio-venous malformations, amyloid deposits, dental plaques, HIV infection, systemic fungal infection, etc), and provides for an extremely potent vasoconstrictive effect.

Therapeutic radioactive isotopes can also be added to the reactants and delivered to a target site containing tumor or plaque. As an example, ¹³¹I can be added to the reactant solutions and administered to a thyroid gland having thyreotoxicos (Graves disease with enlargement of the thyroid gland). Synergistic effects from the singlet oxygen activity and the iodine radiation can reduce the function of the thyroid and treat the disorder. Other isotopes can be used as described above.

In another embodiment, advantage is taken of the natural fluid flow of the body to deliver reactants to the desired site. For example, the guided multi-lumen catheter is generally placed at or near the desired site or region of an infection, infestation and/or abnormal growth, and is located such that the natural direction of blood flow, whether arterial or venous or lymphatic, carries the reactants or generated singlet oxygen to the desired treatment site.

This embodiment capitalizes on the fact that the two reagents (such as hydrogen peroxide and sodium hypochlorite) are not allowed to mix or interact prior to being released at the targeted therapeutic area. With the multi-lumen catheters of the present invention, with axially spaced ports and individually separated lumens, it is possible to deliver two or more reagents to the therapeutic target and allow them to be released from different ports. The body's natural flow of arterial or venous blood will then mix the reagents such that singlet oxygen is generated and carried to and throughout the therapeutic target.

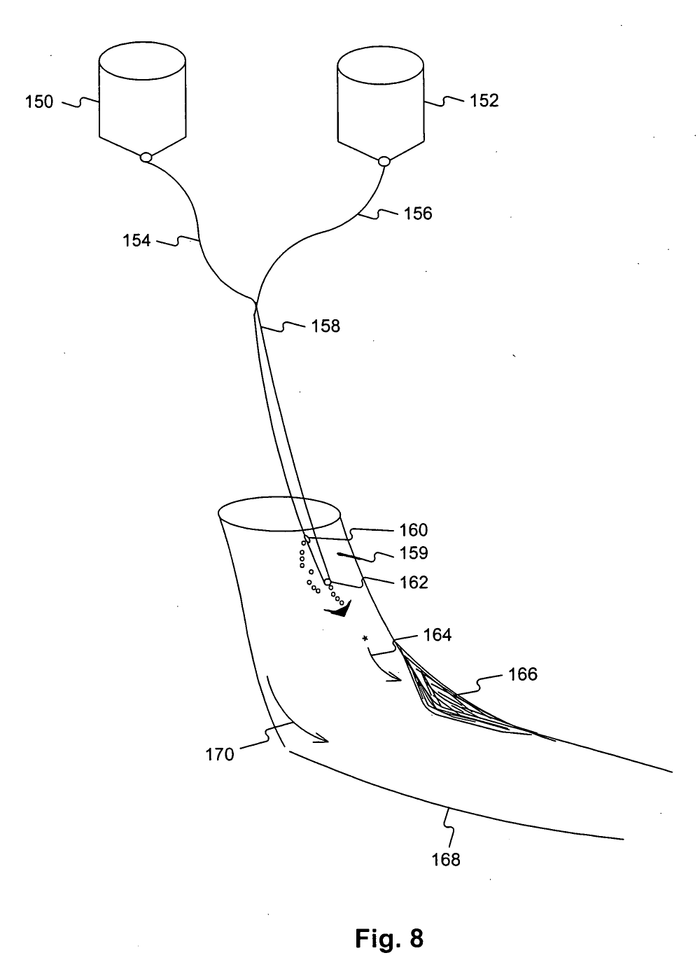

In one particular embodiment, illustrated in FIG. 8, a dual lumen catheter with proximal and distal ports is utilized. In this embodiment, separate reactant solutions are held in IV bags 150 and 152. Delivery conduits 154 and 156 carry the reactant solutions to a dual lumen catheter 158. The tip of the guided dual lumen catheter system, shown in the Figure generally as 159, is located at or near its desired treatment location within the vascular system, illustrated diagrammatically as 168. Blood flow is in the direction indicated by the arrow 170.

The tip of the dual lumen catheter 158 has a proximal port 160 from which the first reactant from bag 150 is constantly delivered. A distal port 162 located down the fluid flow 170 from the proximal port 160 constantly delivers the second reactant from bag 152. The reaction between the two reactants takes place to create a constant supply of singlet oxygen at the target site 164. In the embodiment shown, the condition to be treated is an atherosclerotic plaque 166. In alternative embodiments, this procedure may be used to treat other plaques, such as beta amyloid plaques in Alzheimer's disease.

In this model, one reactant is released from the most distal port and the other reactant from a more proximal port. This allows the first reactant to be carried by the bloodstream and mixed with the second reactant as it exits from the tip of the catheter. Consequently, singlet oxygen is perfused, infused, infiltrated or flushed through the organ or region for therapy. Since concentrations of hydrogen peroxide and sodium hypochlorite may purposely have to be kept low, the guided multi-lumen catheter can be attached to bags or bottles of these perfusate reagents and generated over time periods ranging from minutes to days of perhaps even perpetually.

Other isotopes used for blood flow diagnosis can be added to the reactant solutions in order to monitor the change of plaques during or after the singlet oxygen treatment. For example, the reactant solutions containing 400 MBq (1 mCi=37 MBq) of Na^{99m}TcO₄ can be injected to the atherosclerotic plaque and cardiovascular images of the target sites can be obtained through the SPECT technique. Other isotopes can be used as described above.

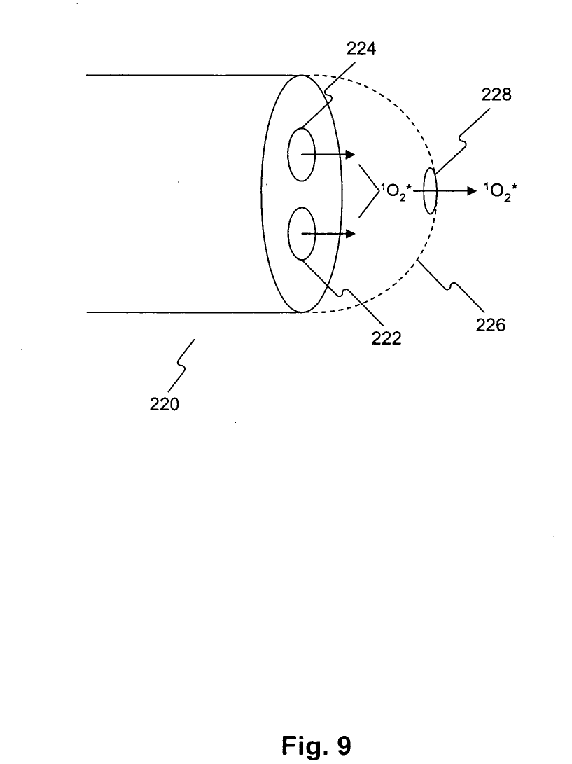

FIG. 9 diagrammatically illustrates the tip of another catheter design for use in the present invention. The catheter, shown generally as 220, is a dual-lumen type having lumens 222 and 224. An enclosed reaction chamber 226 serves as a mixing reservoir in which the reactants can react without dissipating into the surrounding blood flow. A reaction chamber port 228 serves as a point from which singlet oxygen is delivered. This design is advantageous in that it provides a reservoir in which the reactants remain at the desired concentrations, and in which the reactants are protected from breakdown by the body. The size and shape of the reaction chamber 226 can be varied such that unreacted reactants are discharged or expelled from it and allowed to be mixed by surrounding fluid flow.

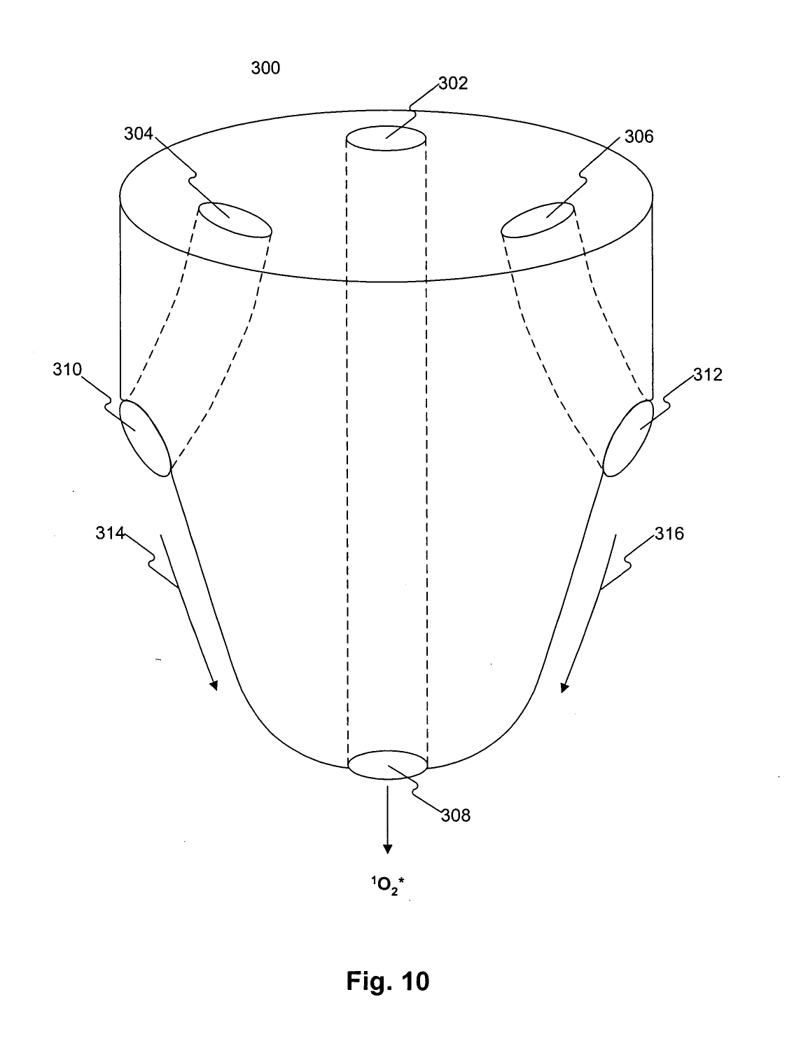

FIGS. 10 through 16 diagrammatically illustrate various fluid flow devices for delivery of excited singlet oxygen or reactants capable of producing or generating singlet oxygen. These fluid flow devices can be catheters, tubes, conduits, or any other devices that can deliver the reactants or singlet oxygen. These devices are designed to deliver singlet oxygen to a desired therapeutic site without breakdown of the reactants or singlet oxygen.

FIG. 10 diagrammatically illustrate a fluid flow device of the present invention, which can deliver singlet oxygen into the flow of arterial, venous, lymphatic, cerebro-spinal, or other bodily fluid. The fluid flow device, shown generally as 300, is a triple-lumen type catheter having lumens 302, 304, and 306. A reactant, which can be easily destroyed by catalase, peroxidase, antioxidants, etc., such as hydrogen peroxide, is delivered into the bodily fluid through an inner lumen 302. An inner delivery port 308 located at the distal end of the device 300 serves as a point from which the easily destroyable reactant is delivered into the flow of bodily fluid. Other reactants, such as sodium hypochlorite, are delivered into the fluid through outer lumens 304 and 306. Peripheral delivery ports 310 and 312 located on the side of the device 300 serve as points from which other reactants are delivered. These other reactants can flow down along the surface of the device as indicated by arrows 314 and 316. The each reactant delivered from the inner delivery port 308 and the peripheral delivery ports 310 and 312 mixes each other by the natural flow of bodily fluid. As a result, singlet oxygen is produced externally from the inner delivery port 308. This structure is advantageous in that the reactants are protected from the breakdown by the body system until they mix with other reactants to generate singlet oxygen. In this structure, flow rate of each reactant can be varied to produce singlet oxygen as needed. Similarly, this structure can be applied to double or multiple lumen catheters. It is also possible that outer lumens encircle the inner lumen approximately 360 degrees, thereby mixing the streams of two reactants at the distal end of the device.

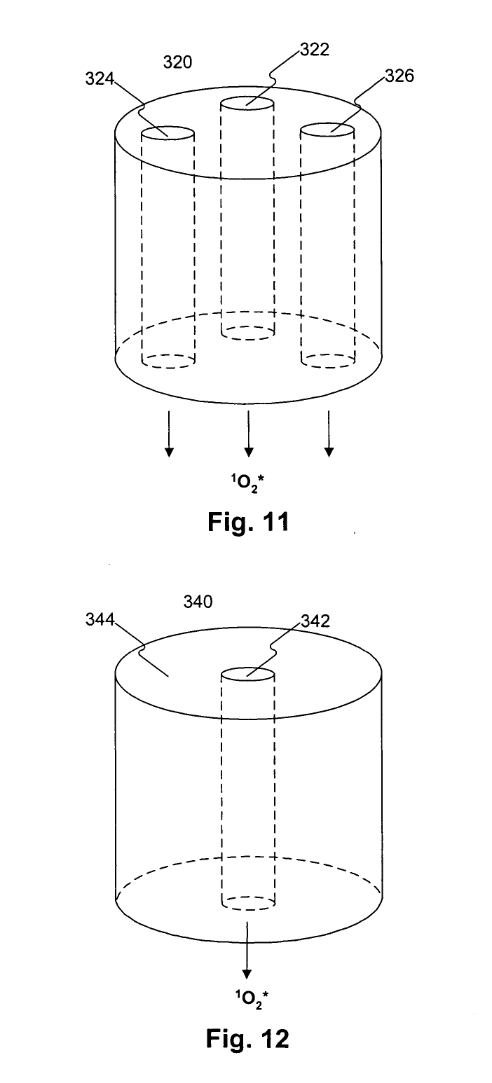

FIG. 11 diagrammatically illustrates another fluid flow device of the present invention. This device, shown as 320, is a truncated or cut-off type triple lumen catheter having three lumens 322, 324, and 326. The reactants are delivered through these lumens and mixed at the end of the lumens. Bodily fluid flow, such as blood stream, allows the reactants to react each other and generate singlet oxygen. In this structure, the combinations of reactants delivered into the lumens 322, 324, and 326 can be varied. For example, when hydrogen peroxide is delivered through the lumen 322, sodium hypochlorite is delivered through the lumens 324 and 326. It is also possible that hydrogen peroxide is delivered through the lumens 324 and 326, whereas sodium hypochlorite is delivered through the lumen 322. Likewise, the size or shape of the lumens can also be modified. In addition, this design can be applied to other double or multiple lumen catheters.

Another example of the fluid flow device 340, as shown in FIG. 12, includes concentric or annular lumens 342 and 344. A first reactant is delivered through the inner lumen 342 and a second reactant is delivered through the outer lumen 344. These first and second reactants are mixed at the ends of the lumens by the bodily fluid flow, and singlet oxygen is produced as described above.

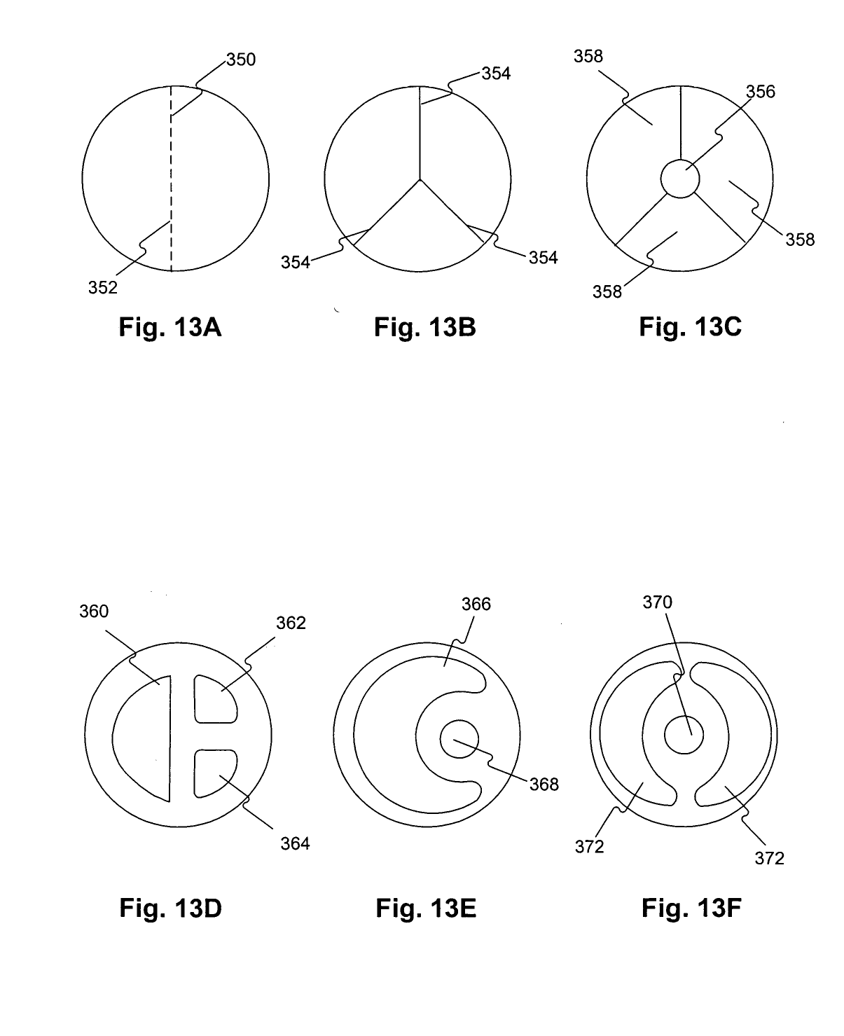

FIGS. 13A, 13B, 13C, 13D, 13E, and 13F show various cross-sectional views of different fluid flow devices that may be used in the present invention. FIG. 13A is a cross-sectional view of a double-lumen type device having two lumens connected each other through pores 352 in the inner wall 350. FIG. 13B is a cross-sectional view of a triple-lumen type device having three lumens divided by solid inner walls 354. FIG. 13C shows a cross-section of a multiple-lumen type device having one central lumen 356 and three outer surrounding lumens 358. FIG. 13D shows a cross-section of a triple-lumen type device having three lumens 360, 362, and 364. FIG. 13E shows a cross-section of another double-lumen type device in which a larger lumen 366 surrounds a small lumen 368. FIG. 13F shows a cross-section of another triple-lumen type device where a central lumen 370 is enclosed by two outer lumens 372. In these structures, the combinations of reactants delivered through the lumens can be varied. For example, in the structure of FIG. 13F, hydrogen peroxide is delivered through the central lumen 370 and sodium hypochlorite is delivered through the outer lumens 372, or vice versa.

FIG. 14 diagrammatically illustrates another example of fluid flow device, shown generally as 380. This design has many small pores and passages 382 that are connected each other. These pores and passages 382 serve as dispersing means for reactants that are delivered into the pores and passages through multiple-lumen catheters or other fluid flow devices. This design is advantageous in that the reactants are mixed uniformly through the fine pores and passages.

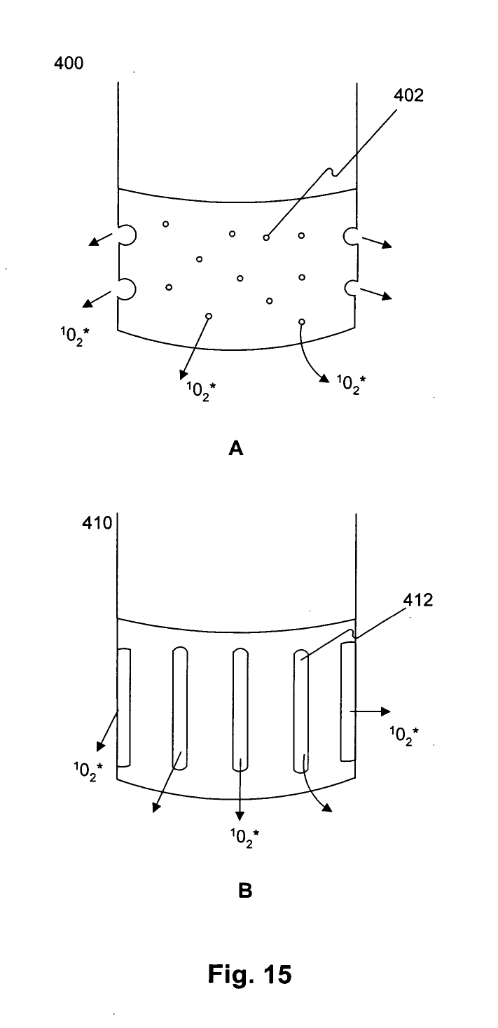

FIGS. 15A and 15B diagrammatically illustrate additional examples of fluid flow devices, shown generally as 400 and 410. These devices have multiple lateral openings 402 and 412, with or without central or distal openings. Reactants delivered to the lateral openings 402 and 412 can mix with each other and generate singlet oxygen along the sides of the devices 400 and 410. These designs are particularly advantageous in passage-type sites, such as the uterus, esophagus, and gastrointestinal tract, or in cavities, such as sinus, bladder, cerebro-spinal fluid space, intra-vascular space, vagina, nasal passage, pharynx, anus, rectum, etc. These lateral openings 402 and 412 can be in any forms of slits, slots, apertures, or other holes of various sizes, shapes, and number. It is also possible that the fluid flow device has sponge-type openings along its sides.

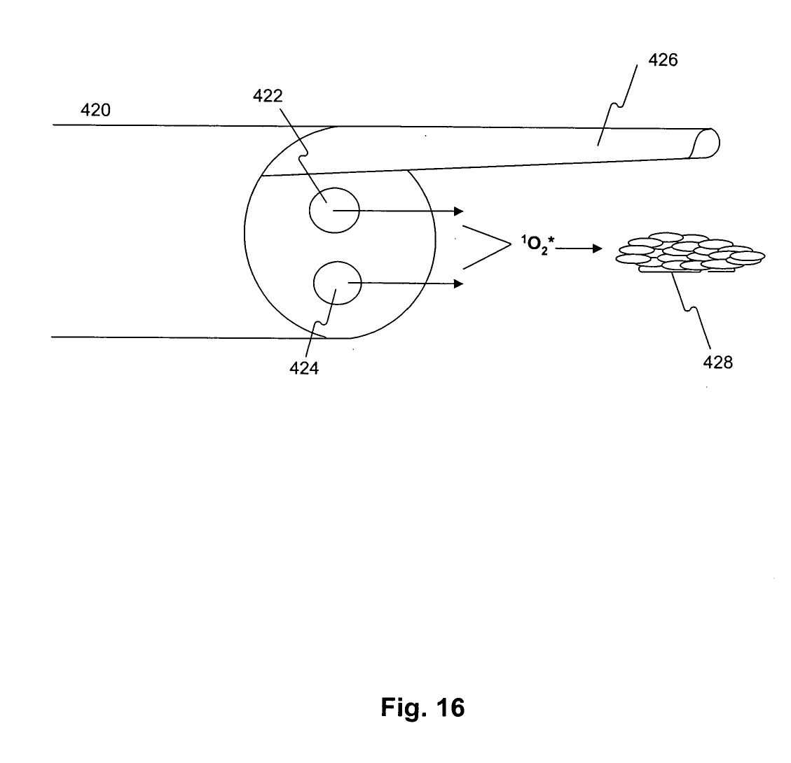

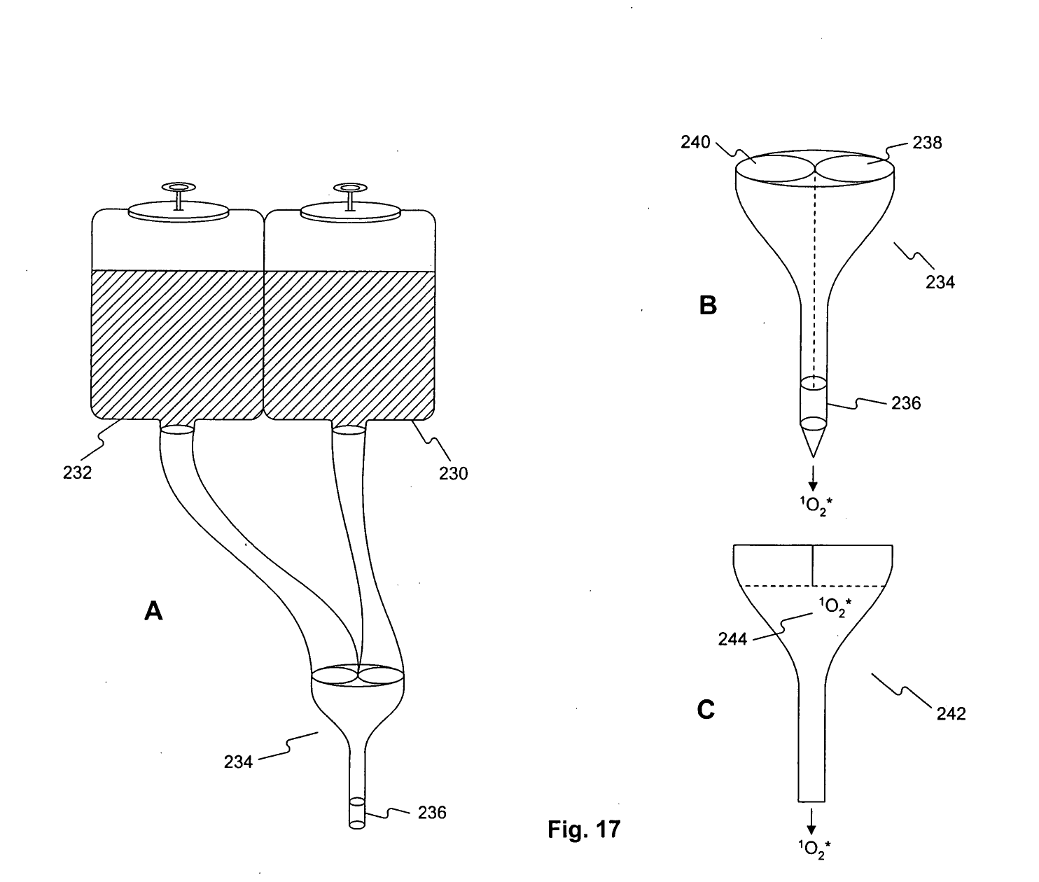

FIG. 16 diagrammatically illustrates another fluid flow device of the present invention. The device, shown generally as 420, is a double-lumen type catheter having lumens 422 and 424. The reactants delivered through the lumens 422 and 424 react each other at the distal ends of the lumens, thereby generating singlet oxygen. The generated singlet oxygen is directed or partitioned towards a desired therapeutic site 428 by a guide 426 that is attached to the distal end of the device. The desired therapeutic site 428 can be a tumor, a lesion, an ulceration, a plaque, an inflammation, etc. The guide 426 can be made of rigid, semi-rigid, or flexible materials, depending on its desired use. The number, size, and shape of the guide can also vary depending on the status of the therapeutic site and the desired purpose. This guide structure can also be applied to the above-mentioned fluid flow devices of the present invention. The guide structure may serve the role of visor or hood, compartmentalizing or directing the reactants to a desired target.

As described in the previous examples, the number, size, and shape of the lumens or walls can vary such that singlet oxygen is efficiently delivered to a desired therapeutic site, whether it be intra-arterial, intravenous, intrauterine, intra-gastric, intrathecal, etc. Also, these fluid flow devices may be combined with other devices, such as cauteries, cameras, endoscopes, biopsy apparatuses, suction apparatuses, injecting tips, fluid infusion devices, and devices for irrigant solutions, flushing solutions, neutralizing solutions, blood or blood products, medicinal substances, blood withdrawals, and light fibers or sources.

FIG. 17 diagrammatically illustrates a hypodermic needle having a reaction chamber. In the first embodiment, shown in connection with reactant reservoirs in FIG. 17A, the needle 234 is fed by separate reactant reservoirs 230 and 232. The needle prevents mixing of the reactants until reaching the reaction chamber 236. Upon reaching the reaction chamber 236, the reactants react, and singlet oxygen is produced and ejected from the needle. FIG. 17B shows a close-up view of the reaction chamber needle with separate channels 238 and 240 for keeping reactants separate. The reactants combine in the reaction chamber 236 to produce singlet oxygen.

In a second embodiment of the reaction chamber needle, shown diagrammatically in FIG. 17C, the needle 242 is much smaller. Because of the significantly reduced size, it is unnecessary to have separate channels, and the reactants flow into a central reaction chamber 244.

Devices having characteristics of both a needle and a catheter are also envisioned. For example, injecting-type or infiltrating catheters, having a sharp tip for passing through tissue, are also envisioned. These injecting-type or infiltrating catheters generally have a reaction chamber located proximally to the sharp tip. Alternatively, the reaction chamber may be formed by a sheath that is pushed over the sharp tip after the catheter has been advanced into position. In other embodiments, the sharp tip is retractable, or the sheath may protect the tip during advancement or placement.

The fact that the catheter can be guided utilizes present-day-well-known techniques to reach a wide range of body organs, systems, regions or locations. The design of the multi-lumen catheter makes it an ideal conduit to carry the two individual reactants (such as hydrogen peroxide and sodium hypochlorite) separately without mixing before arrival at the desired therapeutic site, area or region of the body.

With regard to the reaction chamber, any device that is used to deliver at least two reactants, where the reactants are to be combined before ejection from the device, may include a reaction chamber. For example, a hand-held sprayer may include a reaction chamber in its nozzle to combine reactants prior to ejection. Similarly, a backpack canister may include two delivery conduits that join together to form a nozzle that includes a reaction chamber. In other embodiments, such as in an irrigation bottle or bag, a reaction chamber is used to mix the reactants after their delivery from their separate compartments but prior to contact of the reaction mixture with the target area.

The reaction chamber could alternatively contain a solid or semi-solid form of one or both of the reactants, wherein a liquid flows over the solid or semi-solids resulting in a solution of both reactants, which then react. The liquid itself may include a reactant as well.

Returning to the discussion of catheters and other injection devices, since both peroxide and hypochlorite (and any of their chemically active derivatives or analogs) are totally miscible in water, their respective concentrations can be elegantly controlled to achieve both therapeutic levels of singlet oxygen and to keep excess singlet oxygen to a minimum. Moreover, the body possesses the necessary enzymatic systems to deal with limited excess levels of both of these physiological agents (hydrogen peroxide and sodium hypochlorite) and converts them into carbon dioxide, water, sodium chloride, and ground state oxygen. This embodiment is useful in treating organs such as the lungs, pancreas, liver, intestine, heart, stomach, brain, etc. When reagent concentrations are kept at levels to avoid air embolism, singlet oxygen can safely be delivered to these areas as needed for therapeutic purposes.

Catheter length, diameter, design, etc are determined by the regional anatomy and vasculature as regards the specific therapeutic application. Because of the short half-life of metastable singlet oxygen, this method of delivery utilizes its known activity to a great advantage. This method allows accurate and controlled delivery of singlet oxygen to a vast array of potential therapeutic sites. This makes its application essentially limitless.

Another advantage of this method is the treatment of not only a tumorous or cancerous lesion but also an entire area or region of its metastasis. This concept also extends to an infected area or even to septicemia or intravascular disseminated infections or infestations. This embodiment can be used to cleanse the blood in vivo. It allows guidance of treatment to sites of heavy growth of pathogenic organisms (bacterial, fungal, viral, including HIV, and/or protozoan) or tumorous lesions and produces elegantly controlled amounts of singlet oxygen in a safe, economical and reliable manner. Additionally, this embodiment can be an adjunct to or supplement to direct needle infiltration of singlet oxygen to desired therapeutic sites as described herein.

Because all of the body's blood circulates on a regular periodic basis, the blood will pass a common point such as the superior vena cava, the pulmonary artery, the right atrium, etc. By introducing the compositions of the present invention at that point, the entirety of the blood in the body may be exposed to the cleansing effects of the singlet oxygen. Alternatively, blood may be circulated extracorporeally, such as in dialysis, and exposed to the cleansing effects of singlet oxygen outside of the body. In this embodiment, the body's exposure to singlet oxygen is minimized, due in large part to the short half-life of the species.

In other instances where the effect on the body is to be reduced, lower concentrations and slower infusion rates may be used. Additionally, the body has mechanisms for breaking down hydrogen peroxide, and is able to utilize some hypochlorite. In any case, the short half-life of singlet oxygen is the ideal limiting factor to prevent undue toxicity build-up.

The invention may also be used as a surgical or wound irrigant. Treatment may be performed at the excision site of a tumor or abscess, or in the thoracic, peritoneal, or cranial spaces. The singlet oxygen irrigation solution would produce its beneficial bactericidal, viricidal, and tumoricidal effects as an irrigant. In such embodiments, the reactants may be provided in a single use bottle with two separate compartments. When the bottle is opened, both reactants may be discharged into the site simultaneously. Alternatively, the reactants may be supplied in separate bottles to be used together or sequentially. In addition, this invention can be used as lavage solutions, douches, rinses, and irrigants for vaginal, rectal, oral, or aural areas.

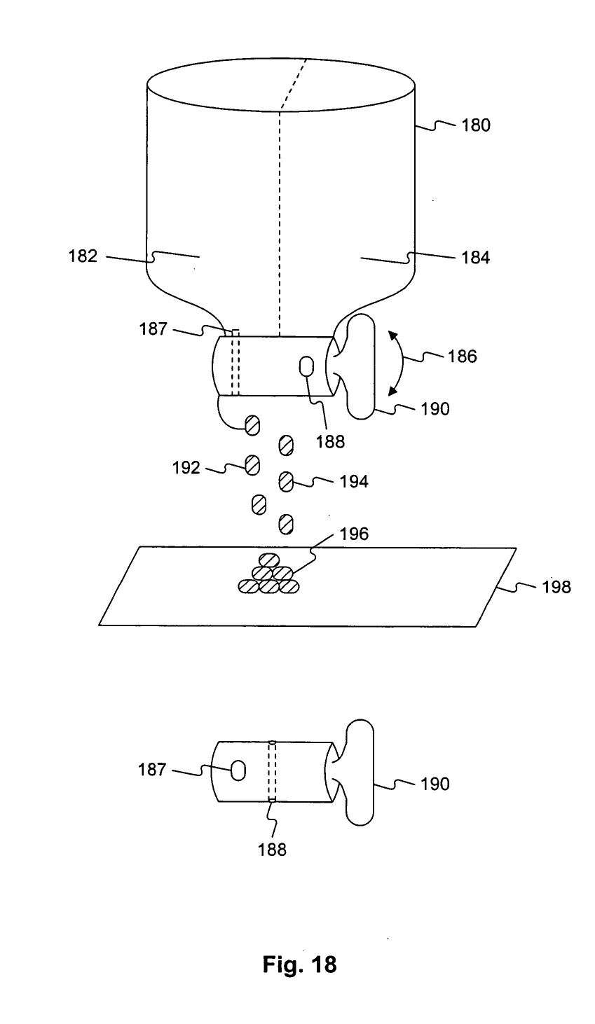

FIG. 18 illustrates a container, generally 180, which is useful in delivering irrigant solutions. Separate reactants are held in compartments 182 and 184. A stopcock 186 holds the reactants in place, and has perpendicularly placed ports 187 and 188 for alternately delivering the contents of 182 and 184. A stopcock handle 190 is turned to allow delivery of the separate reactants as drops 192 and 194, or streams, to the target site 196 of a surface 198, which may be living or inanimate. The stopcock 186 rotated by 90° is illustrated in the bottom frame of the Figure. In this embodiment, the reactants can be delivered separately without concern for premature reaction, as the stopcock is designed to separately deliver the reactants. The reactants may be delivered, for example, by pressure or gravity.

The invention is also useful as a vasoconstrictor, or in applications in which blood flow to the area is to be reduced. Hemostasis is of prime importance at an incision or operative site. The invention shows the surprising effect that singlet oxygen causes intense vasoconstriction of normal blood vessels but that the effect is later cleared from the site without harmful side effects. This effect is nearly instantaneous after administration, or at the least, occurs much more rapidly than other known vasoconstrictive methods. By applying reactants in accordance with the present invention, local blood flow may be reduced. Treatment is performed in a manner similar to that in which xylocaine and epinephrine are delivered for a vasoconstrictive effect. The invention may, therefore, find use in reducing bleeding at a surgical site or wound, or in any other situation where local vasoconstriction is desired. In addition, the invention may also include combinations with, or following, topical and local anesthetic application. Such anesthetics include but are not limited to xylocaine, novocaine, pontocaine, mepivacaine, and cocaine.

Because of the nature of the reaction, including its completeness and consumption of the reactants, this invention may be repeatedly delivered or administered without undue effects. There is no long-term or cumulative accumulation of reactants or products of the reaction. Thus, this invention is ideal where repeated administration is desirable.

The present invention also includes methods of treating target sites by administering the peroxide, the hypochlorite anion, and isotopes. As mentioned above, the isotopes contemplated include radioactive isotopes and/or non-radioactive isotopes. A more detailed description of radioactive isotopes and non-radioactive isotopes is presented above.

The isotopes can be part of the peroxide and/or the hypochlorite anion. For example, deuterium peroxide (D₂O₂) and sodium hypochlorite may be administered to the target site. As another example, tritium peroxide (T₂O₂) and sodium hypochlorite may be administered to the target site. As another example, the target site may be treated with deuterium peroxide (D₂O₂) and sodium hypochlorite whose sodium element is replaced with radioactive sodium isotope (²²NaOCl or ²⁴NaOCl). The possible substitutions are limited only by the technology and the desired use.

In other embodiments, the isotopes are not part of either the source of peroxide or the source of hypochlorite anion, but are administered or used in conjunction with the singlet oxygen-forming systems. For example, deuterium oxide (heavy water) may be used as a solvent for hydrogen peroxide (H₂O₂) and sodium hypochlorite to enhance the lifetime of the singlet oxygen. As another example, radioactive isotopes and/or non-radioactive isotopes may be added to the solvent of hydrogen peroxide and/or sodium hypochlorite as a tracer or for any other desirable purpose. As a further example, monoclonal antibodies or peptides labeled with radioactive isotopes or non-radioactive isotopes may also be included in the solvent of hydrogen peroxide and/or sodium hypochlorite.

In some embodiments, the isotopes may not only be included in the source of peroxide or the source of hypochlorite anion, but also be added in the solvent of the sources. For example, deuterium oxide (heavy water) may be used as a solvent of deuterium peroxide (D₂O₂) and/or sodium hypochlorite. Or, tritium peroxide (T₂O₂) in deuterium oxide (heavy water) can be administered with sodium hypochlorite solution. Again, the possibilities are not limited; a skilled person will recognize the desirability and utility of the use of isotopes with singlet oxygen-forming systems.

In the present methods, the target sites may be located in or on a living organism. The living organisms of the present invention include any individual living plant or animal, such as humans. In other embodiments, the target sites may be an inert area such as an operation table in a hospital or a dining table in a restaurant.

Living target sites include, but are not limited to warts, keratoses, papillomas, lesions, macular degenerations, dental caries, psoriases, viremias, bacteremias, fungal infections, tumors, and cancer. Target sites may include pathogens, such as bacteria, viruses, fungi, unicellular organisms, and multicellular organisms. Living target sites may also include abnormal growths or deposits, such as metastases, arteriosclerotic plaques, atherosclerotic plaques, atheromas, arterio-venous malformations, amyloid deposits, dental plaques, inflammation sites, or mutated cells. In the present methods, it may be possible to dissolve arteriosclerotic or atherosclerotic plaques in situ by placing an infusion catheter tip, just proximal to the occluding plaques, and infusing the singlet oxygen over the plaques. These methods may eliminate the need for extirpative surgery, photodynamic therapy, or stent placement.

The singlet oxygen and the isotopes may be used to achieve synergistic effects in the treatment of diseases, pathogens, abnormal growths, or deposits. For example, the singlet oxygen and the isotopes may greatly enhance their respective capacities when used jointly. For example, deuterium isotope can extend the lifetime of singlet oxygen, and therapeutic isotopes can synergistically or additively destroy tumor or cancer cells when administered with singlet oxygen-forming reactants.

In some embodiments, superoxide as a source of singlet oxygen and isotopes can be administered to target sites. These superoxide methods are applied to target sites in the same manner as described in the methods of peroxide and hypochlorite anion.

To summarize some embodiments of this invention, the present invention can be used, for example, in the treatment of ophthalmic conditions, such as macular degeneration; dental conditions, such as plaque and caries; dermatological conditions, such as psoriasis; gynecological conditions, such as uterine bleeding, uterine tumors, and uterine cancer; oncological conditions, such as tumors and/or cancers; cardiovascular conditions, such as arteriosclerosis and plaque formation; infective conditions, such as viremias, bacteremias, and fungal infections; and contaminated conditions, such as sterilization, disinfection, biological or chemical warfare, wound irrigation, and organic/inorganic surface cleaning.

The singlet oxygen generating system can also be applied to all of the conditions that can be treated with photodynamic therapy. These conditions include, but are not limited to, pre-cancerous and cancerous growths, dysplasia of esophagus or cervix, rheumatoid and inflammatory arthritis, psoriasis, acne, alopecia areata, port-wine stains, hair removal and/or hair growth, choroidal neovascularization, age-related macular degeneration, extracorporeal bone marrow purging and grafting, and blood-borne viruses (including HIV-1, herpes simplex virus type I/II, human cytomegalovirus, measles, and simian virus).

The following examples are intended to illustrate the invention. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result.

### EXAMPLES

### Example 1

### Treatment of Keratosis I

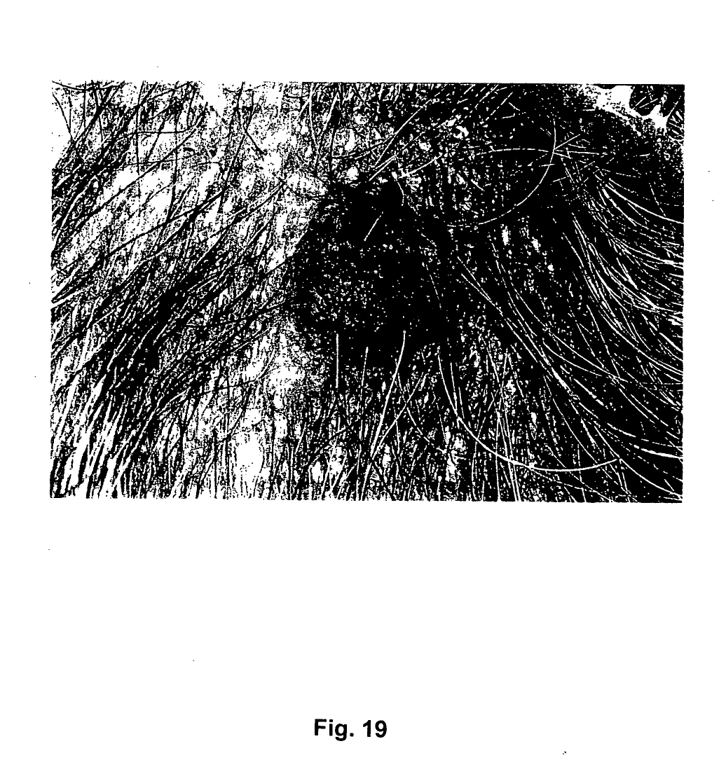

A skin keratosis lesion measuring approximately 1.25 cm in diameter was chosen as a target for treatment. The lesion was located on the left temple of a 57-year old white male. A photo of the keratosis lesion, prior to treatment, is shown in FIG. 19.

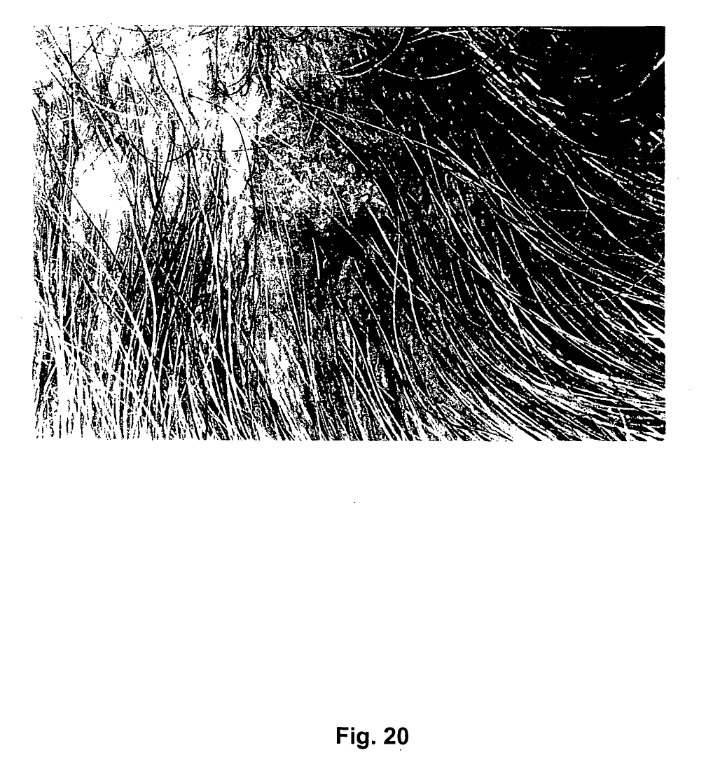

Using a 30-gauge hypodermic needle, 0.4 ml of a 6% solution of sodium hypochlorite was injected into the center of the lesion. The injection resulted in a mild burning sensation, and produced minor bleeding at the lower border of the injection site. See FIG. 20 for a photo of the area immediately following the injection.

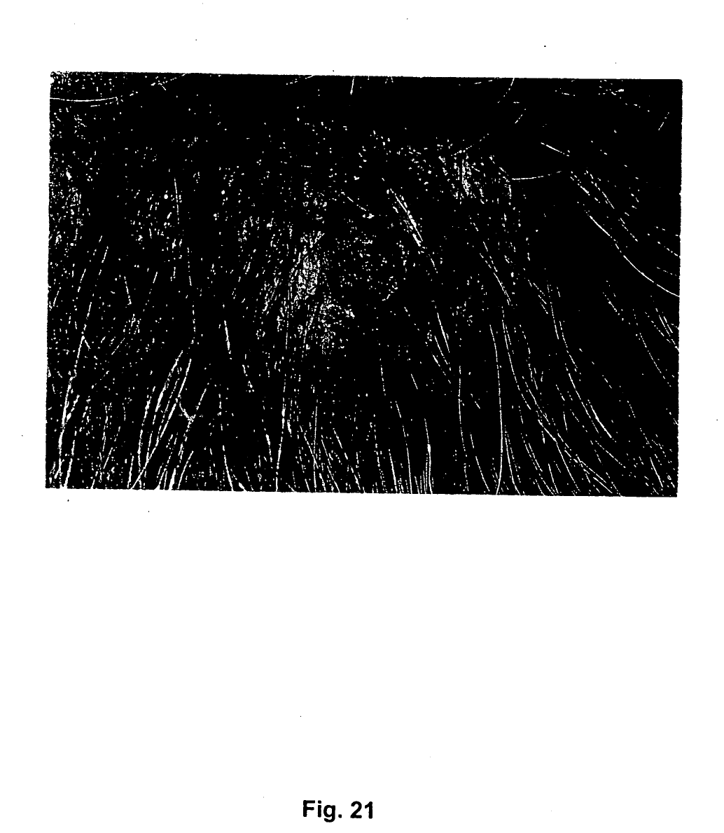

Immediately after the first injection, using a 30-gauge hypodermic needle, 0.4 ml of a 3% solution of hydrogen peroxide was injected into the center of the lesion. The injection produced foaming, or bubbling, at the surface of the lesion, and blanching of the surrounding tissue. See FIG. 21 for a photo of the area immediately following the injection.

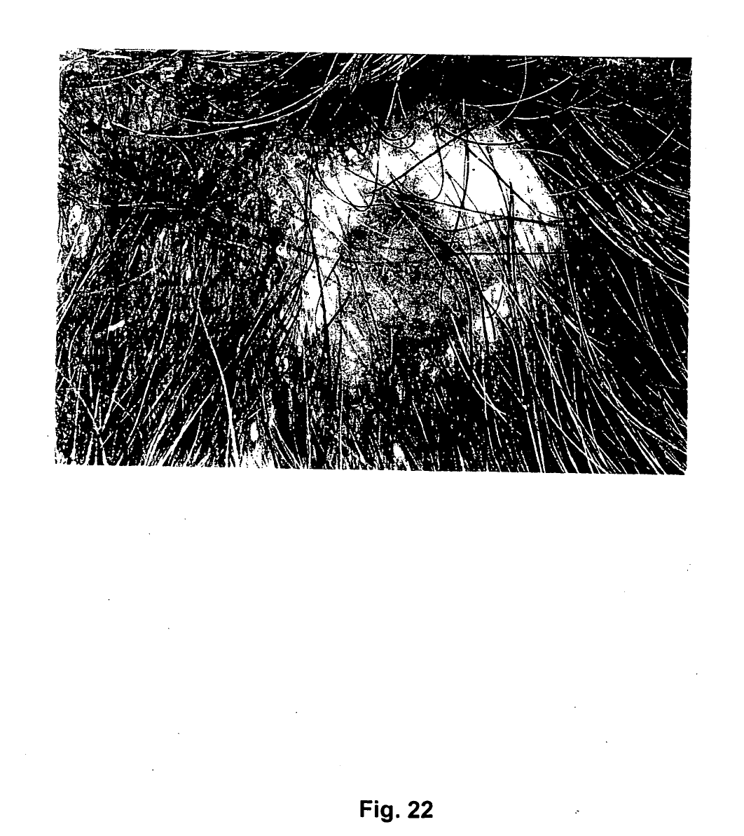

FIG. 22 shows the lesion site three minutes after injection. The marked blanching in the area surrounding the injection indicated extreme vasoconstriction. The blanched tissue was “normal” tissue surrounding the lesion.

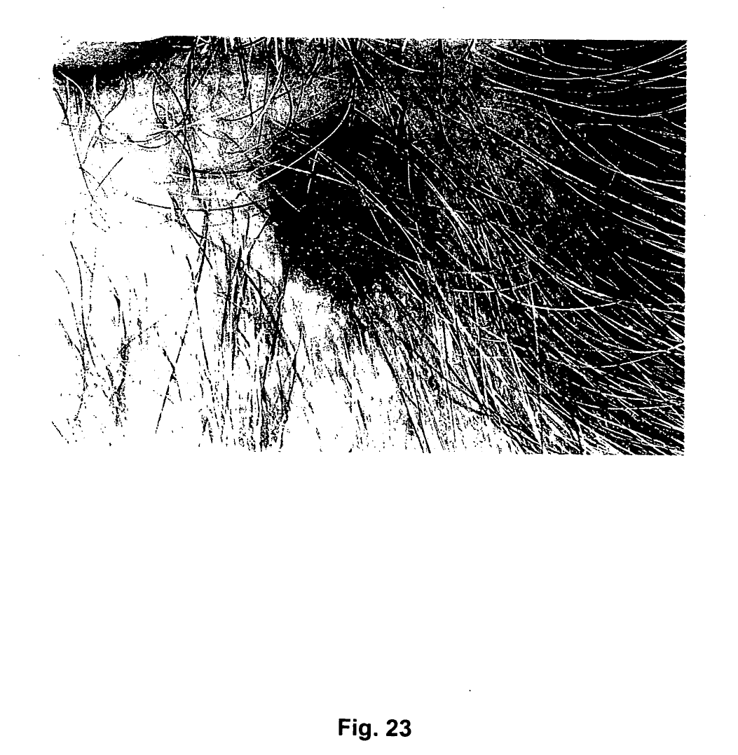

FIG. 23 shows the lesion four hours after treatment. The lesion site showed additional thrombosis and necrosis (shown as darkening), whereas the surrounding normal tissue had begun to improve in appearance.

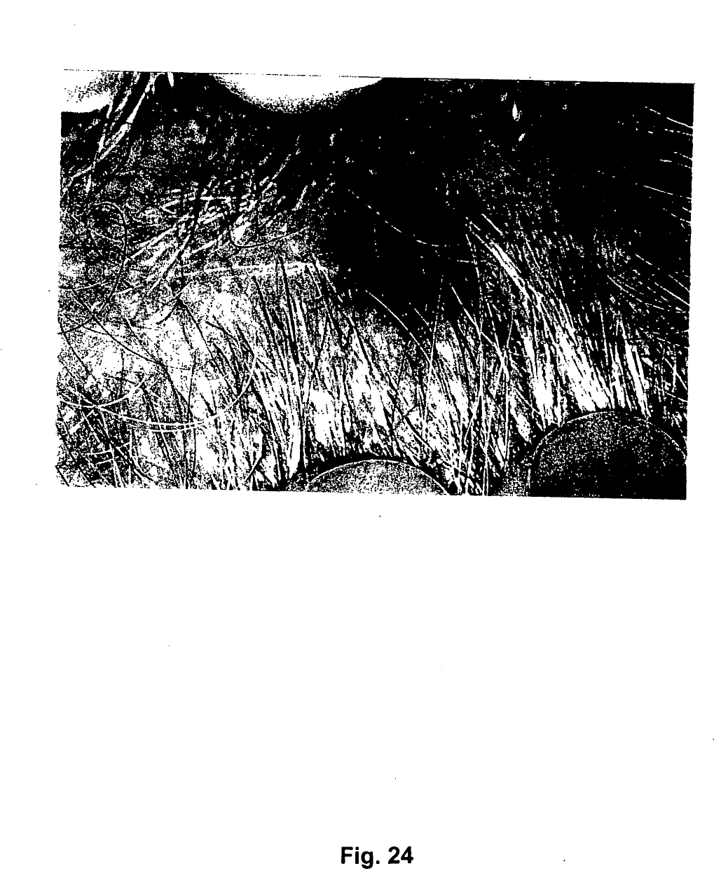

FIG. 24 shows the lesion twenty-four hours after treatment. Eschar formation had begun.

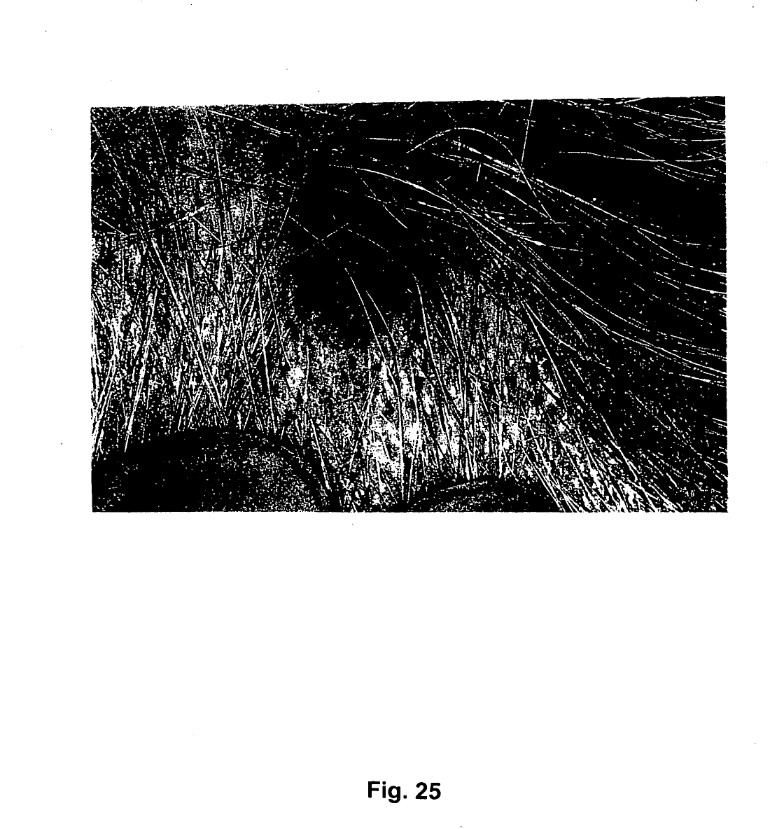

FIG. 25 shows the lesion forty-eight hours after treatment. The lesion had become completely necrotic and thrombosis was extensive. The lesion sloughed off approximately five days later.

These results confirmed the fact that normal cells have protection from the oxidizing properties of singlet oxygen. In contrast, most of the published data relating to reactive oxygen species, reactive oxygen intermediates or reactive oxygen metabolites have indicated that peroxides, hypochlorites and singlet oxygen are all capable of being detrimental and damaging to cellular components, such as lipids, proteins and nucleic acids (see, e.g., Nagano, T., Chemical and Biochemical Studies on Reactivities, Formations and Toxicities of Reactive Oxygen Species, Yakugaku Zasshi 111(2): 103-119 (1991)). These published data have been mainly obtained by in vitro experimentation and have not taken into account the anti-oxidation enzymatic systems and the in vivo anti-oxidant agents. Consequently, researchers have kept away from singlet oxygen in vivo studies.

Therefore, this Example shows the results, which are against the trend of the prevalent approaches, thereby emphasizing the uniqueness of the present invention. It is also noted that even though singlet oxygen has a short lifetime, its beneficial effects continue for months following its generation at the site of application or injection. Without wishing to be bound by any particular theory, it is believed that cellular signaling, which impacts gene expression, DNA synthesis, and/or cellular proliferation, is being altered by the presence of the singlet oxygen of the invention.

### Example 2

### Treatment of Keratosis II

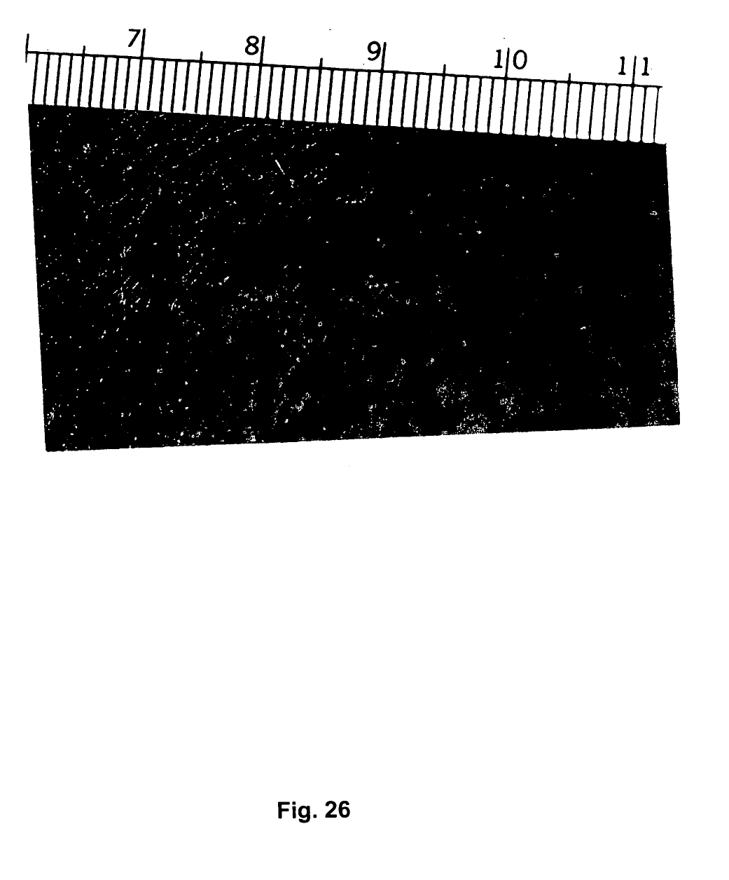

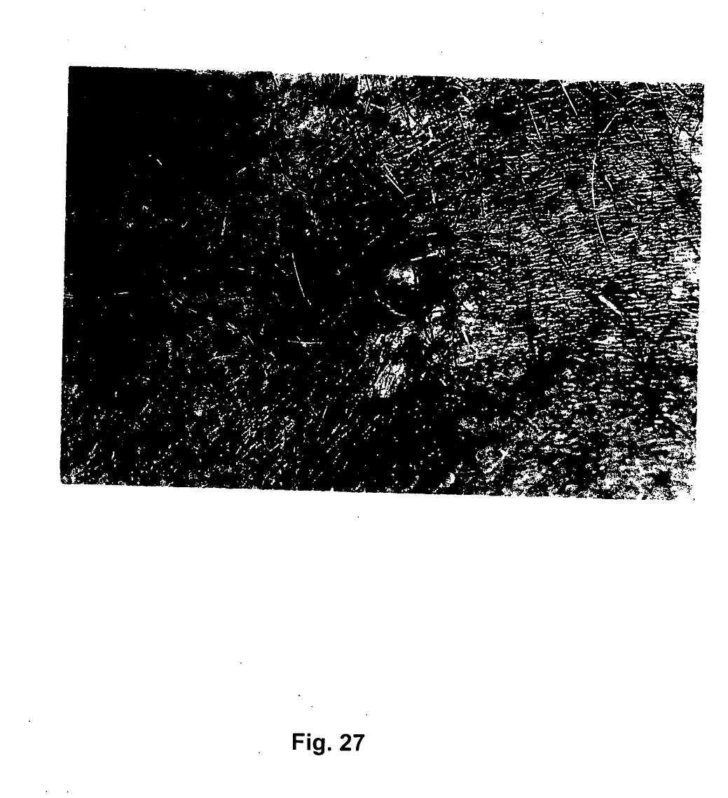

Three skin keratosis lesions from a 66-year old white male were chosen as target sites for treatment: (1) a singular, non-pigmented, dermal nevus measuring 0.7 cm in diameter and 0.2 cm in height located in a right upper scapular area (lesion A); (2) a pedunculated pigmented nevus measuring 0.4 cm in diameter and 0.3 cm in height located above a left supra clavicular area (lesion B); and (3) a pigmented senile keratosis measuring 0.7 cm in diameter located in a right upper abdominal quadrant (lesion C). Photos of each lesion, prior to treatment, are shown in FIGS. 26 (lesion A), 34 (lesion B), and 42 (lesion C), respectively.

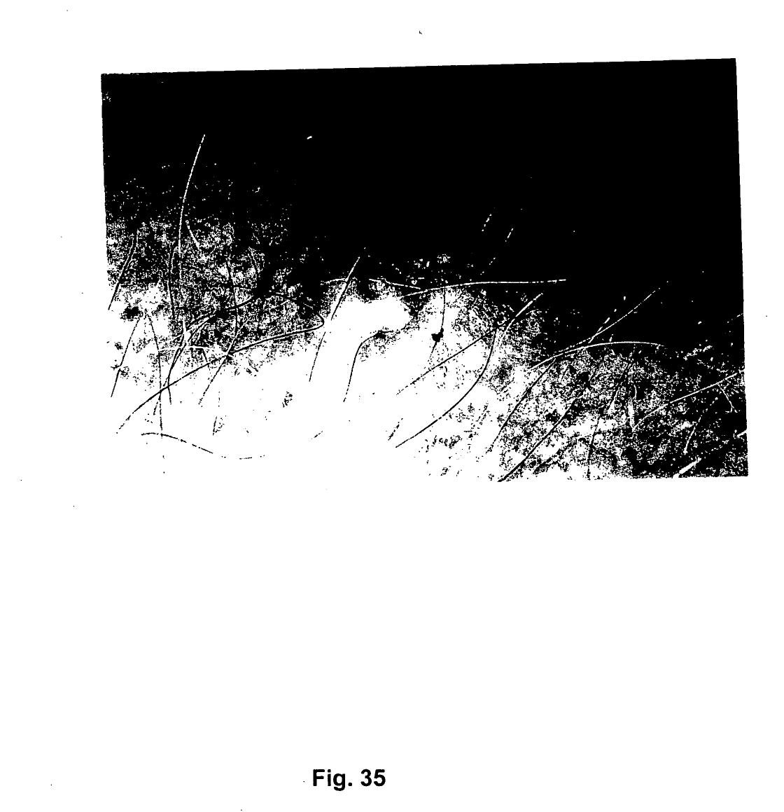

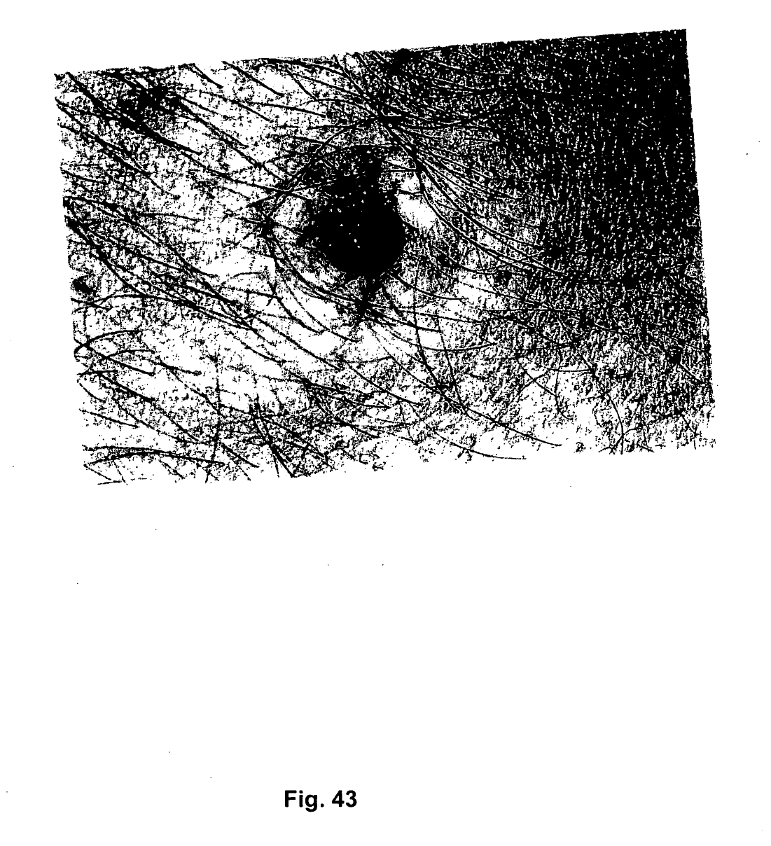

Using a 25-gauge needle attached to a 1 ml syringe, the lesion A was injected at the inferior border, and the needle tip was advanced to the center of the lesion, while infiltrating 0.22 ml of a 6% solution of sodium hypochlorite. This process was immediately followed with 0.44 ml of a 3% solution of hydrogen peroxide. This injection procedure was repeated for other lesions. The lesion B was injected with 0.2 ml of a 6% solution of sodium hypochlorite, immediately followed with 0.2 ml of a 3% solution of hydrogen peroxide. With regard to the lesion C, 0.1 ml of a 6% solution of sodium hypochlorite, immediately followed with 0.2 ml of a 3% solution of hydrogen peroxide were injected. All lesions A, B, and C showed blanching and vasoconstriction of the lesions immediately following the injections of the reactants. FIGS. 27, 35, and 43 show the lesions A, B, and C, respectively, immediately after injection with sodium hypochlorite and hydrogen peroxide.

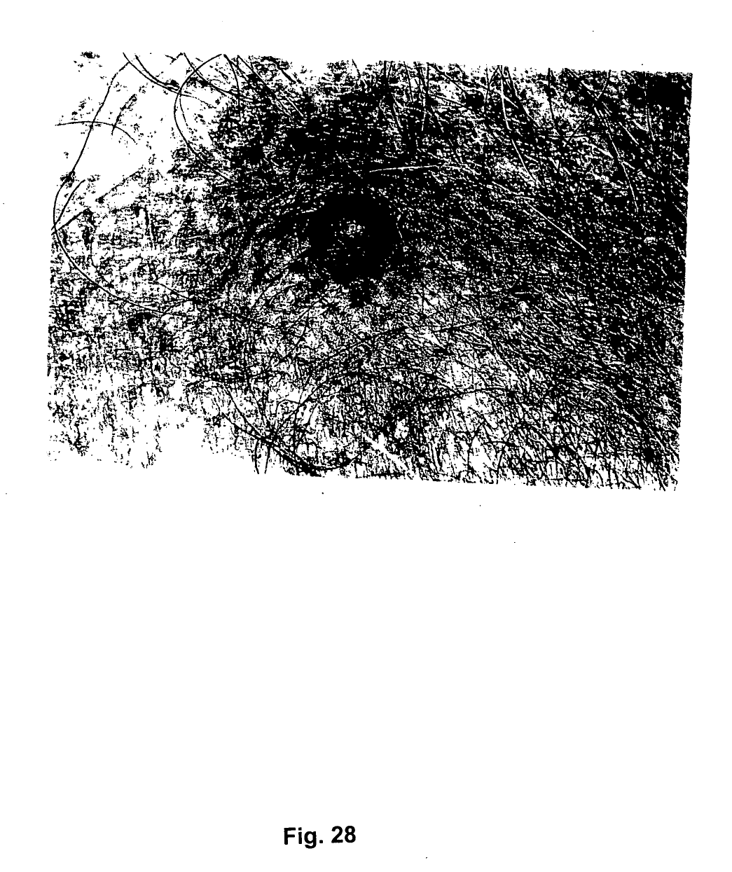

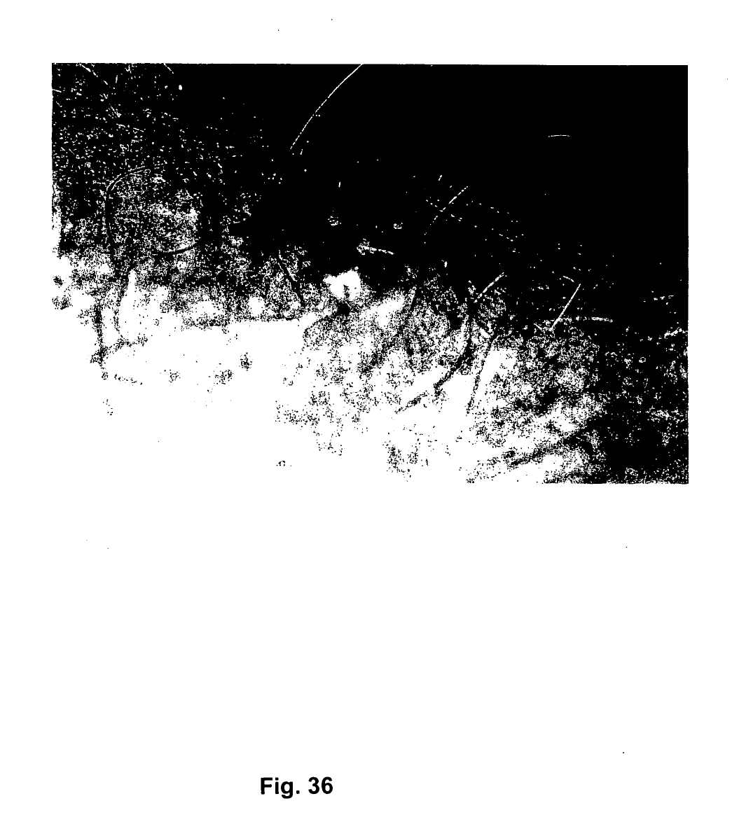

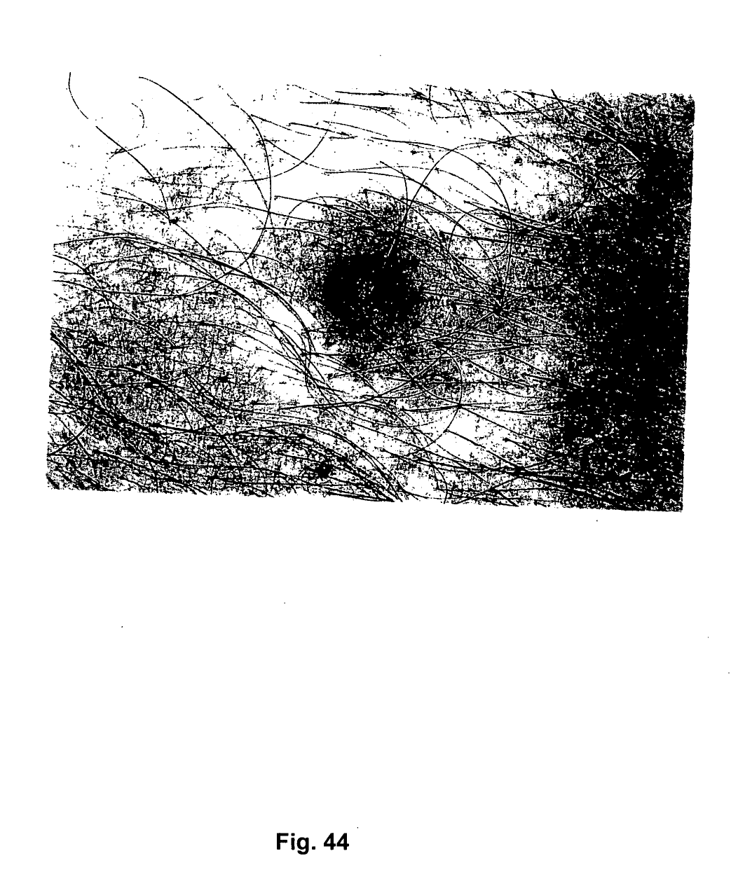

FIGS. 28, 36, and 44 show the respective lesion sites A, B, and C three minutes after the injections. The blanchings in the areas surrounding the injections indicated extreme vasoconstriction. The blanched tissues were normal tissues surrounding the lesions. In particular, the lesion C produced a blanched area exceeding 3 cm in diameter within 3 minutes following the injection as shown in FIG. 44. Bleeding from all lesion sites A, B, and C was minimal. Partial thrombosis occurred within 3 minutes following the injections as shown FIG. 28.

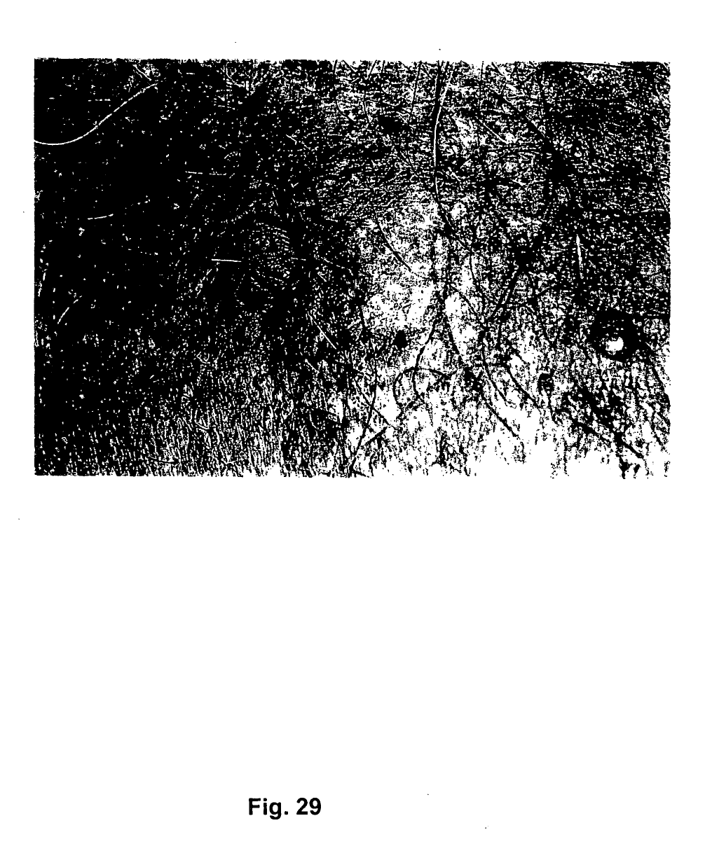

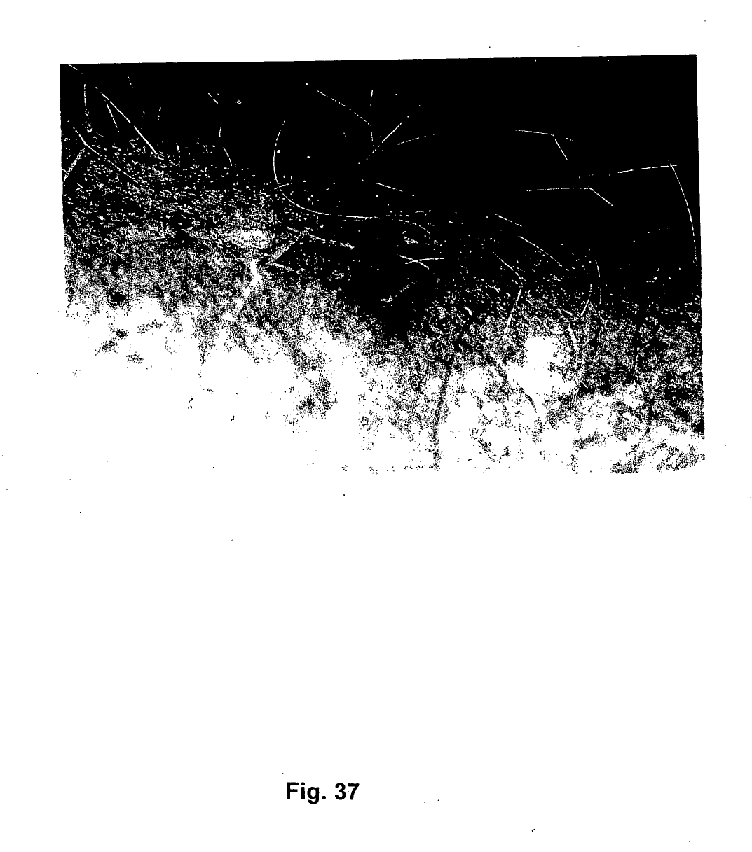

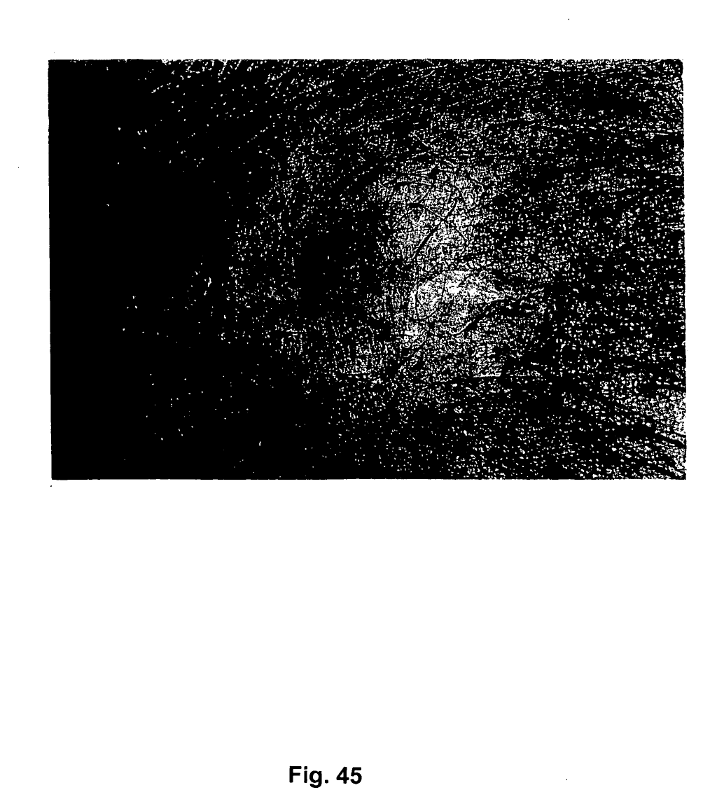

FIGS. 29, 37, and 45 show the respective lesion sites A, B, and C 24 hours after the treatment. As shown in FIGS. 29, 37, and 45, complete lesion thromboses occurred within 24 hours following the injections. In particular, FIG. 45 showed considerable contusion (bruising) of the surrounding area. Different reactions from each lesion suggest differences in the biochemical structure of the skin and dermis in different parts of the body.

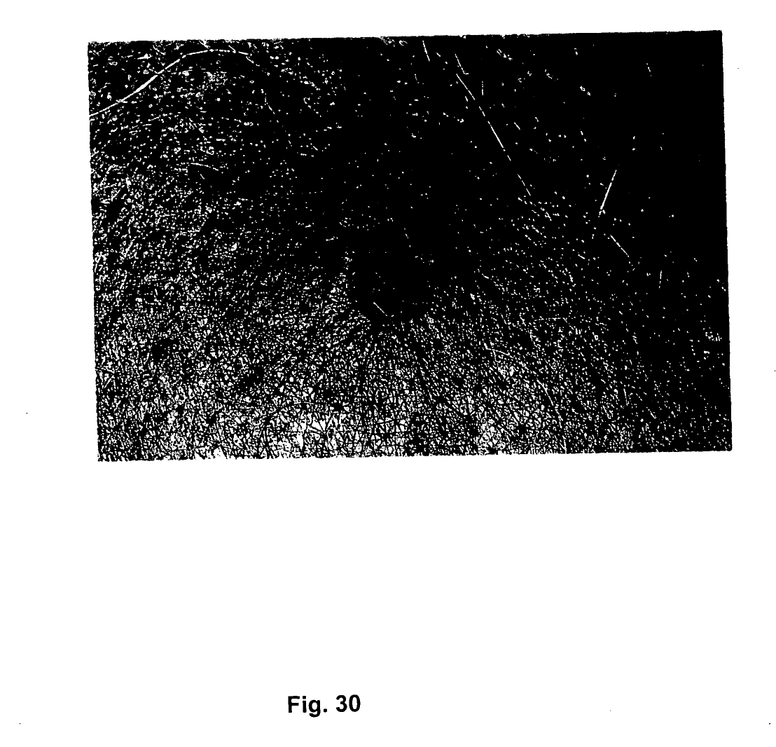

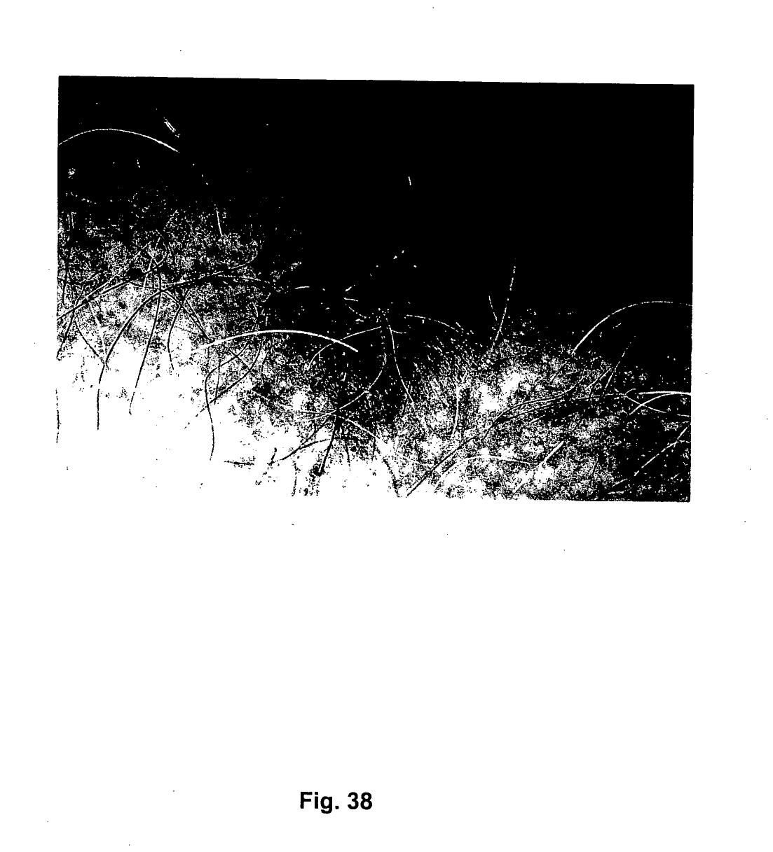

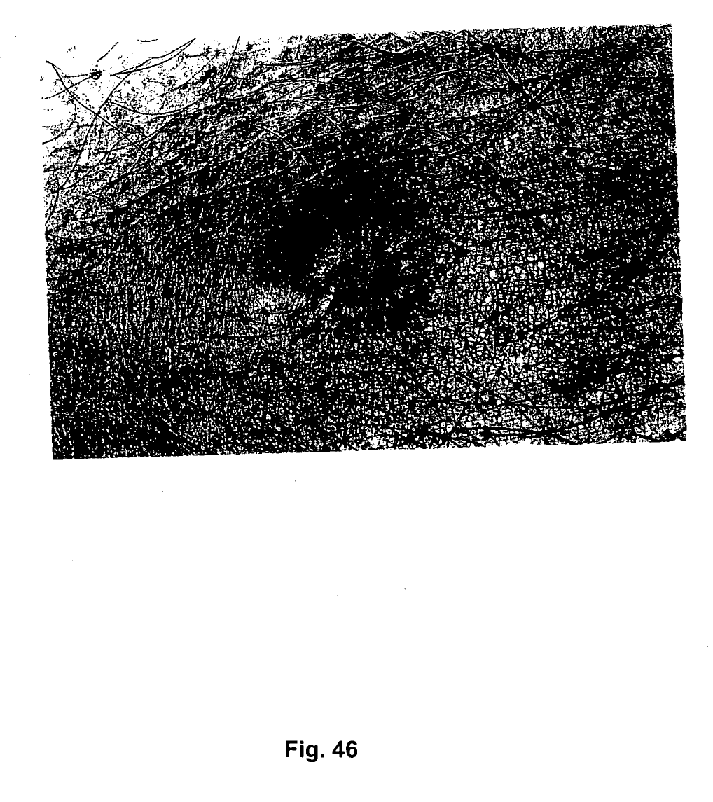

As shown in FIGS. 30, 38, and 46, all lesions A, B, and C indicated signs of apoptosis (cell death) and sequelae of vascular thrombosis (clotting) by day eight. All lesions A, B, and C were surrounded by areas of erythema (redness).

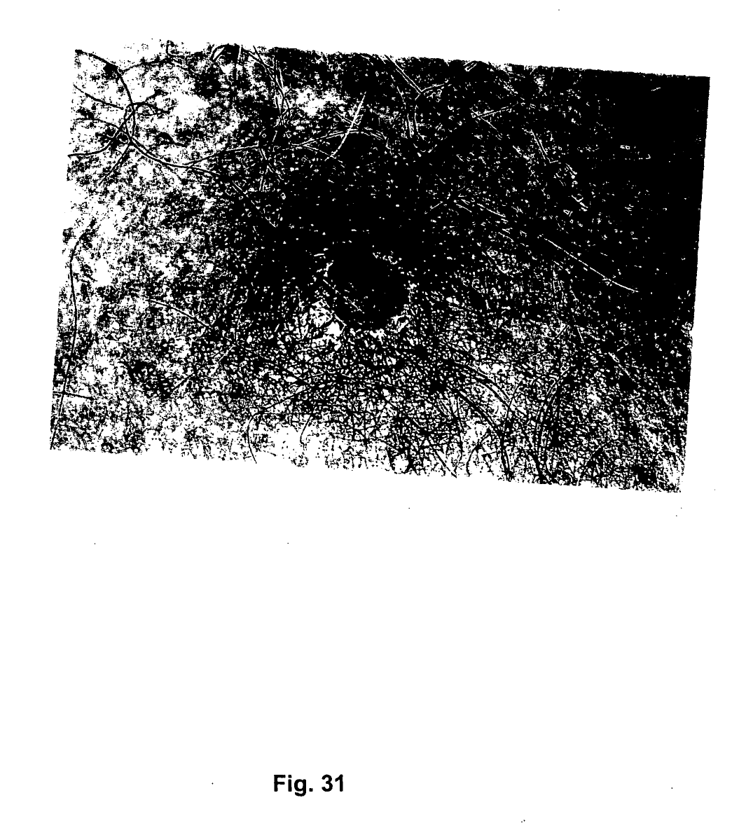

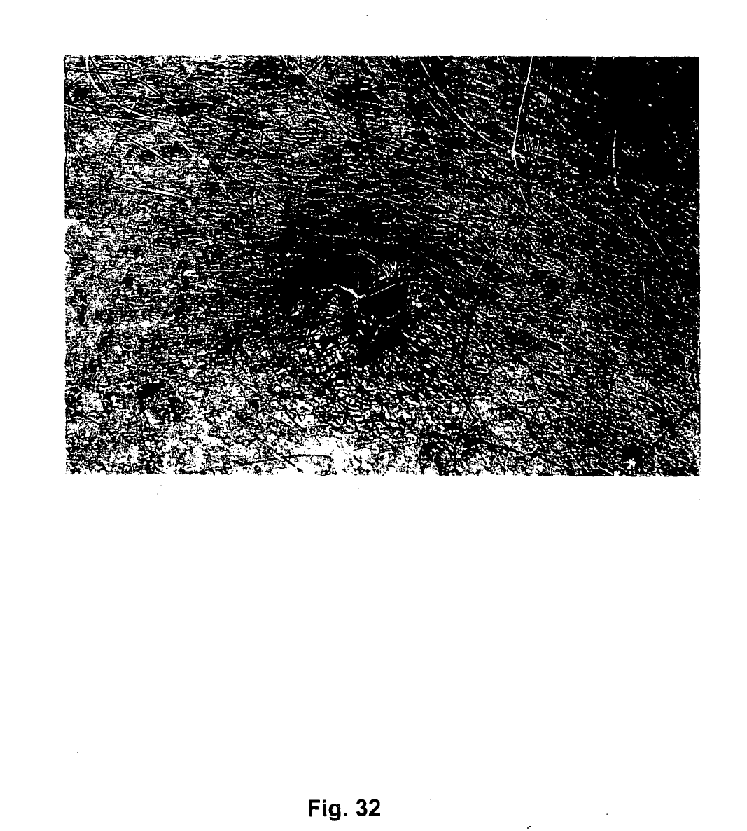

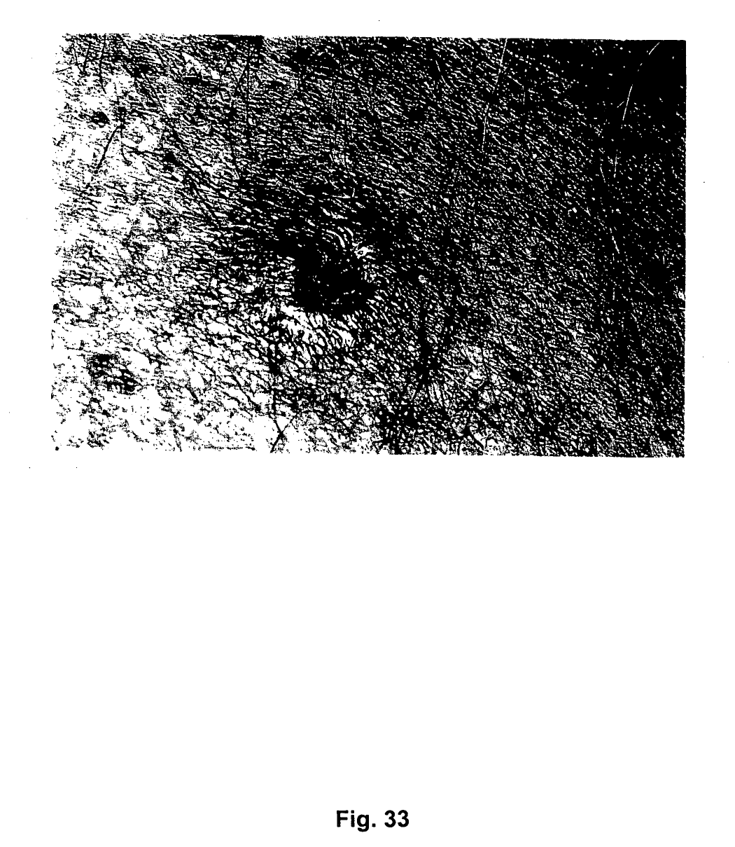

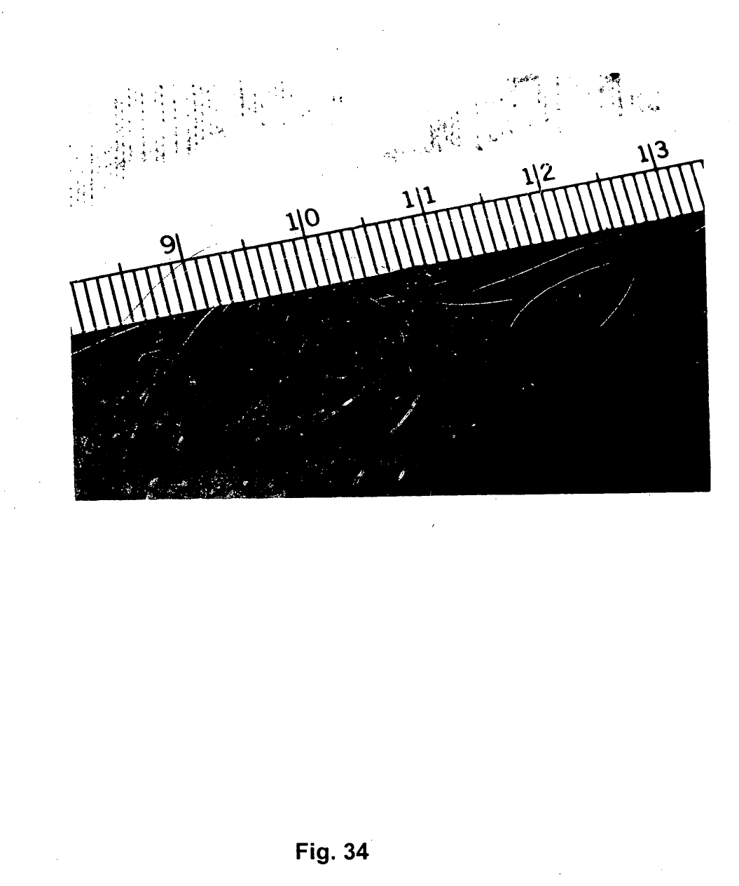

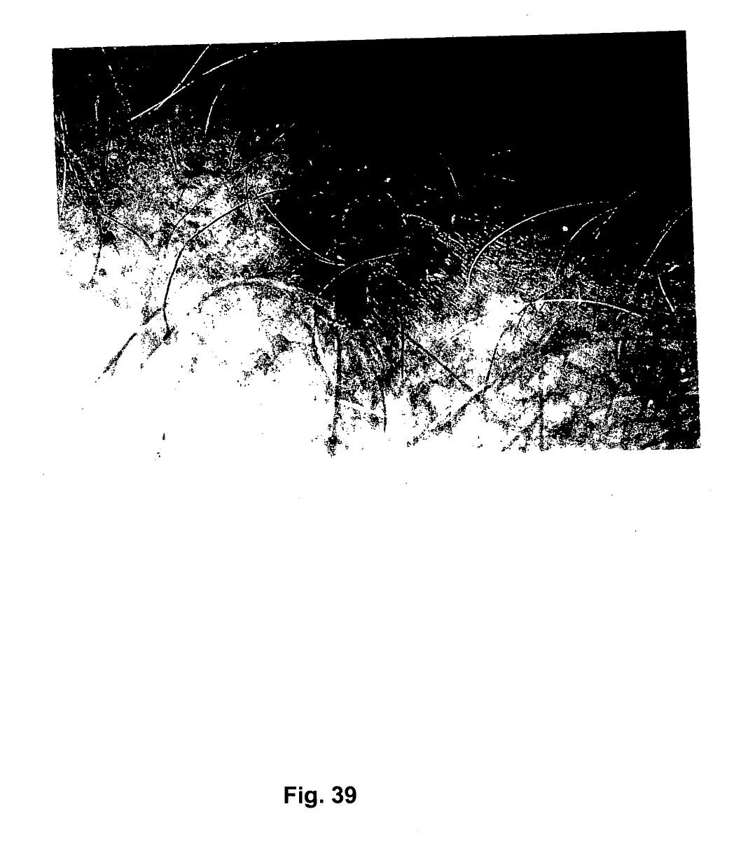

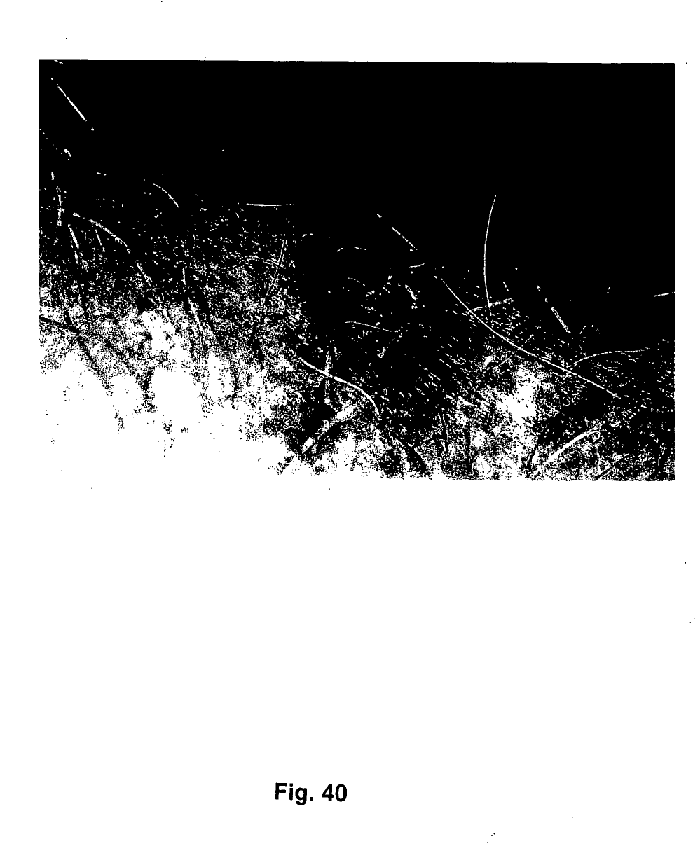

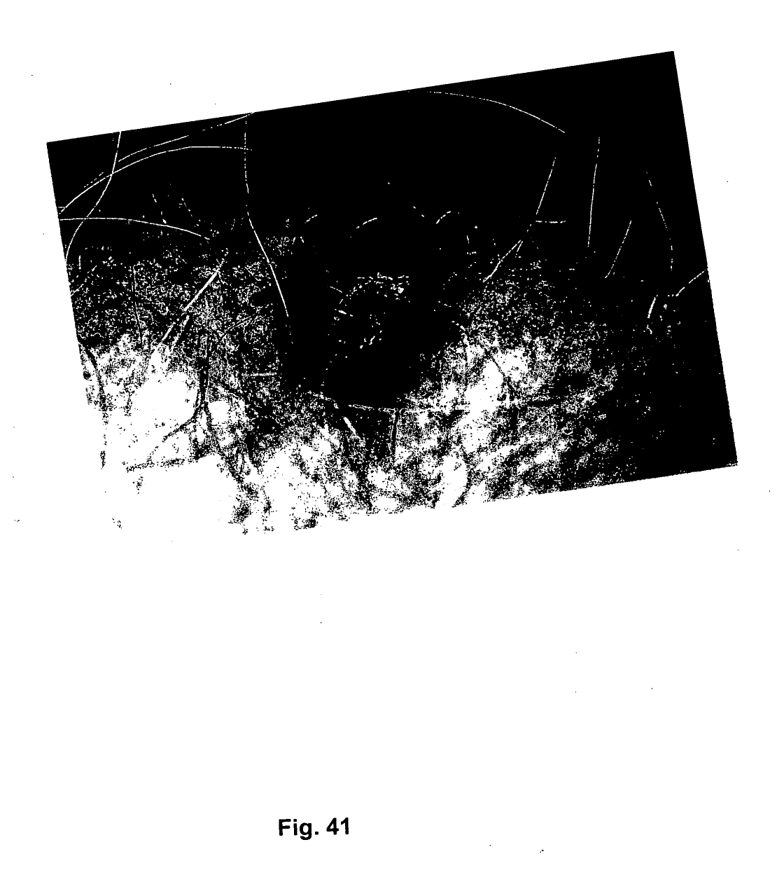

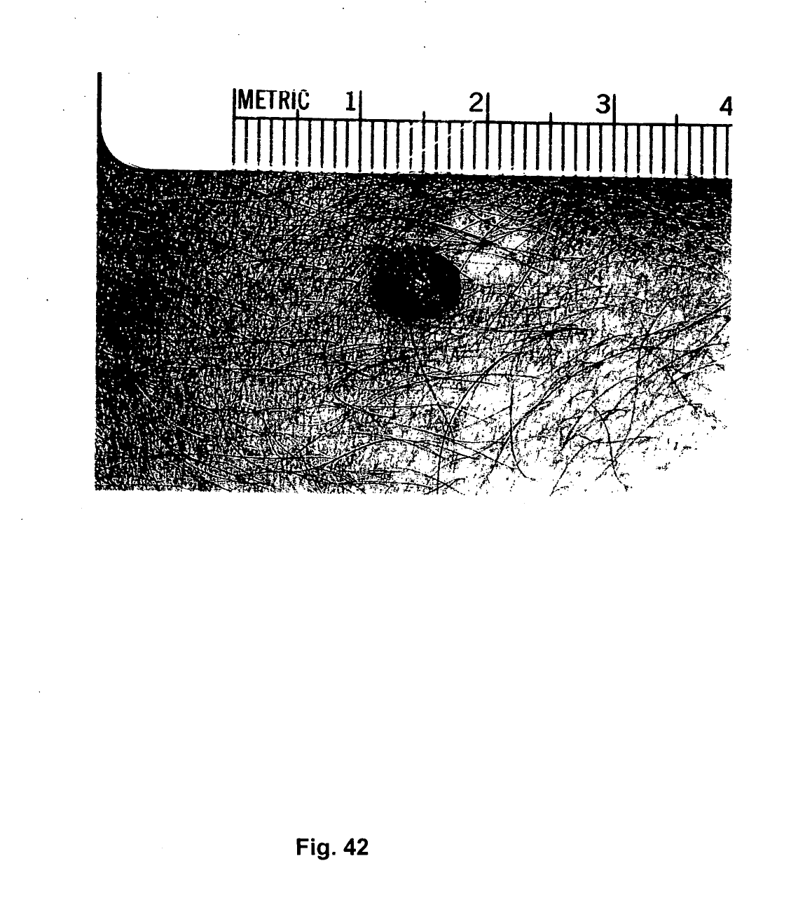

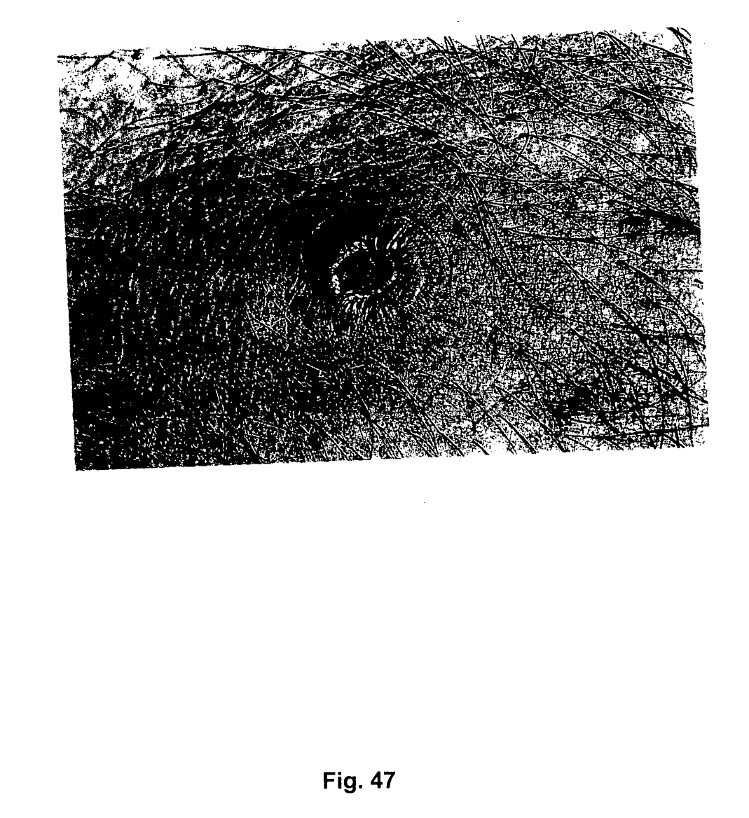

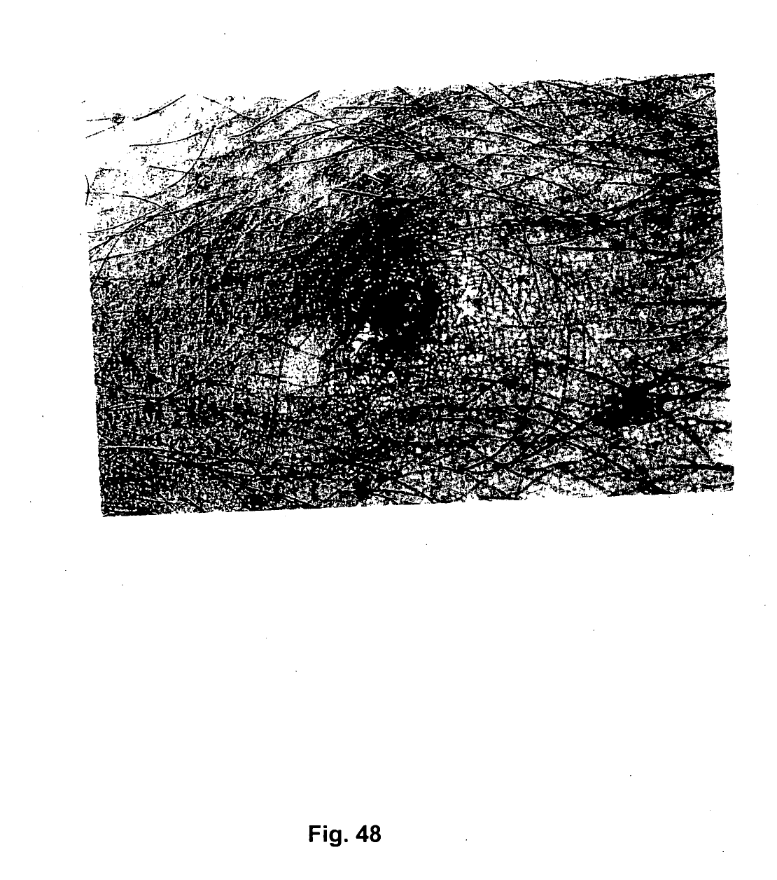

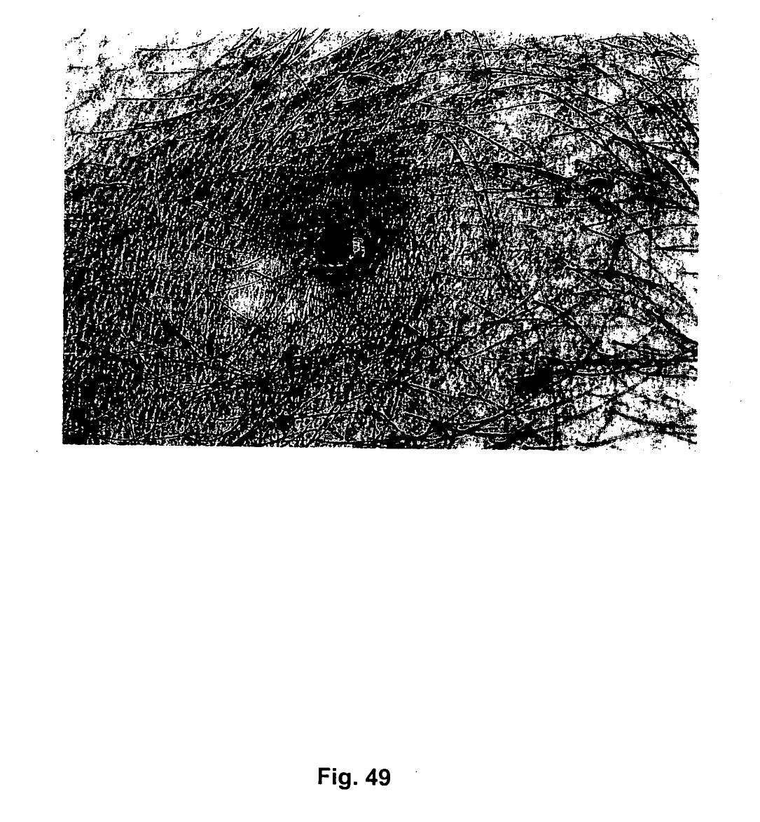

FIGS. 31, 39, and 47 show the respective lesion sites A, B, and C thirteen days after the treatment. Corresponding photographs of the lesions A, B, and C twenty six days after the treatment are shown in FIGS. 32, 40, and 48, respectively. Finally, FIGS. 33, 41, and 49 show the corresponding lesion sites A, B, and C thirty three days after the treatment. During this period, various stages of sloughing (shedding) and necrotic (dead) lesions with subsequent dermal healing were demonstrated. All lesion sites were contracting by the thirty third day following the injections. These lesion sites also demonstrated pitting (sunken centers), as healing progressed. These phenomena indicated that the initial lesions had extended (with roots) deeply into the dermis and subdermis. The patient described only mild discomfort during any phase of the treatment.

It was observed that singlet oxygen produced from the injected reactants selectively killed and removed only abnormal skin cells. This observation suggests that normal cells possess appropriate protective enzymatic systems to prevent them from being permanently harmed. On the other hand, no corresponding protective mechanisms were observed with regard to cancerous or mutated cells based on the results of singlet oxygen treatment. Therefore, these results demonstrated that abnormal human skin lesions can effectively be treated with the singlet oxygen generated by the injected reactants.

### Example 3

### Treatment of Sclerotic Plaque I

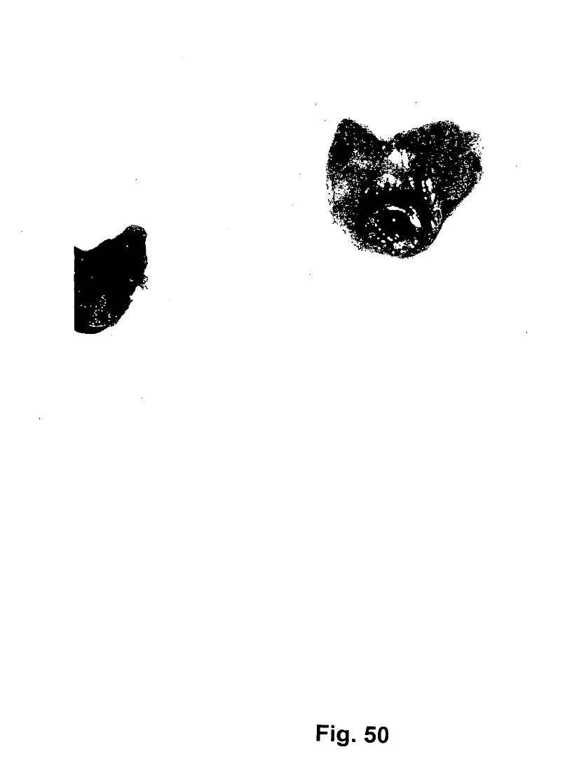

A coronary artery blocked with sclerotic plaque, taken from a human cadaver, was chosen as a target site for treatment. The coronary artery was grayish pink in color and suspended in a formaldehyde preservative. It was surrounded with fatty tissue of an irregular shape, which measured approximately 1 cm in its widest dimension. It measured 0.6 cm in length and 0.5 cm diameter. The lumen of the vessel was completed occluded with sclerotic plaque. A photograph of the human coronary artery with the sclerotic plaque, prior to treatment, is shown in FIG. 50.

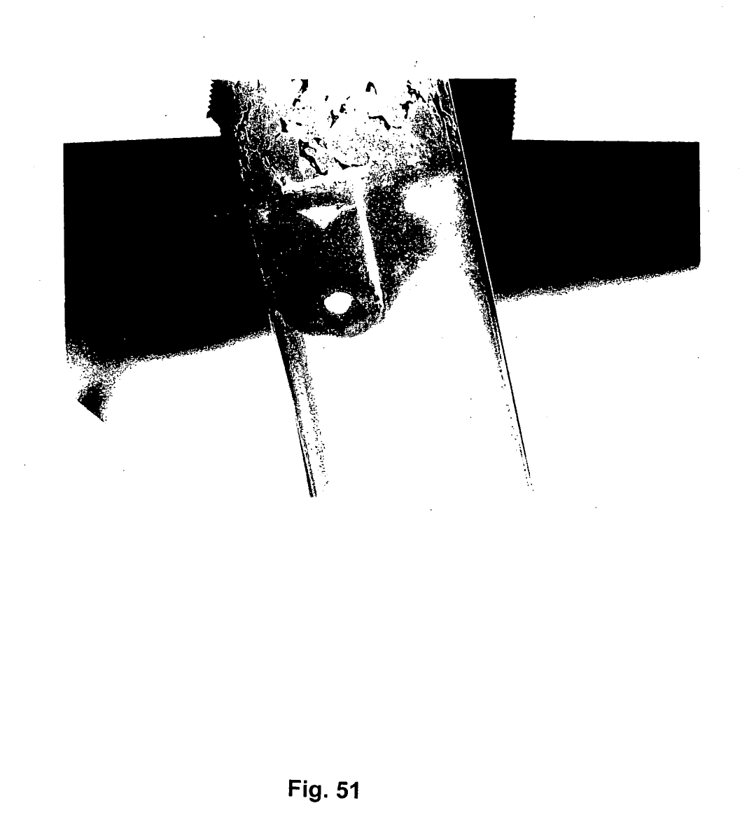

The cross section of coronary artery was placed in a test tube containing 2 ml of 6% of sodium hypochlorite and was shaken for one minute. There was minimal solubilization of the surrounding fatty tissue of the vessel. Next, 2 ml of 3% hydrogen peroxide was added to the test tube to generate singlet oxygen. This hydrogen peroxide had to be added in 1 ml aliquots to avoid extreme reaction and bubbling. This preparation was then again shaken. Within three minutes the occlusion in the artery had been removed and solubilized. Another 2 ml of hydrogen peroxide was added until no further reaction with the sodium hypochlorite occurred. Minimal amounts of fatty tissue were further solubilized. In sum, the treatment with sodium hypochlorite solution and hydrogen peroxide solution cleared and opened the occlusion of the coronary artery within several minutes. FIG. 51 shows the human coronary artery after the treatment.

### Example 4

### Treatment of Sclerotic Plaque II

The following tests were performed to corroborate that the singlet oxygen produced by the hydrogen peroxide and the sodium hypochlorite had actually cleared and opened the human coronary artery blocked with the sclerotic plaque.

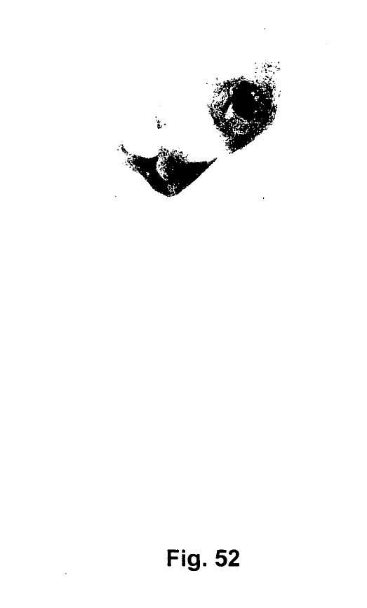

Like Example 3, a coronary artery blocked with sclerotic plaque, taken from a human cadaver, was prepared as a target site for treatment. The coronary artery was grayish pink in color and suspended in a formaldehyde preservative. It was surrounded with fatty tissue of an irregular shape, which measured 1.1 cm in its widest dimension. It measured approximately 0.6 cm in length and 0.5 cm in diameter. The lumen of the vessel was completed occluded with sclerotic plaque. A photograph of the human coronary artery with the sclerotic plaque, prior to treatment, is shown in FIG. 52.

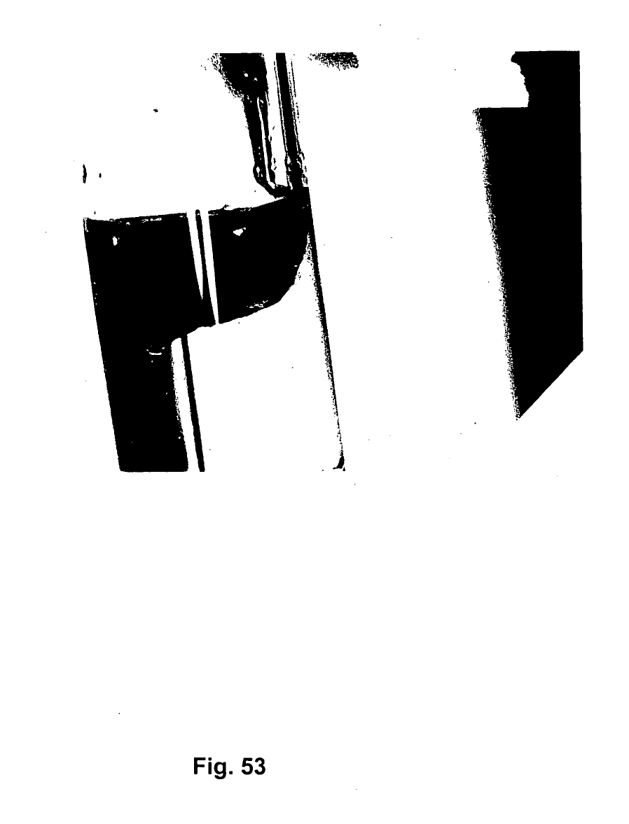

The human coronary artery with the plaque was first treated with 2 ml of 6% solution of sodium hypochlorite alone. As shown in FIG. 53, no significant changes occurred to the plaque.

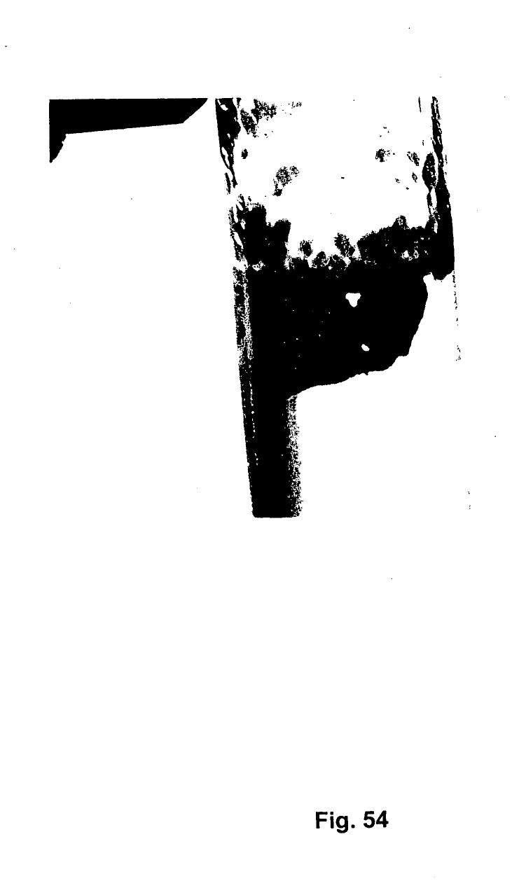

Immediately after the treatment with the sodium hypochlorite, 2 ml of 3% solution of hydrogen peroxide was added to the 2 ml of 6% solution of sodium hypochlorite containing the human coronary artery with the plaque. As shown in FIG. 54, substantial changes were observed including opening of the artery and dissolution of the sclerotic plaque, as did in Example 3. These results clearly show that singlet oxygen produced from hydrogen peroxide and sodium hypochlorite cleared and opened the human artery blocked with the plaque. This Example demonstrates that singlet oxygen was produced when a sodium hypochlorite solution and a hydrogen peroxide solution are administered to the target site.

### Example 5

### Treatment of Sclerotic Plague In Vivo

Two solutions are prepared: 1) 0.5 M hydrogen peroxide in reverse-osmosis water and 2) 0.5 M sodium hypochlorite in reverse-osmosis water. As illustrated in FIG. 8, a dual lumen catheter with proximal and distal ports is prepared. The two solutions are held in separate IV bags, and delivery conduits connect the IV bags to the dual lumen catheter, as shown in FIG. 8. The catheter is inserted proximal to a sclerotic plaque, and the two reactants (sodium hypochlorite solution and hydrogen peroxide solution) are infused. The sodium hypochlorite solution is delivered from a proximal port, and the hydrogen peroxide solution is delivered from a distal port, as shown in FIG. 8. The reaction between the two reactants takes place to create a constant supply of singlet oxygen at the plaque, thereby dissolving the plaque.

The dissolution of the sclerotic plaque is monitored by measuring the blood flow of the surrounding area after the treatment by standard radiographic or flow measurement techniques. If necessary, treatment is repeated.

### Example 6

### Treatment of Equine Carcinoma I

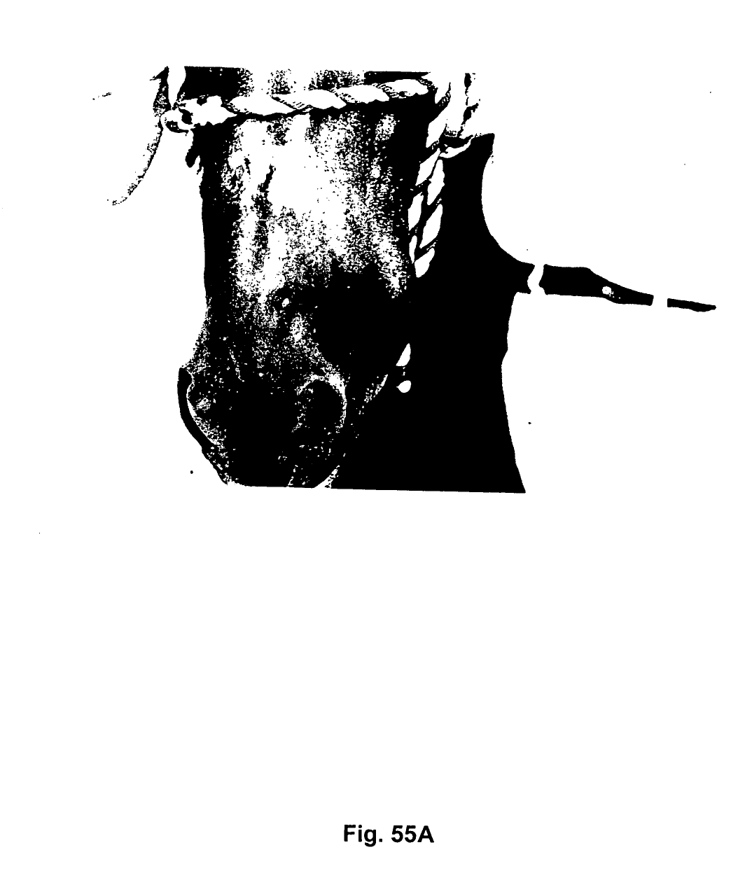

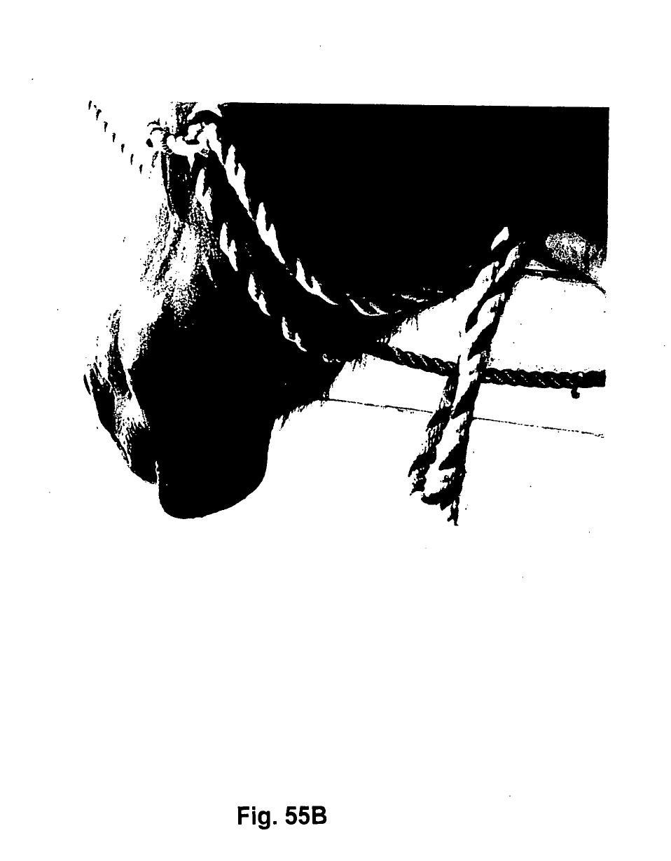

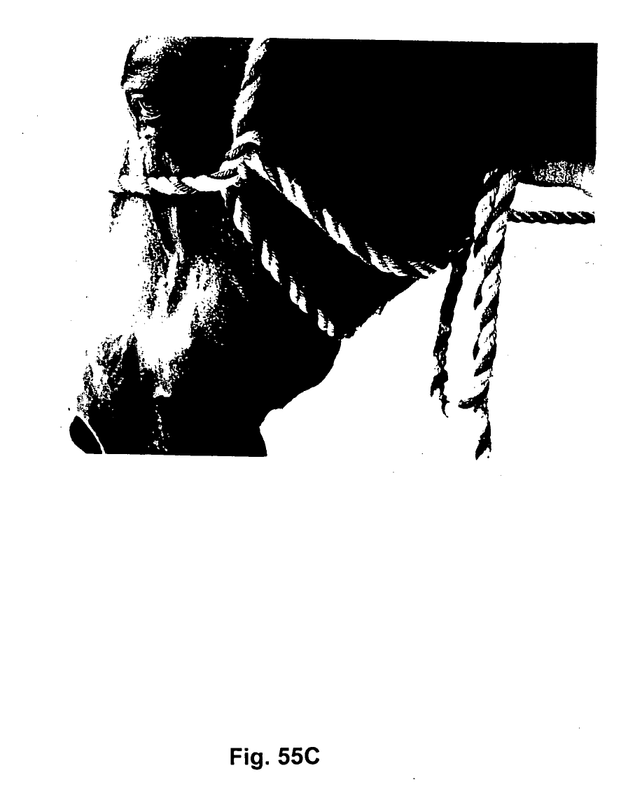

A horse having an equine squamous cell carcinoma was chosen to test the effect of singlet oxygen of the present invention on the morphological change of the carcinoma. Since the horse was in a terminal phase with multiple tumors, the treatment of the carcinoma was not intended to cure the horse. Instead, the study was conducted to evaluate the impact on a single tumor. The estimated height of the horse was 15¾ hands with an estimated weight of 950 lbs. Photographs of the horse having the equine squamous cell carcinoma, taken from different directions and prior to treatment, are shown in FIGS. 55A, 55B, and 55C.

Initial inspection of the lesion selected for evaluation revealed that it was located in the left canine-facial-gingival area and was measured approximately 8 inches by 4 inches. It was elliptical in shape, solid, and firm to palpation. Intra-oral examination showed a large, firm, pink-colored mass located superior to the left upper canine tooth.

The horse was given I.V. tranquilizers consisting of detomidine hydrochloride, butorphanol tartrate, and xylazine. Local anesthesia and nerve blocks were obtained by injecting the lesion circumferentially with 2% solution of xylocaine (2.0 ml of lidocaine hydrochloride, 0.2 ml of sodium chloride, 0.2 ml of potassium phosphate monobasic, 0.2 ml of potassium phosphate dibasic, 0.1 ml of methylparaben, sterile water for injection q.s.).

The tumor lesion was injected with a 21-gauge spinal needle attached to a 12 ml syringe. A 6% solution of sodium hypochlorite was injected first, immediately followed by a 3% solution of hydrogen peroxide. With regard to further injections, 19-gauge needles were used since the movement of the horse made the further injections difficult. Bleeding was minimal from the injection sites and bubbling was produced in some areas as a result of either reaction of the hydrogen peroxide with the sodium hypochlorite or reaction of blood catalase with the hydrogen peroxide. The mass increased in size following the reactant injections.

Total amount of injected reactants was 11 ml of 6% solution of sodium hypochlorite and 12 ml of 3% solution of hydrogen peroxide. The horse tolerated the entire procedure, which took approximately 45 minutes.

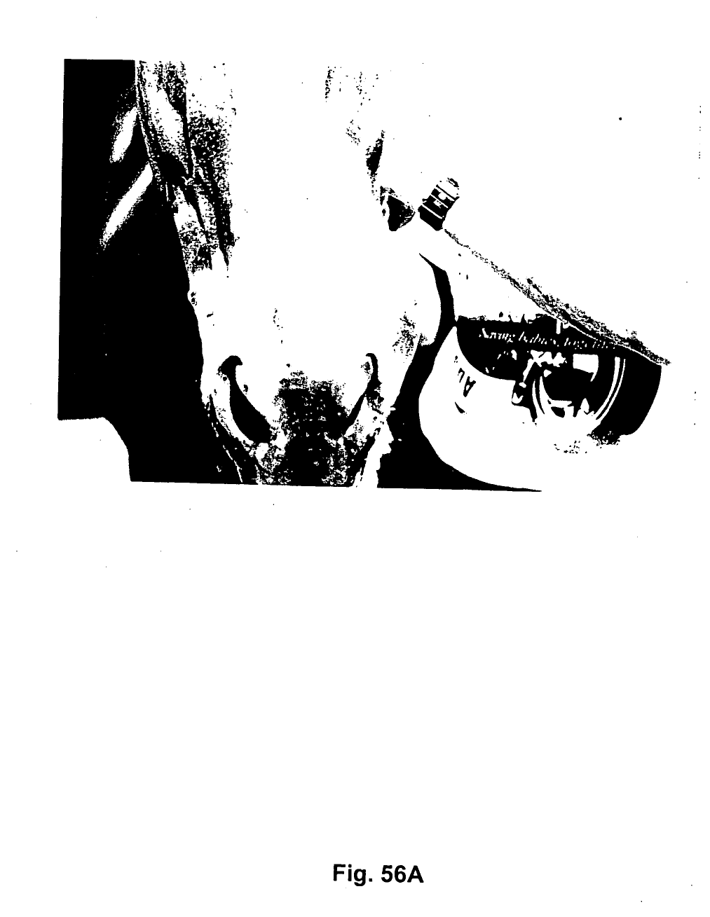

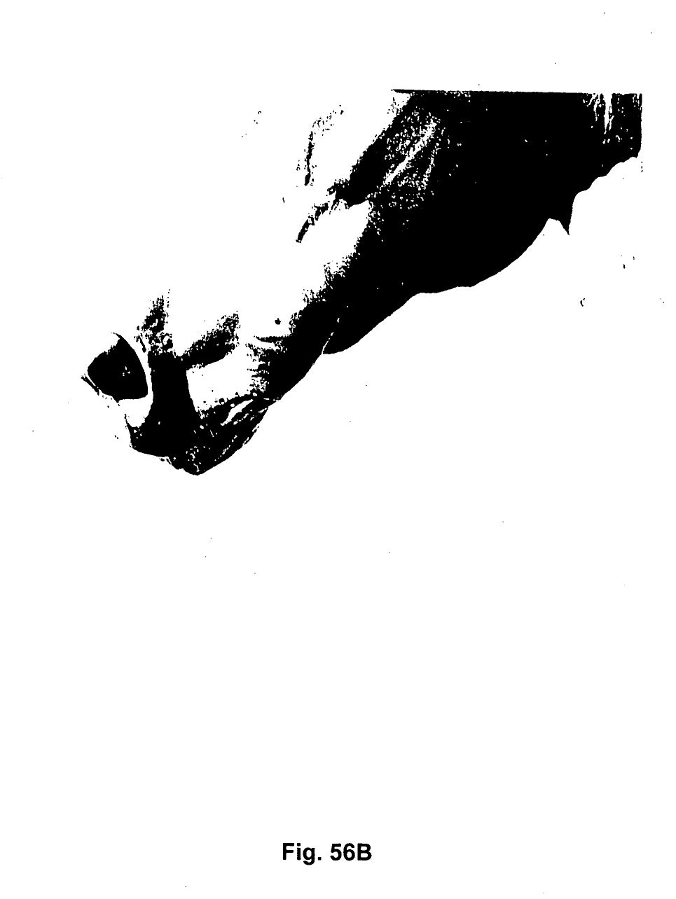

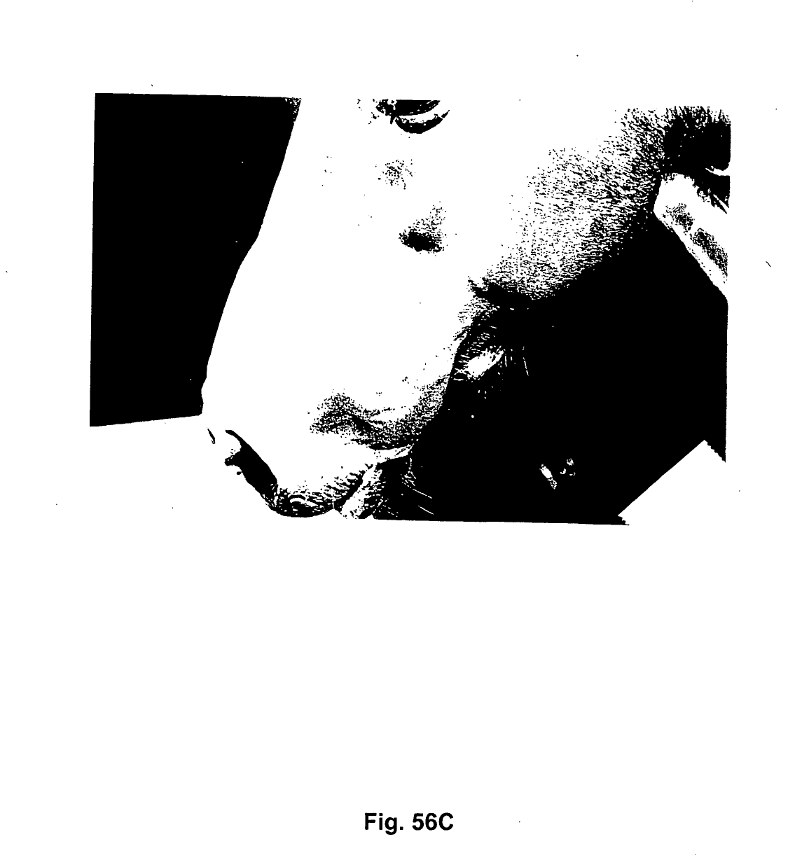

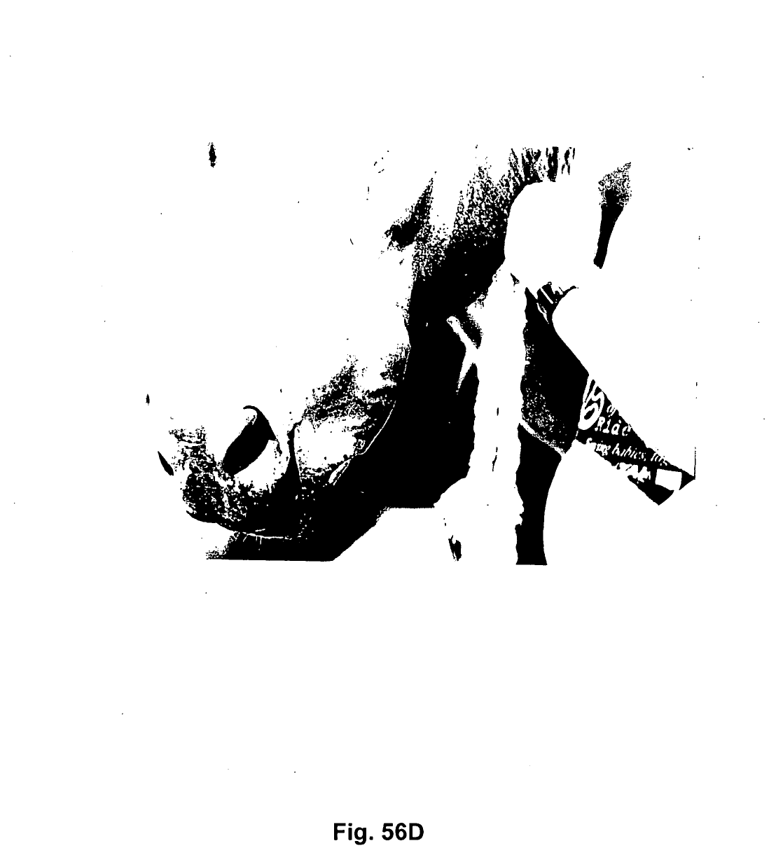

There appeared to be a decrease in the size of the tumor assessed approximately 1 month after the injections, as shown in FIGS. 56A, 56B, 56C and 56D, which are photographs of the horse, taken from different directions and approximately 1 month after the injections.

### Example 7

### Treatment of Equine Carcinoma II

Additional injections were made on the horse of Example 6 under general anesthesia approximately 1 month after the injections of Example 6, in order to treat the selected equine carcinoma in its entirety, to further reduce the size of the tumor, and to control its growth. Again, because the horse was in a terminal phase with multiple tumors, the treatment of the carcinoma was not intended to cure the horse completely.

General anesthesia was obtained utilizing the following: xylazine (100 mg/ml), detomidine HCl (10 mg/ml), and torbugesic (butorphanol tartrate) (10 mg). After obtaining adequate anesthesia, the lesion was cleaned with betadine solution. To expose the large tumor region, the labial areas were retracted. Visual inspection revealed that the appearance had changed from the previously smooth, light pink uniform surface to a surface with dark red healing areas and breaches in the epithelium, representing healing areas of necrosis.

Injections were made with 18-gauge spinal needles attached to 12 ml syringes. First, the needle for 6% solution of sodium hypochlorite was inserted, then the needle for 3% solution of hydrogen peroxide was placed immediately adjacent and parallel to the first needle. Reactants were injected upon withdrawal of the respective syringe and needle. The sodium hypochlorite solution was injected first, followed by the hydrogen peroxide solution. All injections were carefully performed to get even and homogeneous amount of reactants along the needle tracts.

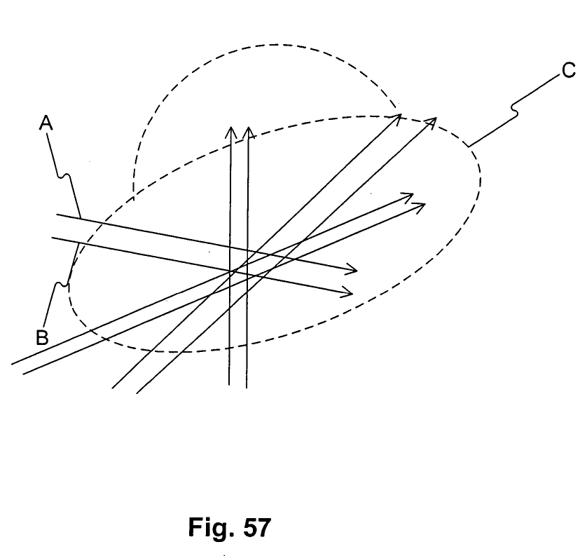

As shown in FIG. 57, four sets of injections (four times for sodium hypochlorite solution A and four times for hydrogen peroxide solution B) were made in four different directions, so that the reactants could reach uniformly the entire parts of the carcinoma C. The border of the carcinoma is indicated by a broken line.

The tumor had possible central and internal necrosis areas, as evidenced by changes in the degree of difficulty of needle insertion. Reactant injection also forced purulent material to the outside of the tumor. These results clearly confirmed that necrotic areas existed inside the large tumor mass. It appeared that pockets of necrosis existed inside the tumor after the injections of Example 6.

Total amount of injected reactants was doubled compared with that of Example 6 (24 ml of 3% solution of hydrogen peroxide and 24 ml of 6% solution of sodium hypochlorite). The horse tolerated the whole injection procedure very well without any incident. Post-injection observation indicated that the horse had no evidence of pain on the second post-injection day, but had some swelling. Progress of treatment was monitored by observing changes in color and reduction in tumor size. Although the treatment with the hydrogen peroxide and the sodium hypochlorite changed the morphological structure of the carcinoma, the horse died after the treatment described in Example 7 due to its terminal state and the plurality of tumors it was affected by.

### Example 8

### Decontamination

This example illustrates how a biological contamination, such as anthrax, is decontaminated using the present invention.

A backpack apparatus, such as that illustrated in FIG. 1A, is prepared. Two gallons of a 1 molar solution of hydrogen peroxide in water is prepared and placed in one compartment of the canister. Two gallons of 1 molar sodium hypochlorite is prepared and placed in the second compartment of the backpack canister. Both solutions are made 0.1 molar with respect to sodium dodecyl sulfate, a surfactant. The lids are screwed in place, and the compartments pressurized.

Decontamination personnel are appropriately suited, for dealing with both anthrax and the potent oxidizing agent of singlet oxygen, and the backpack is put on. A target site for decontamination, which has been tested positive for anthrax exposure, or is believed to likely be contaminated with anthrax, is sprayed with the mixture from a distance of at least 10 feet. The reactants combine to produce singlet oxygen at the target site, oxidizing any pathogen present in the area. The surfactant helps lyse any pathogenic cell and improve pathogen destruction. The mixture is left for approximately 5 minutes to decontaminate the target area.

After the reaction is complete, which is essentially immediately after application, any residue may be removed using water.

### Example 9

### Routine Disinfection

This example demonstrates how the invention is applied in a routine manner for disinfection.

The spray bottle, shown generally in FIG. 2A, is prepared. In one compartment is placed a solution of 1 molar hydrogen peroxide, and in the other compartment is placed a solution of 1 molar sodium hypochlorite. Other components in the composition may include detergents, scents, coloring agents, alcohols, etc.

The spray trigger is actuated, resulting in a sequential spray of the hydrogen peroxide solution followed by the hypochlorite solution. Upon mixing of the two solutions at the target site, singlet oxygen is produced, and a powerful oxidization effect results. The other components of the solution enhance the cleansing properties. The residue is then wiped up with water.

### Example 10

### Topical Antiseptic

This example demonstrates how the invention is used for topical cleansing of human skin prior to a medical treatment.

A disposable topical application bottle is prepared as shown in FIG. 3. The bottle is sized to be used in one hand and constructed from flexible plastic material. In one compartment, a 0.3 molar solution of hydrogen peroxide in reverse-osmosis water is prepared and in the other compartment, a 0.3 molar solution of sodium hypochlorite in reverse-osmosis water is prepared. The bottle is assembled with a track and a sealing tape to be removed prior to use.

When the bottle is to be used, the sealing tape is removed and the absorbent pad slid into the track. The absorbent pad is rubbed on the inner arm, where blood is to be drawn, while squeezing the bottle. The reactants simultaneously enter the pad, reacting to form singlet oxygen, which then cleanses the skin of unwanted pathogens. Several strokes are sufficient to render the skin safe for immediate injection.

### Example 11

### Wart Treatment

This example demonstrates how the invention may be used to treat a topical lesion, such as a wart.

Two solutions are prepared: 1) 0.5 M hydrogen peroxide in reverse-osmosis water (solution 1), and 2) 0.5 M sodium hypochlorite in reverse-osmosis water (solution 2). A topical anesthetic is applied to the wart to be treated. A volume of 0.05 ml of solution 2 is drawn into a syringe equipped with a fine gauge beveled tip hypodermic needle, and injected into the center of the 3-mm dermal wart until dermal blanching occurs. The syringe and needle are flushed with reverse-osmosis water, and the process is repeated with solution 1, taking care to inject solution 1 into precisely the same area as solution 2. It is advantageous to inject or apply the hypochlorite solution first, because peroxide is immediately broken down by catalase in the body as soon as treatment begins.

Progress of the treatment is monitored by observing changes in color to the wart. Necrosis of the wart tissue is shown by changes in color to dark brown followed by black. After the color change the wart tissue sloughs off within a matter of days, and with minimal scarring.

### Example 12

### Tumor Treatment I

Two solutions are prepared: 1) 0.5 M hydrogen peroxide in reverse-osmosis water, and 2) 0.5 M sodium hypochlorite in reverse-osmosis water. One milliliter of each of the solutions is poured into two separate syringes, as illustrated in FIG. 7. The conduits and catheter are attached, as illustrated in FIG. 7. Air is purged from the system.

A needle housing for the catheter is used to guide the catheter to its target site, and the needle housing is withdrawn, leaving the catheter in place. A total volume of 1 ml (0.5 ml of each) is injected into a 1-cm diameter tumor. The catheter is withdrawn after delivery of the reactants, and the site is bandaged. In an alternative, an injecting catheter may be used to both inject and deliver the components.

Progress is monitored by X-ray of the tumor over the following weeks, with progress shown by reduction in tumor size. If necessary, treatment is repeated.

### Example 13

### Tumor Treatment II

The following procedures were performed to determine whether chemical generation of singlet oxygen in situ would inhibit the growth of, or kill, implanted human tumors in nude mice. The implanted tumor was allowed to grow to a defined size and then a direct injection of hydrogen peroxide and sodium hypochlorite was made into the tumor mass.

Female 5 to 6 week-old Cr1: NU/NU-NUBR mice weighing 20-25 grams were used for subject animals, which were athymic (nude). Human squamous carcinoma cells (SCC-15) were injected into the mice. The mice were observed daily and when tumors grew to a volume of approximately 0.2 cm³, the anti-tumor treatment was initiated. Mice were anesthetized prior to intratumor injection with hydrogen peroxide and sodium hypochlorite.

The mice were divided into four groups (I, II, III, and IV). Group I acted as a cytotoxicity control group. These mice were injected with no tumor cells, and dermally injected biweekly with either 0.05 ml of 0.18 M sodium hypochlorite followed by 0.05 ml of 0.18 M hydrogen peroxide (low dose group), or 0.05 ml of 0.88 M sodium hypochlorite followed by 0.05 ml of 0.88 M hydrogen peroxide (high dose group) for 16 weeks. High dose group showed bleaching reaction and it lasted about 1 hour. After that period, the high dose group progressively recovered and the reaction diminished. There was no necrosis. Among five mice of the high dose group, three mice died (It is believed that the small mice were unable to cope with the combination of volume and concentration of the reactants). With regard to low dose group, the reaction was much less severe and its duration was shorter. Only one mouse died among five low dose group mice.

Group II acted as a tumor control group. Fifteen mice were injected with tumor cells (SCC-15). Five mice were injected with 2×10⁵ cells and reinjected with 2×10⁶ cells. The remaining ten mice were injected with 2×10⁶ cells. Fourteen out of the fifteen mice developed tumor masses after 10-30 days (mean=18 days). After the tumor reached a defined size (0.2 cm³), 0.1 ml of physiological saline was injected directly into the tumor biweekly. The mice of this group were monitored until the tumor grew to about 1 cm³ or until the mice became very ill. Injection of physiological saline did not make any significant change of tumor cells with respect to the tumor volumes.

Group III receives the same treatment as Group II, except that the implanted tumor is injected with 0.05 ml of 0.18 M sodium hypochlorite followed by 0.05 ml of 0.18 M hydrogen peroxide (low dose treatment group). The tumor volumes and pathologic effects, as well as various other parameters including animal weight and general health parameters, are monitored.

Group IV received the same treatment as Group II, except that the implanted tumor was injected with 0.05 ml of 0.88 M sodium hypochlorite followed by 0.05 ml of 0.88 M hydrogen peroxide (high dose treatment group). Four mice, which developed at least one tumor having a mass of 0.2 cm³, were selected. Specifically, a needle connected to a syringe containing 0.05 ml of 0.88 M sodium hypochlorite was injected into the tumor mass. Immediately after the injection of sodium hypochlorite, the syringe, which had contained sodium hypochlorite, was replaced with a syringe containing 0.05 ml of 0.88 M hydrogen peroxide, without changing the needle, which had been injected into the tumor mass. Effort was made to hold the needle in place. After replacement of the syringe, hydrogen peroxide was injected into the tumor mass.

The results of injection of sodium hypochlorite and hydrogen peroxide into five tumor masses of the four mice are shown in Table 1 (mouse B has two tumor masses 2 and 3).

TABLE 1
Tumor Volume (cm³)

| | No injection | Injection 1 | Injection 2 | Injection 3 | Injection 4 |
|---|---|---|---|---|---|
| Tumor 1 | 0.599 | 0.336 | 0.438 | 0.270 | 0.241 |
| (Mouse A) | | | | | |
| Tumor 2 | 0.598 | Necrosis | Necrosis | Sloughing | 0.077 |
| (Mouse B) | | | | | |
| Tumor 3 | 0.199 | 0.280 | 0.174 | 0.202 | 0.000 |
| (Mouse B) | | | | | |
| Tumor 4 | 0.344 | 0.448 | 0.583 | 0.468 | 0.254 |
| (Mouse C) | | | | | |
| Tumor 5 | 0.476 | 0.050 | 0.064 | 0.038 | 0.091 |
| (Mouse D) | | | | | |
| Mean | 0.433 | 0.278 | 0.315 | 0.245 | 0.133 |
| Volume | | | | | |

These data clearly indicate that the tumor volumes decreased by 70% in all five tumors after the treatment with a p<0.01 statistical significance.

This experiment was enlightening as to reveal factors for direct injection of hydrogen peroxide and sodium hypochlorite made into a tumor mass. For example, the rate of injection for reactants should be such that the reactants cause blanching of the tumor as a result of infiltrating effects. Reactants can be injected either simultaneously or nonsimultaneously. With regard to the order of injection, it may be desirable to infiltrate the target site of injection first with sodium hypochlorite followed by injection with hydrogen peroxide because catalase naturally occurring in both the skin and blood cells will decompose hydrogen peroxide before it can interact with sodium hypochlorite if it is injected first.

The speed of injection should be determined by the blanching of the target site. This normally requires a rapid rate of injection, and is facilitated by the use of a 1 ml syringe, which can generate more hydrodynamic pressure than does larger syringes. The depth of injection is primarily determined by the target site being injected. For example, whereas a control site can be injected directly into the dermis of the skin, a cancerous lesion may have to be injected not only into the epidermis and dermis but also into the hypodermis, since it is desirable to have the reactants reach all the cancerous cells. Excess amounts of the reactants injected into surrounding normal tissue can be converted into sodium chloride, oxygen, and water by body's enzymatic systems. With regard to the concentration of reactants, it is desirable that reactants should be combined in a molecule for molecule amount to have maximum yield of singlet oxygen.

### Example 14

### Deuterated Solution I

Deuterated solutions are prepared from 3% deuterium peroxide in water and 6% sodium hypochlorite in water. The deuterium peroxide solution is stable and is stored in an ordinary container. The deuterium peroxide solution and the sodium hypochlorite solution are kept in separate containers until they are needed.

### Example 15

### Deuterated Solution II

Deuterated solutions are prepared from 3% hydrogen peroxide in deuterium oxide and 6% sodium hypochlorite in deuterium oxide. The concentration of deuterium oxide can be modified if necessary. Deuterium oxide can be obtained from the conventional source. The deuterium oxide solution of hydrogen peroxide and the deuterium oxide solution of sodium hypochlorite are stable and stored in ordinary containers. They are kept in separate containers until they are needed.

### Example 16

### Application of Deuterated Solution

The deuterated solutions of Example 14 or 15 are used in keratosis treatment of Examples 1 and 2, in sclerotic plaque treatment of Examples 3, 4, and 5, in equine carcinoma treatment of Examples 6 and 7, in decontamination of Example 8, in routine disinfection of Example 9, in topical antiseptic of Example 10, in wart treatment of Example 11, and in tumor treatment of Examples 12 and 13 without any substantial modifications. The same or enhanced singlet oxygen production is obtained by the deuterated solutions of Example 14 or 15.

### Example 17

### Radioactive Solution I and Its Application

In order to monitor a liver having tumor during or after the singlet oxygen treatment, ^{99m}Tc (half-life: 6 hours, and particle energy: gamma emission at 140 keV) is selected to be administered with singlet oxygen-forming reactants. The typical dose of ^{99m}Tc is a few mCi depending on body weight and age. 100 MBq of ^{99m}Tc-colloid or 150 MBq of ^{99m}mTc-HIDA (examples of ^{99m}Tc-complex used for liver diagnosis) is prepared and added to the solution of singlet oxygen-forming reactants. This ^{99m}Tc-labeled reactant solution is delivered to the liver containing tumor and the change of tumor during or after the singlet oxygen treatment is detected by the conventional method.

### Example 18

### Radioactive Solution II and Its Application

In order to treat a thyroid gland having thyreotoxicos (Graves disease with enlargement of the thyroid gland), ¹³¹I, having a half-life of 8.05 days and emitting a high-energy gamma ray of 364 keV, is chosen to be added to the singlet oxygen-producing reactants. The reactant solutions containing 200-1,000 MBq of ¹³¹I are administered to the thyroid gland having the disorder. Synergistic effect from singlet oxygen and therapeutic radioactive isotope is achieved in the treatment of the thyroid gland.

The entire contents of all documents cited in this specification is a part of the present disclosure, and all documents cited herein are hereby incorporated by reference.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

1-41. (Canceled).

42. An apparatus for singlet oxygen delivery comprising:
a) a first reservoir for containing at least one peroxide source;
b) a second reservoir for containing at least one hypochlorite anion source;
c) a first conduit connecting the first reservoir to at least one first delivery port; and
d) a second conduit connecting the second reservoir to at least one second delivery port;
wherein the at least one first delivery port and the at least one second delivery port allow a bodily fluid flow to carry the at least one peroxide source and the at least one hypochlorite anion source to the target site.

43. The apparatus according to claim 42, wherein the at least one first delivery port and the at least one second delivery port are a plurality of openings.

44. The apparatus according to claim 43, wherein the plurality of openings are located on the side of the apparatus.

45. The apparatus according to claim 42, further comprising at least one guide.

46. A kit for treating a tumor or precancerous lesion, said kit consisting of:
at least one peroxide source and at least one hypochlorite anion source in separate containers.

47. An apparatus for delivering singlet oxygen to a tumor or precancerous lesion, the apparatus comprising:
at least first and second syringe barrels for containing and delivering, respectively, at least one peroxide source and at least one hypochlorite anion source;
at least one mixing chamber for mixing the at least one peroxide source with the at least one hypochlorite source; and
at least one conduit for carrying the at least one peroxide source and the at least one hypochlorite source.

48. The apparatus according to claim 47, wherein the at least one conduit is located distal to the mixing chamber, with respect to the syringe barrels.

49. The apparatus according to claim 47, wherein the at least one conduit is located proximal to the mixing chamber, with respect to the syringe barrels.

47. A kit for treating a tumor or precancerous lesion, said kit comprising the apparatus according to claim 42 and at least one peroxide source and at least one hypochlorite anion source in separate containers.
